(19) United States
(12) Patent Application Publication
(10) Pub. No.: US 2017/0234867 A1
(43) Pub. Date: Aug. 17, 2017

(54) QUANTITATIVE LATERAL FLOW ASSAY

(71) Applicant: Astute Medical, Inc., San Diego, CA (US)

(72) Inventors: Joseph ANDERBERG, Encinitas, CA (US); Paul Harold MCPHERSON, Encinitas, CA (US); Ravi VIJAYENDRAN, Carlsbad, CA (US)

(73) Assignee: Astute Medical, Inc., San Diego, CA (US)

(21) Appl. No.: 15/444,178

(22) Filed: Feb. 27, 2017

Related U.S. Application Data
(62) Division of application No. 14/439,528, filed on Apr. 29, 2015, now abandoned.
(60) Provisional application No. 61/720,971, filed on Oct. 31, 2012.

(51) Int. Cl. G01N 33/558; G01N 33/68; G01N 33/543

(52) U.S. Cl. CPC G01N 33/558; G01N 33/54306; G01N 33/6893; G01N 33/54393; G01N 33/6845; G01N 33/54386; G01N 2333/4745; G01N 2333/8146

(57) ABSTRACT

The present invention relates to devices, kits, instruments and methods for quantitatively detecting multiple analytes in a sample. More specifically, the present invention relates to devices, kits, instruments and methods for quantitatively detecting multiple analytes with desired or targeted precision, and the uses thereof.

### CROSS-REFERENCE TO RELATED APPLICATIONS

This application claims the priority benefit of U.S. provisional application Ser. No. 61/720,971, filed Oct. 31, 2012, the content of which is incorporated by reference in its entirety.

### FIELD OF THE INVENTION

The present invention relates to devices, kits, instruments and methods for quantitatively detecting multiple analytes in a sample. More specifically, the present invention relates to devices, kits, instruments and methods for quantitatively detecting multiple analytes with desired or targeted precision, and the uses thereof.

### BACKGROUND OF THE INVENTION

Lateral flow immunoassays are widely used in many different areas of analytical chemistry and medicine.

Previous lateral flow immunoassay work is exemplified by U.S. patents and patent application publications: U.S. Pat. Nos. 5,602,040; 5,622,871; 5,656,503; 6,187,598; 6,228,660; 6,818,455; 2001/0008774; 2005/0244986; U.S. Pat. No. 6,352,862; 2003/0207465; 2003/0143755; 2003/0219908; U.S. Pat. Nos. 5,714,389; 5,989,921; 6,485,982; Ser. No. 11/035,047; U.S. Pat. Nos. 5,656,448; 5,559,041; 5,252,496; 5,728,587; 6,027,943; 6,506,612; 6,541,277; 6,737,277 B1; 5,073,484; 5,654,162; 6,020,147; 4,956,302; 5,120,643; 6,534,320; 4,942,522; 4,703,017; 4,743,560; 5,591,645; and RE 38,430 E.

There is a need for improved analytical technology to provide for multiplex lateral flow assays with improved assay precision. The present invention addresses this and other related needs.

### BRIEF SUMMARY OF THE INVENTION

In one aspect, the present invention provides a lateral flow test device for quantitatively detecting multiple analytes in a sample, which device comprises a porous matrix that comprises at least two distinct test locations on said porous matrix, each of said test locations comprising a test reagent that binds to an analyte or another binding reagent that binds to said analyte, or is an analyte or an analyte analog that competes with an analyte in said sample for binding to a binding reagent for said analyte, and said test reagents at said at least two test locations bind to at least two different analytes or different binding reagents that bind to said different analytes, or are different analytes or analyte analogs, wherein a liquid sample flows laterally along said test device and passes said test locations to form a detectable signal to determine amounts of said multiple analytes in said sample.

In another aspect, the present invention provides a method for quantitatively detecting multiple analytes in a sample, which method comprises: a) contacting a liquid sample with the above test device, wherein the liquid sample is applied to a site of the test device upstream of the test locations; b) transporting multiple analytes, if present in the liquid sample, and a labeled reagent to the test locations; and c) assessing a detectable signal at the test locations to determine the amounts of the multiple analytes in the sample.

In still another aspect, the present invention provides a system for quantitatively detecting multiple analytes in a sample, which system comprises: a) the above test device; and b) a reader that comprises a light source and a photodetector to detect a detectable signal.

In yet another aspect, the present invention provides a kit for quantitatively detecting multiple analytes in a sample, which kit comprises: a) the above test device; and b) an instruction for using the test device to quantitatively detect multiple analytes in a sample.

The principles of the present test devices, kits, systems and methods can be applied, or can be adapted to apply, to the lateral flow test devices and assays known in the art. For example, the principles of the present test devices, kits, systems and methods can be applied, or can be adapted to apply, to the lateral flow test devices and assays disclosed and/or claimed in the following patents and applications: U.S. Pat. Nos. 5,073,484, 5,654,162, 6,020,147, 4,695,554, 4,703,017, 4,743,560, 5,591,645, RE 38,430 E, 5,602,040, 5,633,871, 5,656,503, 6,187,598, 6,228,660, 6,818,455, 7,109,042, 6,352,862, 7,238,537, 7,384,796, 7,407,813, 5,714,389, 5,989,921, 6,485,982, 5,120,643, 5,578,577, 6,534,320, 4,956,302, RE 39,664 E, 5,252,496, 5,559,041, 5,728,587, 6,027,943, 6,506,612, 6,541,277, 6,737,277, 7,175,992 B2, 7,691,595 B2, 6,770,487 B2, 7,247,500 B2, 7,662,643 B2, 5,712,170, 5,965,458, 7,371,582 B2, 7,476,549 B2, 7,633,620 B2, 7,815,853 B2, 6,267,722 B1, 6,394,952 B1, 6,867,051 B1, 6,936,476 B1, 7,270,970 B2, 7,239,394 B2, 7,315,378 B2, 7,317,532 B2, 7,616,315 B2, 7,521,259 B2, 7,521,260 B2, US 2005/0221504 A1, US 2005/0221505 A1, US 2006/0240541 A1, US 2007/0143035 A1, US 2007/0185679 A1, US 2008/0028261 A1, US 2009/0180925 A1, US 2009/0180926 A1, US 2009/0180927 A1, US 2009/0180928 A1, US 2009/0180929 A1, US 2009/0214383 A1, US 2009/0269858A1, U.S. Pat. No. 6,777,198, US 2009/0311724 A1, US 2009/0117006 A1, U.S. Pat. Nos. 7,256,053, 6,916,666, 6,812,038, 5,710,005, 6,140,134, US 2010/0143941 A1, U.S. Pat. Nos. 6,140,048, 6,756,202, 7,205,553, 7,679,745, US 2010/0165338 A1, US 2010/0015611 A1, U.S. Pat. Nos. 5,422,726, 5,596,414, 7,178,416, 7,784,678 B2, US 2010/094564 A1, US 2010/0173423 A1, US 2009/0157023 A1, U.S. Pat. Nos. 7,785,899, 7,763,454 B2, US 2010/0239460 A1, US 2010/0240149 A1, U.S. Pat. Nos. 7,796,266 B2, 7,815,854 B2, US 2005/0244953 A1, US 2007/0121113 A1, US 2003/0119202 A1, US 2010/0311181 A1, U.S. Pat. No. 6,707,554 B1, 6,194,222 B1, 7,713,703, EP 0,149,168 A1, EP 0,323,605 A1, EP 0,250,137 A2, GB 1,526,708 and WO99/40438.

### BRIEF DESCRIPTION OF THE DRAWINGS

FIG. 1 illustrates an exemplary lateral flow device.

FIG. 2 provides the top view and the side view of the exemplary lateral flow device illustrated in FIG. 1.

FIG. 3 illustrates an exemplary test cartridge, e.g., NephroCheck Test cartridge.

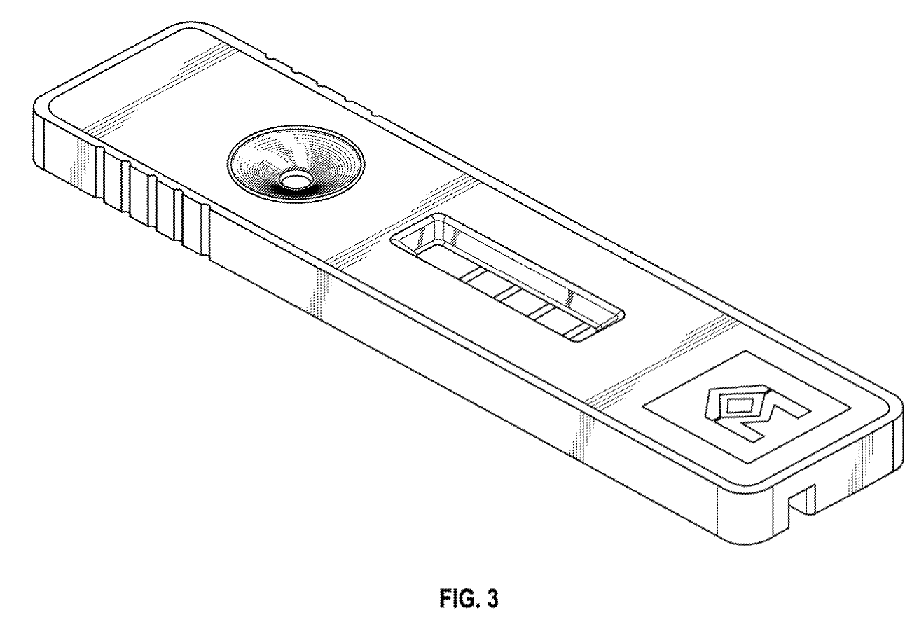

FIG. 4 illustrates an exemplary meter or reader for quantitatively detecting signals from a lateral flow device, e.g., Astute 140 Meter.

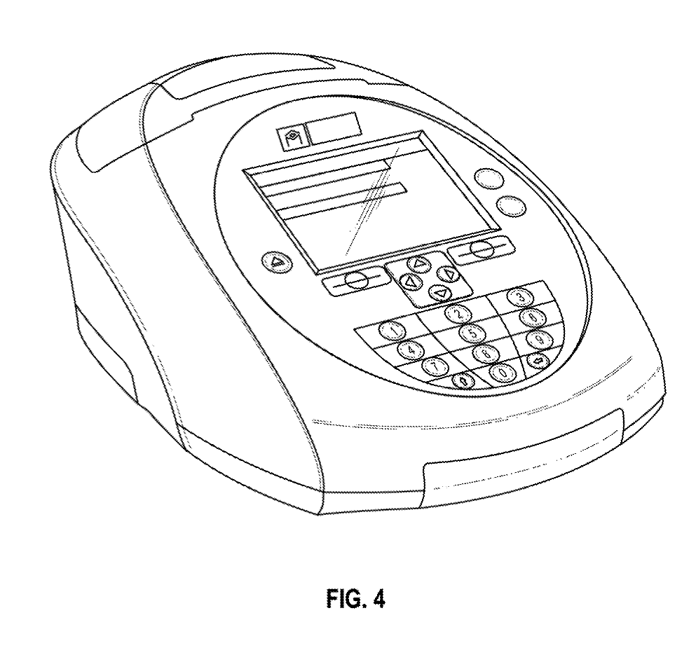

FIG. 5 illustrates an exemplary test cartridge, e.g., NephroCheck Test cartridge.

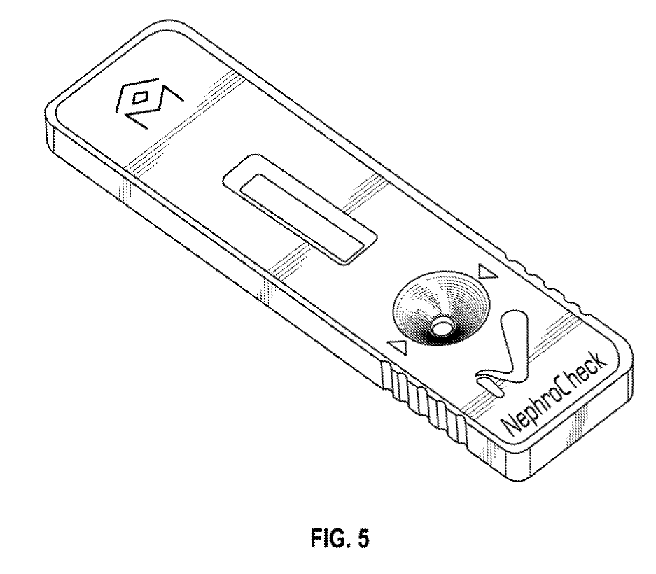

FIG. 6 illustrates an exemplary NEPHROCHECK™ Test Preparation Process.

FIG. 7 illustrates relative risk for moderate or severe AKI by tertiles of NEPHROCHECK Test values. *p<0.001 for risk relative to the first tertile, **p<0.001 for risk relative to the first and second tertiles.

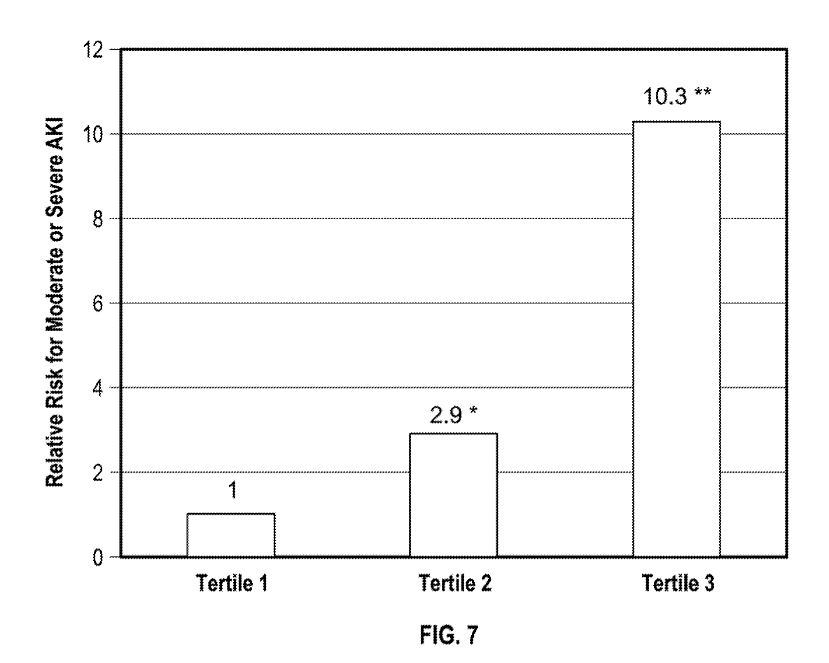

### DETAILED DESCRIPTION OF THE INVENTION

### A. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “determine amounts of said multiple analytes in said sample” means that each of the analytes is determined with a precision, or coefficient of variation (CV), at about 30% or less, at analyte level(s) or concentration(s) that encompasses one or more desired threshold values of the analyte(s), and/or at analyte level(s) or concentration(s) that is below, at about low end, within, at about high end, and/or above one or more desired reference ranges of the analyte(s). In some embodiments, it is often desirable or important to have higher precision, e.g., CV less than 30%, 25%, 20%, 15%, 10%, 9%, 8%, 7%, 6%, 5%, 4%, 3%, 2%, 1%, 0.5%, 0.1%, or smaller. In other embodiments, it is often desirable or important that the analytes are quantified with a desired or required CV at analyte level(s) or concentration(s) that is substantially lower than, at about, or at, and/or substantially higher than the desired or required threshold values of the analyte(s). In still other embodiments, it is often desirable or important that the analytes are quantified with a desired or required CV at analyte level(s) or concentration(s) that is substantially lower than the low end of the reference range(s), that encompasses at least 10%, 20%, 30%, 40%, 50%, 60%, 70%, 80%, 90%, 95%, or the entire reference range(s), and/or that is substantially higher than the high end of the reference range(s).

As used herein, an analyte level or concentration “at about” a threshold value or a particular point, e.g., low or high end, of a reference range, means that the analyte level or concentration is at least within plus or minus 20% of the threshold value or the particular point, e.g., low or high end, of the reference range. In other words, an analyte level or concentration “at about” a threshold value or a particular point of a reference range means that the analyte level or concentration is at from 80% to 120% of the threshold value or a particular point of the reference range. In some embodiments, an analyte level or concentration “at about” a threshold value or a particular point of a reference range means that the analyte level or concentration is at least within plus or minus 15%, 10%, 5%, 4%, 3%, 2%, 1%, or equals to the threshold value or the particular point of the reference range.

As used herein, analyte level or concentration that is “substantially lower than” a threshold value or the low end of a reference range means that the analyte level or concentration is at least within minus 50% of the threshold value or the low end of the reference range. In other words, an analyte level or concentration that is “substantially lower than” the threshold value or the low end of the reference range means that the analyte level or concentration is at least at 50% of the threshold value or the low end of the reference range. In some embodiments, analyte level or concentration that is “substantially lower than” the threshold value or the low end of the reference range means that the analyte level or concentration is at least at 60%, 70%, 80%, 90%, 95%, 96%, 97%, 98%, 99% of the threshold value or the low end of the reference range.

As used herein, analyte level or concentration that is “substantially higher than” a threshold value or the high end of a reference range means that the analyte level or concentration is at least within plus 5 folds of the threshold value or the high end of the reference range. In other words, an analyte level or concentration that is “substantially higher than” the threshold value or the high end of the reference range means that the analyte level or concentration is at 101% to 5 folds of the threshold value or the high end of the reference range. In some embodiments, analyte level or concentration that is “substantially higher than” the threshold value or the high end of the reference range means that the analyte level or concentration is at least at 101%, 102%, 103%, 104%, 105%, 110%, 120%, 130%, 140%, 150%, 2 folds, 3 folds, 4 folds or 5 folds of the threshold value or the high end of the reference range.

As used herein, “threshold value” refers to an analyte level or concentration obtained from samples of desired subjects or population, e.g., values of analyte level or concentration found in normal, clinically healthy individuals, analyte level or concentration found in “diseased” subjects or population, or analyte level or concentration determined previously from samples of desired subjects or population. If a “normal value” is used as a “threshold range,” depending on the particular test, a result can be considered abnormal if the value of the analyte level or concentration is more or less than the normal value. A “threshold value” can be based on calibrated or un calibrated analyte levels or concentrations.

As used herein, “reference range” refers to a range of analyte level or concentration obtained from samples of a desired subjects or population, e.g., the range of values of analyte level or concentration found in normal, clinically healthy individuals, the range of values of analyte level or concentration found in “diseased” subjects or population, or the range of values of analyte level or concentration determined previously from samples of desired subjects or population. If a “normal range” is used as a “reference range,” a result is considered abnormal if the value of the analyte level or concentration is less than the lower limit of the normal range or is greater than the upper limit. A “reference range” can be based on calibrated or un calibrated analyte levels or concentrations.

As used herein, “antibody” refers a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.” An “antibody” may be naturally occurring or man-made such as monoclonal antibodies produced by conventional hybridoma technology, various display methods, e.g., phage display, and/or a functional fragment thereof.

The term “epitope” refers to an antigenic determinant capable of specific binding to an antibody. Epitopes usually or often consist of chemically active surface groupings of molecules such as amino acids or sugar side chains and can have specific three dimensional structural characteristics, as well as specific charge characteristics. Conformational and nonconformational epitopes are distinguished in that the binding to the former but not the latter is lost in the presence of denaturing solvents.

As used herein, “monoclonal antibody” refers to an antibody obtained from a population of substantially homogeneous antibodies, i.e., the antibodies comprising the population are identical except for possible naturally occurring mutations that are present in minor amounts. As used herein, a “monoclonal antibody” further refers to functional fragments of monoclonal antibodies.

As used herein, “mammal” refers to any of the mammalian class of species, preferably human (including humans, human subjects, or human patients). Mammals include, but are not limited to, farm animals, sport animals, pets, primates, horses, dogs, cats, mice and rats.

As used herein, “treatment” means any manner in which a condition, disorder or disease or the symptom(s) of a condition, disorder or disease is ameliorated or otherwise beneficially altered. Treatment also encompasses any pharmaceutical use of the compositions herein.

As used herein, “disease or disorder” refers to a pathological condition in an organism resulting from, e.g., infection or genetic defect, and characterized by identifiable symptoms or by laboratory tests or other diagnostic and assessment criteria known to one skilled in the art.

As used herein, the term “subject” is not limited to a specific species or sample type. For example, the term “subject” may refer to a patient, and frequently a human patient. However, this term is not limited to humans and thus encompasses a variety of mammalian or other species.

As used herein, “afflicted” as it relates to a disease or disorder refers to a subject having or directly affected by the designated disease or disorder.

As used herein, the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, a “binding reagent” refers to any substance that binds to a target or an analyte with desired affinity and/or specificity. Non-limiting examples of the binding reagent include cells, cellular organelles, viruses, particles, microparticles, molecules, or an aggregate or complex thereof, or an aggregate or complex of molecules. Exemplary binding reagents can be an amino acid, a peptide, a protein, e.g., an antibody or receptor, a nucleoside, a nucleotide, an oligonucleotide, a nucleic acid, e.g., DNA or RNA, a vitamin, a monosaccharide, an oligosaccharide, a carbohydrate, a lipid, an aptamer and a complex thereof.

As used herein, the term “specifically binds” refers to the specificity of a binding reagent, e.g., an antibody or an aptamer, such that the binding reagent preferentially binds to a defined target or analyte. An binding reagent “specifically binds” to a target if it binds with greater affinity, avidity, more readily, and/or with greater duration than it binds to other substances. For example, a binding reagent that specifically binds to a target may bind to the target analyte with at least about 10%, at least about 20%, at least about 30%, at least about 40%, at least about 50%, at least about 60%, at least about 70%, at least about 80%, at least about 90% or more, greater affinity as compared to binding to other substances; or with at least about two-fold, at least about five-fold, at least about ten-fold or more of the affinity for binding to a target analyte as compared to its binding to other substances. Recognition by a binding reagent of a target analyte in the presence of other potential interfering substances is also one characteristic of specifically binding. Preferably, a binding reagent, e.g., an antibody or an aptamer, that is specific for or binds specifically to a target analyte, avoids binding to a significant percentage of non-target substances, e.g., non-target substances present in a testing sample. In some embodiments, a binding reagent avoids binding greater than about 90% of non-target substances, although higher percentages are clearly contemplated and preferred. For example, a binding reagent can avoid binding about 91%, about 92%, about 93%, about 94%, about 95%, about 96%, about 97%, about 98%, about 99%, about 99% and about 99.9% or more of non-target substances. In other embodiments, a binding reagent can avoid binding greater than about 10%, 20%, 30%, 40%, 50%, 60%, or 70%, or greater than about 75%, or greater than about 80%, or greater than about 85% of non-target substances.

As used herein, “stringency” of nucleic acid hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Current Protocols in Molecular Biology (Ausubel et al. eds., Wiley Interscience Publishers, 1995); Molecular Cloning: A Laboratory Manual (J. Sambrook, E. Fritsch, T. Maniatis eds., Cold Spring Harbor Laboratory Press, 2d ed. 1989); Wood et al., Proc. Natl. Acad. Sci. USA, 82:1585-1588 (1985).

As used herein the term “isolated” refers to material removed from its original environment, and is altered from its natural state. For example, an isolated polypeptide could be coupled to a carrier, and still be “isolated” because that polypeptide is not in its original environment.

### B. Devices and Kits for Quantitatively Detecting Multiple Analytes in a Sample

In one aspect, the present invention provides a lateral flow test device for quantitatively detecting multiple analytes in a sample, which device comprises a porous matrix that comprises at least two distinct test locations on said porous matrix, each of said test locations comprising a test reagent that binds to an analyte or another binding reagent that binds to said analyte, or is an analyte or an analyte analog that competes with an analyte in said sample for binding to a binding reagent for said analyte, and said test reagents at said at least two test locations bind to at least two different analytes or different binding reagents that bind to said different analytes, or are different analytes or analyte analogs, wherein a liquid sample flows laterally along said test device and passes said test locations to form a detectable signal to determine amounts of said multiple analytes in said sample.

The present assays can be used to determine amounts of multiple analytes with desired precision. Typically, the amount of each of the multiple analytes is determined with a precision, or coefficient of variation (CV), at about 30% or less, at analyte level(s) or concentration(s) that encompasses one or more desired threshold values of the analyte(s), and/or at analyte level(s) or concentration(s) that is below, at about low end, within, at about high end, and/or above one or more desired reference ranges of the analyte(s).

In some embodiments, it is often desirable or important to have higher precision, e.g., CV less than 30%, 25%, 20%, 15%, 10%, 9%, 8%, 7%, 6%, 5%, 4%, 3%, 2%, 1%, 0.5%, 0.1%, or smaller, at the desired analyte level(s) or concentration(s).

In other embodiments, it is often desirable or important that the analytes are quantified with a desired or required CV at analyte level(s) or concentration(s) that is substantially lower than, at about, or at, and/or substantially higher than the desired or required threshold values of the analyte(s). The precision or CV standard can be applied to the assays wherein the amount of each analyte is determined and compared to its corresponding threshold value individually. For example, each of the analytes can be quantified with a desired or required CV at analyte level or concentration that is substantially lower than the desired or required threshold values of the analyte. In another example, each of the analytes can be quantified with a desired or required CV at analyte level or concentration that is at about, or at, the desired or required threshold value of the analyte. In still another example, each of the analytes can be quantified with a desired or required CV at analyte level or concentration that is substantially higher than the desired or required threshold values of the analyte. In yet another example, each of the analytes can be quantified with a desired or required CV at analyte level or concentration range that is from substantially lower than to substantially higher than the desired or required threshold values of the analyte. The multiple analytes can be quantified with the same level or different levels of CV, or with the same range or different ranges of CV. The precision or CV standard can also be applied to the assays wherein the amounts of the multiple analytes are quantified and converted into a composite amount and the composite analyte amount is compared to its corresponding composite threshold value.

In still other embodiments, it is often desirable or important that the analytes are quantified with a desired or required CV at analyte level(s) or concentration(s) that is substantially lower than the low end of the reference range(s), that encompasses a portion or the entire reference range(s), and/or that is substantially higher than the high end of the reference range(s). The precision or CV standard can be applied to the assays wherein the amount of each analyte is determined and compared to its corresponding reference range individually. For example, each of the analytes can be quantified with a desired or required CV at analyte level or concentration that is substantially lower than the low end of the reference range of the analyte. In another example, each of the analytes can be quantified with a desired or required CV at analyte level or concentration that encompasses 10%, 20%, 30%, 40%, 50%, 60%, 70%, 80%, 80%, 95%, or the entire reference range of the analyte. In still another example, each of the analytes can be quantified with a desired or required CV at analyte level or concentration that is substantially higher than the high end of the reference range of the analyte. In yet another example, each of the analytes can be quantified with a desired or required CV at analyte level or concentration range that is from substantially lower than the low end of the reference range to substantially higher than the high end of the reference range of the analyte. The multiple analytes can be quantified with the same level or different levels of CV, or with the same range or different ranges of CV. The precision or CV standard can also be applied to the assays wherein the amounts of the multiple analytes are quantified and converted into a composite amount and the composite analyte amount is compared to its corresponding composite reference range.

Precision can be assessed by any suitable methods. Precision is generally expressed in relative terms as the coefficient of variation (CV). The coefficient of variation is typically determined by C.V.=100×S.D./mean.

A variety of methods may be used by the skilled artisan to arrive at a desired threshold value for use in the present assays. For example, the threshold value may be determined from a population of normal subjects by selecting a concentration representing the 1^{st}, 5^{th}, 10^{th}, 15^{th}, 25^{th}, 50^{th}, 75th, 85th, 90th, 95th, or 99th percentile of a marker, e.g., a kidney injury marker, measured in such normal subjects. Alternatively, the threshold value may be determined from a “diseased” population of subjects, e.g., those suffering from an injury or having a predisposition for an injury (e.g., progression to acute kidney injury or acute renal failure (ARF) or some other clinical outcome such as death, dialysis, renal transplantation, etc.), by selecting a concentration representing the 1^{st}, 5^{th}, 10^{th}, 15^{th}, 25^{th}, 50^{th}, 75th, 85th, 90th, 95th, or 99th percentile of a marker measured in such subjects. In another alternative, the threshold value may be determined from a prior measurement of a marker in the same subject; that is, a temporal change in the level of a marker in the subject may be used to assign risk to the subject. In still another alternative, the threshold value may be a value that is commonly recognized for a disease, disorder or a condition.

The foregoing discussion is not meant to imply, however, that the markers, e.g., kidney injury markers, of the present invention must be compared to corresponding individual thresholds. Methods for combining assay results can comprise the use of multivariate logistical regression, loglinear modeling, neural network analysis, n-of-m analysis, decision tree analysis, calculating ratios or products of markers, etc. This list is not meant to be limiting. In these assays, a composite result which is determined by combining individual markers may be treated as if it is itself a marker; that is, a threshold may be determined for the composite result as described herein for individual markers, and the composite result for an individual patient compared to this threshold. The individual analyze amounts can be combined in any suitable way to produce a composite amount, e.g., a composite amount being a sum, subtraction, multiplication, ratio, product, or proportion of, between or among the individual analyte amounts.

Test results can also be interpreted with respect to a reference range, e.g., the range of values found in normal, clinically healthy individuals. A result is considered outside the reference range if the test result is less than the lower limit of the reference range or is greater than the upper limit of the reference range. A reference range is often determined from measurements on samples from a large number, e.g., several hundred, of the individuals of the intended or desired population. In some embodiments, when results are plotted in histogram fashion, a distribution such as that illustrated in Norman, G. R. and Streiner, D. L., Biostatistics: The Bare Essentials, Shelton, Conn.: People's Medical Publishing House, 2008. In this example, a reference range can be determined by lower and upper limit values, as represented by test result values A and B in Norman, G. R. and Streiner, D. L., Biostatistics: The Bare Essentials, Shelton, Conn.: People's Medical Publishing House, 2008, which include an intended or desired percentage of all of the values, e.g., 1%, 5%, 10% 25%, 50%, 70%, 75%, 80%, 85%, 90%, or 95% of all of the values. The distribution of values, in many cases, may be Gaussian, bell-shaped, or uniform, as in shown in Norman, G. R. and Streiner, D. L., Biostatistics: The Bare Essentials, Shelton, Conn.: People's Medical Publishing House, 2008. A reference range can be determined by any suitable methods, standard or formula. For example, a reference range can be determined from the mean value and the standard deviation (S.D.), e.g.:

lower limit (A)=mean value−2 S.D.

upper limit (B)=mean value+2 S.D.

Not all test results from the intended or desired population, e.g., a clinically normal population, distribute uniformally. Sometimes, a more tedious, nonparametric procedure can be used to determine the lower and upper limits which include an intended or desired percentage of all of the values, e.g., 70%, 75%, 80%, 85%, 90%, or 95% of all of the values of the population.

In some cases the upper and lower limits comprising an intended or desired percentage of all of the values, e.g., 70%, 75%, 80%, 85%, 90%, or 95% of a normal population may not the appropriate reference range. For example, total serum cholesterol is a case in which the usually quoted reference range is determined as a “healthy” range on the basis of results from long term epidemiologic studies, such as the Framingham study. In other cases, of which serum creatinine is an example, it is appropriate to compare a current value to a previously determined value.

The ability of a particular test or combination of tests to distinguish two populations can be established using receiver operating characteristic (ROC) analysis. (See e.g., Metz, Semin. Nucl. Med., 8(4):283-98 (1978)). For example, ROC curves established from a “first” subpopulation which is predisposed to one or more future changes in a diseased status, e.g., renal status, and a “second” subpopulation which is not so predisposed can be used to calculate a ROC curve, and the area under the curve provides a measure of the quality of the test. Preferably, the tests described herein provide a ROC curve area greater than 0.5, preferably at least 0.6, more preferably 0.7, still more preferably at least 0.8, even more preferably at least 0.9, and most preferably at least 0.95.

In certain aspects, the measured concentration of one or more analytes or biomarkers, e.g., kidney injury markers, or a composite of such markers, may be treated as continuous variables. For example, any particular concentration can be converted into a corresponding probability of a future reduction in renal function for the subject, the occurrence of an injury, a classification, etc. In yet another alternative, a threshold that can provide an acceptable level of specificity and sensitivity in separating a population of subjects into “bins” such as a “first” subpopulation (e.g., which is predisposed to one or more future changes in disease or renal status, the occurrence of an injury, a classification, etc.) and a “second” subpopulation which is not so predisposed. A threshold value can be selected to separate this first and second population by one or more of the following measures of test accuracy:

an odds ratio greater than 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less;

a specificity of greater than 0.1, 0.2, 0.3, 0.4 or 0.5, preferably at least about 0.6, more preferably at least about 0.7, still more preferably at least about 0.8, even more preferably at least about 0.9 and most preferably at least about 0.95, with a corresponding sensitivity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;

at least about 75% sensitivity, combined with at least about 75% specificity;

a positive likelihood ratio (calculated as sensitivity/(1-specificity)) of greater than 1, at least about 2, more preferably at least about 3, still more preferably at least about 5, and most preferably at least about 10; or

a negative likelihood ratio (calculated as (1-sensitivity)/specificity) of less than 1, less than or equal to about 0.5, more preferably less than or equal to about 0.3, and most preferably less than or equal to about 0.1.

In some embodiments, the term “about” in the context of any of the above measurements may refer to +/−5% of a given measurement.

Multiple thresholds may also be used to assess a disease status, e.g., renal status, in a subject. For example, a “first” subpopulation which is predisposed to one or more future changes in renal status, the occurrence of an injury, a classification, etc., and a “second” subpopulation which is not so predisposed can be combined into a single group. This group can then be subdivided into three or more equal parts (known as tertiles, quartiles, quintiles, etc., depending on the number of subdivisions). An odds ratio is assigned to subjects based on which subdivision they fall into. If one considers a tertile, the lowest or highest tertile can be used as a reference for comparison of the other subdivisions. This reference subdivision is assigned an odds ratio of 1. The second tertile is assigned an odds ratio that is relative to that first tertile. That is, someone in the second tertile might be 3 times more likely to suffer one or more future changes in renal status in comparison to someone in the first tertile. The third tertile is also assigned an odds ratio that is relative to that first tertile.

The matrix can comprise any suitable material(s). For example, the matrix can comprise nitrocellulose, glass fiber, polypropylene, polyethylene (preferably of very high molecular weight), polyvinylidene flouride, ethylene vinylacetate, acrylonitrile and/or polytetrafluoro-ethylene.

Depending on the intended test format and goals, the test reagents can be any suitable substances. In some embodiments, the test reagents bind to at least two different analytes. Preferably, the test reagents specifically bind to at least two different analytes. In other embodiments, the test reagents are different analytes or analyte analogs. In some embodiments, the test reagents are inorganic molecules, organic molecules or a complex thereof. Exemplary organic molecules include an amino acid, a peptide, a protein, a nucleoside, a nucleotide, an oligonucleotide, a nucleic acid, a vitamin, a monosaccharide, an oligosaccharide, a carbohydrate, a lipid and a complex thereof. In other embodiments, the test reagents can be an antigen, an antibody or an aptamer.

The matrix can have any suitable form. In some embodiments, the matrix can be in the form a strip or a circle. In other embodiments, the matrix can be a single element or can comprise multiple elements.

The test device can comprise additional elements. In some embodiments, the test device can further comprise a sample application element upstream from and in fluid communication with the matrix. In other embodiments, the test device can further comprise a liquid absorption element downstream from and in fluid communication with the matrix.

In some embodiments, at least a portion of the matrix is supported by a solid backing. In other embodiments, half, more than half or all portion of the matrix is supported by a solid backing. The solid backing can be made of any suitable material, e.g., solid plastics. If the test device comprises electrode or other electrical elements, the solid backing should generally comprise non-conductive materials.

The test device can further comprise a dried, labeled reagent. In some embodiments, a portion of the matrix, upstream from the test locations, can comprise a dried, labeled reagent, the labeled reagent being capable of being moved by a liquid sample and/or a further liquid, e.g., a sample transporting fluid or a washing fluid, to the test locations and/or a control location, e.g., a positive and/or negative control location, to generate a detectable signal.

The test device can comprise any suitable number or type of dried, labeled reagent. In some embodiments, the test device comprises one labeled reagent for one analyte. In other embodiments, the test device comprises one labeled reagent for multiple analytes. In still other embodiments, the test device comprises multiple labeled reagents for one analyte.

The dried, labeled reagent can be located at any suitable locations. In some embodiments, the dried, labeled reagent is located downstream from a sample application place on the test device. In other embodiments, the dried, labeled reagent is located upstream from a sample application place on the test device. In still other embodiments, the test device further comprises, upstream from the test locations, a conjugate element that comprises a dried, labeled reagent, the labeled reagent being capable of moved by a liquid sample and/or a further liquid to the test locations and/or a control location, e.g., a positive and/or negative control location, to generate a detectable signal. The conjugate element can be located downstream from a sample application place on the test device. Alternatively, the conjugate element can be located upstream from a sample application place on the test device.

The labeled reagent can have any suitable binding affinity and/or specificity. In some embodiments, the labeled reagent binds, and preferably specifically binds, to one or more analytes in the sample. In other embodiments, the test device comprises multiple labeled reagents, wherein each of the labeled reagents competes with a different analyte in the sample for binding to a binding reagent for the analyte at a test location.

Any suitable label can be used depending on the intended detection methods. The label can be a direct label or an indirect label. A direct label can be detected by an instrument, device or naked eyes without further step to generate a detectable signal. A visual direct label, e.g., a gold or latex particle label, can be detected by naked eyes. An indirect label, e.g., an enzyme label, requires further step to generate a detectable signal. In some embodiments, the label is a soluble label, such as a colorimetric, radioactive, enzymatic, luminescent or fluorescent label. Exemplary fluorescent label includes the DyLight Fluor family of fluorescent dyes, e.g., DyLight 350, DyLight 405, DyLight 488, DyLight 550, DyLight 594, DyLight 633, DyLight 650, DyLight 680, DyLight 755 and DyLight 800 produced by Dyomics in collaboration with Thermo Fisher Scientific. In other embodiments, the label is a particle or particulate label, such as a particulate direct label, or a colored particle label. Exemplary particle or particulate labels include colloidal gold label, latex particle label, nanoparticle label and quantum dot label. Depending on the specific configurations, the labels such as colorimetric, radioactive, enzymatic, luminescent or fluorescent label, can be either a soluble label or a particle or particulate label.

The labeled reagent can be dried in the presence of a material that: a) stabilizes the labeled reagent; b) facilitates solubilization or resuspension of the labeled reagent in a liquid; and/or c) facilitates mobility of the labeled reagent. The exemplary material can be a protein, e.g., a casein or BSA, a peptide, a polysaccharide, a sugar, a polymer, e.g., polyvinylpyrrolidone (PVP-40), a gelatin, a detergent, e.g., Tween-20, and a polyol, e.g., mannitol. See e.g., U.S. Pat. Nos. 5,120,643 and 6,187,598. In some embodiments, the labeled reagent, e.g., a fluorescently labeled antibody, can be conjugated to polyethylene glycol (PEG) and/or polyethylene oxide (PEO). The presence of PEG and/or PEO can increase solubility, prolong stability and minimizes nonspecific binding of the labeled reagent. Although not to be bound by a particular theory, the presence of PEG and/or PEO can minimize nonspecific binding of the labeled reagent by causing the binding reagents or antibodies to sterically repel one another as well as other proteins and/or surfaces, e.g., surfaces of a container or the test device. PEG and/or PEO can be conjugated to the labeled reagent by any suitable ways. For example, PEG and/or PEO can be conjugated to the labeled reagent via various amines, e.g., primary amines, and/or sulfhydryl groups.

The test device can further comprise a control location for any suitable purpose. In some embodiments, a control location can comprise means for indicating proper flow of the liquid sample, means for indicating that the labeled reagent is added to the device and/or means for indicating that the labeled reagent is properly solubilized or dispersed, e.g., a labeled reagent added by an operator and/or a labeled reagent embedded on a test device. The means can comprise a substance that will generate a detectable signal, e.g., fluorescent, color or electrical signal, once a liquid flow along or through the control location. For example, a labeled binding partner, e.g., a labeled avidin or strepavidin, can be dried on the device. The labeled binding partner can be transported to a control location with an immobilized corresponding binding partner, e.g., biotin, to generate a detectable signal at the control location. The detection of the signal at the control location can be used to indicate proper addition and flow of sample or other liquid, and/or proper solubilization, suspension and transportation of the labeled reagents to the intended locations.

In other embodiments, a control location can comprise means for indicating a valid test result. In one example, the means comprises a binding reagent that binds to a binding reagent with a detectable label that also binds to the analyte. In another example, the means comprises a binding reagent that binds to a binding reagent with a detectable label that does not bind to the analyte. In still another example, the means comprises a binding reagent that binds to a substance in a test sample that is not a target analyte.

In still other embodiments, a control location can comprise means for indicating non-specific or unintended specific binding, or indicating heterophilic antibody interference, e.g., human anti-mouse antibody (HAMA) interference. In still other embodiments, a control location can comprise means for generating a control signal that is compared to signals at the test locations in determining amounts of the multiple analytes. The test device can comprise a single or multiple control locations, e.g., a positive control location and a negative control location.

The analytes and/or the labeled reagent can be transported to the test locations by any suitable methods. In some embodiments, a sample liquid alone is used to transport the analytes and/or the labeled reagent to the test locations. In other embodiments, a developing liquid is used to transport the analytes and/or the labeled reagent to the test locations. In still other embodiments, a combination of a sample liquid and a developing liquid is used to transport the analytes and/or the labeled reagent to the test locations.

The test device can further comprise a housing that covers at least a portion of the test device, wherein the housing comprises a sample application port to allow sample application upstream from or to the test locations and an optic opening around the test locations to allow signal detection at the test locations. The optic opening can be achieved in any suitable way. For example, the optic opening can simply be an open space. Alternatively, the optic opening can be a transparent cover.

In some embodiments, the housing covers the entire test device. In other embodiments, at least a portion of the sample receiving portion of the matrix or the sample application element is not covered by the housing and a sample or a buffer diluent is applied to the portion of the sample receiving portion of the matrix or the sample application element outside the housing and is then transported to the test locations. The housing can comprise any suitable material. For example, the housing can comprise a plastic material. In another example, the housing, whether in part or in its entirety, can comprise an opaque, translucent and/or transparent material.

The present test device can be used for quantitatively detecting any suitable number of analytes. For example, the present test device can be used for quantitatively detecting 2, 3, 4, 5, 6, 7, 8, 9, 10 or more analytes. The test device can be used for any suitable purpose. For example, the present test device can be used for quantitatively detecting multiple analytes that are diagnostic, prognostic, risk assessment, stratification and/or treatment monitoring markers.

The present test device can be used for quantitatively detecting any suitable analytes. Exemplary analytes include markers for diseases or conditions such as infectious diseases, parasitic diseases, neoplasms, diseases of the blood and blood-forming organs, disorders involving the immune mechanism, endocrine, nutritional and metabolic diseases, mental and behavioural disorders, diseases of the nervous system, diseases of the eye and adnexam, diseases of the ear and mastoid process, diseases of the circulatory system, diseases of the respiratory system, diseases of the digestive system, diseases of the skin and subcutaneous tissue, diseases of the musculoskeletal system and connective tissue, diseases of the genitourinary system, pregnancy, childbirth and the puerperium, conditions originating in the perinatal period, congenital malformations, deformations, chromosomal abnormalities, injury, poisoning, consequences of external causes, external causes of morbidity and mortality. (See e.g., International Statistical Classification of Diseases and Related Health Problems, World Health Organization). In some embodiments, the analytes are markers for acute coronary syndrome (ACS), abdominal pain, cerebrovascular injury, kidney injury, e.g., acute kidney injury or chronic kidney disease, or sepsis.

In other embodiments, the present device can be used for quantitatively detecting any suitable markers for kidney injury. Exemplary markers for kidney injury include insulin-like growth factor-binding protein 7 (or IGFBP7 or FSTL2 or IBP-7 or IGF-binding protein 7 or IGFBP-7 or IGFBP-7v or IGFBPRP1 or IGFBP-rP1 or MAC25 or MAC-25 or MAC 25 or PGI2-stimulating factor or AGM), metallopeptidase inhibitor 2 (or CSC-21K or metalloproteinase inhibitor 2 or TIMP-2 or tissue inhibitor of metalloproteinases 2 or TIMP2 or TIMP 2), neutrophil elastase (or bone marrow serine protease or ELA2 or elastase-2 or HLE or HNE or human leukocyte elastase or medullasin or neutrophil elastase or PMN-E or PMN elastase or SCN1 or ELANE or elastase neutrophil expressed or elastase 2 or neutrophil-derived elastase or granulocyte-derived elastase or polymorphonuclear elastase or leukocyte elastase), hyaluronic acid (or hyaluronan or hyaluronate), alpha-1 antitrypsin (A1AT, Alpha-1 protease inhibitor, alpha1AT, serine or cysteine proteinase inhibitor, AAT, PI, PI1, serine or cysteine proteinase inhibitor, clade A, member 1, alpha1AT, A1A, or serpin A1), serum amyloid p component (amyloid P component), β-2 glycoprotein, NGAL, KIM-1, Cystatin C, serum creatinine, L-FABP, IL-18, pi-GST, alph-GST, Clusterin. In still other embodiments, the present devices can be used for quantitatively detecting at least 2, 3, 4, 5, 6 or all 7 markers selected from group, insulin-like growth factor-binding protein 7, metallopeptidase inhibitor 2, neutrophil elastase, hyaluronic acid, alpha-1 antitrypsin, serum amyloid p component, β-2 glycoprotein, NGAL, KIM-1, Cystatin C, serum creatinine, L-FABP, IL-18, pi-GST, alph-GST, and Clusterin.

In yet other embodiments, the present devices can be used for quantitatively detecting at least 2, 3 or all 4 markers selected from group, insulin-like growth factor-binding protein 7, metallopeptidase inhibitor 2, neutrophil elastase, and hyaluronic acid. For example, the present devices can be used for quantitatively detecting 2 markers, such as: insulin-like growth factor-binding protein 7 and metallopeptidase inhibitor 2; insulin-like growth factor-binding protein 7 and neutrophil elastase; insulin-like growth factor-binding protein 7 and hyaluronic acid; metallopeptidase inhibitor 2 and neutrophil elastase; metallopeptidase inhibitor 2 and hyaluronic acid; neutrophil elastase and hyaluronic acid. The present devices can be used for quantitatively detecting 3 markers, such as: insulin-like growth factor-binding protein 7, metallopeptidase inhibitor 2 and neutrophil elastase; insulin-like growth factor-binding protein 7, metallopeptidase inhibitor 2, and hyaluronic acid; insulin-like growth factor-binding protein 7, neutrophil elastase, and hyaluronic acid; metallopeptidase inhibitor 2, neutrophil elastase and hyaluronic acid. The present devices can be used for quantitatively detecting all 4 markers: insulin-like growth factor-binding protein 7, metallopeptidase inhibitor 2, neutrophil elastase, and hyaluronic acid.

The following Table 1 provides a list of further exemplary biomarkers for kidney injury, renal status and/or risk stratification. In the Table 1, the “recommended name” for the biomarker precursor from the Swiss-Prot “UniProtKB” database, and for most polypeptide biomarkers the Swiss-Prot entry number for the human precursor. In the event that the assay detects a complex, the Swiss Prot entry is listed for each member of the complex.

TABLE 1

| | Swiss- | | Swiss- |
|---|---|---|---|
| Preferred Name | Prot: | Preferred Name | Prot: |
| 60 kDa heat shock protein, mitochondrial | P10809 | 72 kDa type IV collagenase | P08253 |
| 72 kDa type IV collagenase: Metalloproteinase | P08253 | 72 kDa type IV collagenase: Metalloproteinase | P08253 |
| inhibitor 1 complex | P01033 | inhibitor 2 complex | P16035 |
| 72 kDa type IV collagenase: Metalloproteinase | P08253 | Adiponectin | Q15848 |
| inhibitor 4 complex | Q99727 | | |
| Advanced glycosylation end product-specific | Q15109 | Agouti-related protein | 000253 |
| receptor | | | |
| Alkaline phosphatase, tissue-nonspecific | P05186 | Alpha-1-antitrypsin | P01009 |
| isozyme | | | |
| Alpha-1-antitrypsin: Neutrophil elastase | P01009 | Alpha-1-antitrypsin: Plasminogen complex | P01009 |
| complex | P08246 | | P00747 |
| Alpha-2 macroglobulin | P01023 | Alpha-2-HS-glycoprotein | P02765 |
| Alpha-fetoprotein | P02771 | Amphiregulin | P15514 |
| Amyloid Beta 40 | P05067 | Amyloid Beta 42 | P05067 |
| Angiogenin | P03950 | Angiopoietin-1 | Q15389 |
| Angiopoietin-1 receptor | Q02763 | Angiopoietin-2 | 015123 |
| Angiopoietin-related protein 3 | Q9Y5C1 | Angiopoietin-related protein 4 | Q9BY76 |
| Angiopoietin-related protein 6 | Q8NI99 | Anti-Cathepsin-G (ANCA) | |
| Antileukoproteinase | P03973 | Apolipoprotein A-I | P02647 |
| Apolipoprotein A-II | P02652 | Apolipoprotein B-100 | P04114 |
| Apolipoprotein C-III | P02656 | Apolipoprotein E | P02649 |
| Apolipoprotein(a) | P08519 | Appetite-regulating hormone | Q9UBU3 |
| Aspartate aminotransferase, cytoplasmic | P17174 | Bactericidal permeability-increasing protein | P17213 |
| Bcl2 antagonist of cell death | Q92934 | Beta-2-glycoprotein 1 | P02749 |
| Beta-2-microglobulin | P61769 | Beta-nerve growth factor | P01138 |
| Betacellulin | P35070 | Bone morphogenetic protein 7 | P18075 |
| Brain-derived neurotrophic factor | P23560 | C-C motif chemokine 1 | P22362 |
| C-C motif chemokine 13 | Q99616 | C-C motif chemokine 15 | Q16663 |
| C-C motif chemokine 17 | Q92583 | C-C motif chemokine 18 | P55774 |
| C-C motif chemokine 19 | Q99731 | C-C motif chemokine 2 | P13500 |
| C-C motif chemokine 20 | P78556 | C-C motif chemokine 21 | 000585 |
| C-C motif chemokine 22 | 000626 | C-C motif chemokine 23 | P55773 |
| C-C motif chemokine 24 | 000175 | C-C motif chemokine 26 | Q9Y258 |
| C-C motif chemokine 27 | Q9Y4X3 | C-C motif chemokine 3 | P10147 |
| C-C motif chemokine 4 | P13236 | C-C motif chemokine 5 | P13501 |
| C-C motif chemokine 7 | P80098 | C-C motif chemokine 8 | P80075 |
| C-Peptide | P01308(aa | C-reactive protein | P02741 |
| C-X-C motif chemokine 10 | P02778 | C-X-C motif chemokine 11 | 014625 |
| C-X-C motif chemokine 13 | 043927 | C-X-C motif chemokine 16 | Q9H2A7 |
| C-X-C motif chemokine 2 | P19875 | C-X-C motif chemokine 5 | P42830 |
| C-X-C motif chemokine 6 | P80162 | C-X-C motif chemokine 9 | Q07325 |
| Cadherin-1 | P12830 | Cadherin-16 | 075309 |
| Cadherin-3 | P22223 | Cadherin-5 | P33151 |
| Calbindin | P05937 | Calcitonin | P01258 |
| Calcitonin (Procalcitonin) | P01258- | Cancer Antigen 15-3 | |
| Cancer Antigen 19-9 | NA | Carbonic anhydrase 9 | Q16790 |
| Carcinoembryonic antigen-related cell | P13688 | Carcinoembryonic antigen-related cell adhesion | P06731 |
| Caspase-1 | P29466 | Caspase-3, active | P42574 |
| Caspase-8 | Q14790 | Caspase-9 | P55211 |
| Cathepsin B | P07858 | Cathepsin D | P07339 |
| Cathepsin S | P25774 | CD40 ligand | P29965 |
| CD44 antigen | P16070 | Cellular tumor antigen p53 | P04637 |
| Choriogonadotropin subunit beta | P01233 | Ciliary neurotrophic factor | P26441 |
| Clusterin | P10909 | Coagulation factor VII | P08709 |
| Collagenase 3 | P45452 | Complement C3 | P01024 |
| Complement C4-B | POCOL5 | Complement C5 | P01031 |
| Complement factor H | P08603 | Corticotropin | P01189(aa |
| Cortisol | NA | Creatine Kinase-MB | P12277 |
| Creatinine | NA | Cyclin-dependent kinase inhibitor 1 | P38936 |
| Cystatin-C | P01034 | Cytochrome c | P99999 |
| DDRGK domain-containing protein 1 | Q96HY6 | Dipeptidyl peptidase 4 | P27487 |
| E-selectin | P16581 | Endoglin | P17813 |
| Endostatin | P39060(aa | Endothclial protein C receptor | Q9UNN8 |
| Endothelin-1 | P05305 | Eotaxin | P51671 |
| Epidermal growth factor receptor | P00533 | Epiregulin | 014944 |
| Epithelial cell adhesion molecule | P16422 | Erythropoietin | P01588 |
| Erythropoietin receptor | P19235 | Fatty acid-binding protein, heart | P05413 |
| Fatty acid-binding protein, intestinal | P12104 | Fatty acid-binding protein, liver | P07148 |
| Ferritin | P02792 | Fibrinogen | P02671 |
| Fibroblast growth factor 19 | 095750 | Fibroblast growth factor 21 | Q9NSA1 |
| Fibroblast growth factor 23 | Q9GZV9 | Fibronectin | P02751 |
| Follistatin | P19883 | Follitropin | P01215 |
| Follitropin subunit beta | P01225 | Fractalkine | P78423 |
| Galectin-3 | P17931 | Gastric inhibitory polypeptide | P09681 |
| Glial cell line-derived neurotrophic factor | P39905 | Glial fibrillary acidic protein | P14136 |
| Glucagon | P01275 | Glucagon-like peptide 1 | P01275(aa |
| Glutathione S-transferase A1 | P08263 | Glutathione S-transferase P | P09211 |
| Granulocyte colony-stimulating factor | P09919 | Granulocyte-macrophage colony-stimulating | P04141 |
| Granzyme B | P10144 | GranzymeM | P51124 |
| Growth-regulated alpha protein | P09341 | Haptoglobin | P00738 |
| Heat shock 70 kDa protein 1 | P08107 | Heat shock protein beta-1 | P04792 |
| Heat shock protein beta-1 (phospho SER78 I | P04792 | Heat shock protein HSP 90-alpha | P07900 |
| Heme oxygenase 1 | P09601 | Heparan Sulfate | |
| Heparin-binding EGF-like growth factor | Q99075 | Heparin-binding growth factor 1 | P05230 |
| Heparin-binding growth factor 2 | P09038 | Hepatitis A virus cellular receptor 1 | 043656 |
| Hepatocyte growth factor | P14210 | Hepatocyte growth factor receptor | P08581 |
| Hyaluronic acid | NA | Hypoxia-inducible factor 1 alpha | Q16665 |
| Immunoglobulin A | NA | Immunoglobulin E | |
| Immunoglobulin M | NA | Immunoglogulin G1 | |
| Immunoglogulin G2 | NA | Immunoglogulin G3 | |
| Immunoglogulin G4 | NA | Insulin | P01308 |
| Insulin receptor substrate 1 | P35568 | Insulin-like growth factor 1 receptor | P08069 |
| Insulin-like growth factor IA | P01343 | Insulin-like growth factor-binding protein 1 | P08833 |
| Insulin-like growth factor-binding protein 2 | P18065 | Insulin-like growth factor-binding protein 3 | P17936 |
| Insulin-like growth factor-binding protein 4 | P22692 | Insulin-like growth factor-binding protein 5 | P24593 |
| Insulin-like growth factor-binding protein 6 | P24592 | Insulin-like growth factor-binding protein 7 | Q16270 |
| Intercellular adhesion molecule 1 | P05362 | Intercellular adhesion molecule 2 | P13598 |
| Intercellular adhesion molecule 3 | P32942 | Interferon alpha-2 | P01563 |
| Interferon gamma | P01579 | Interleukin-1 alpha | P01583 |
| Interleukin-1 beta | P01584 | Interleukin-1 receptor antagonist protein | P18510 |
| Interleukin-1 receptor type I | P14778 | Interleukin-1 receptor type II | P27930 |
| Interleukin-10 | P22301 | Interleukin-11 | P20809 |
| Interleukin-12 | P29459 | Interleukin-12 subunit beta | P29460 |
| Interleukin-13 | P35225 | Interleukin-15 | P40933 |
| Interleukin-17A | Q16552 | Interleukin-18 | Q14116 |
| Interleukin-2 | P60568 | Interleukin-2 receptor alpha chain | P01589 |
| Interleukin-20 | Q9NYY1 | Interleukin-21 | Q9IIBE4 |
| Interleukin-23 | Q9NPF7 | Interleukin-28A | Q8IZJO |
| Interleukin-29 | Q8IU54 | Interleukin-3 | P08700 |
| Interleukin-33 | 095760 | Interleukin-4 | P05112 |
| Interleukin-4 receptor alpha chain | P24394 | Interleukin-5 | P05113 |
| Interleukin-6 | P05231 | Interleukin-6 receptor subunit alpha | P08887 |
| Interleukin-6 receptor subunit beta | P40189 | Interleukin-7 | P13232 |
| Interleukin-8 | P10145 | Interleukin-9 | P15248 |
| Interstitial collagenase | P03956 | Interstitial collagenase: Metalloproteinase | P03956 |
| | | inhibitor 2 complex | P16035 |
| Involucrin | P07476 | Islet amyloid polypeptide | P10997 |
| Keratin, type I cytoskeletal19 (aa311-367) | P08727 | Keratin, type II cytoskeletal1; type1 | P04264 |
| | | cytoskeletallO (Keratin-1, -10 mix) | P13645 |
| Keratin, type II cytoskeletal 6 (6A, -6B, -6C | P02538 | Kit ligand | P21583 |
| mix) | P04259 | | |
| | P48668 | | |
| Lactotransferrin | P02788 | Leptin | P41159 |
| Leukemia inhibitory factor | P15018 | Lipopolysaccharide (serotypes -K, -O) | |
| Lutropin | P01215 | Lutropin subunit beta | P01229 |
| | P01229 | | |
| Lymphatic vessel endothelial hyaluronic acid | Q9Y5Y7 | Lymphotactin | P47992 |
| receptor 1 | | | |
| Lymphotoxin-alpha | P01374 | Lysozyme C | P61626 |
| Macrophage colony-stimulating factor 1 | P09603 | Macrophage metalloelastase | P39900 |
| Macrophage migration inhibitory factor | P14174 | Malondialdehyde-modified low-density | |
| | | lipoprotein | |
| Matrilysin | P09237 | Matrix metalloproteinase-9 | P14780 |
| Matrix metalloproteinase-9: Metalloproteinase | P14780 | Matrix metalloproteinase-9: Metalloproteinase | P14780 |
| inhibitor 2 complex | P16035 | inhibitor 3 complex | P35625 |
| Metalloproteinase inhibitor 1 | P01033 | Metalloproteinase inhibitor 2 | P16035 |
| Metalloproteinase inhibitor 3 | P35625 | Metalloproteinase inhibitor 4 | Q99727 |
| Midkine | P21741 | Mix of Growth-regulated alpha, beta, and | P09341 |
| | | gamma proteins | P19875 |
| | | | P19876 |
| Monocyte differentiation antigen CD14 | P08571 | Mucin-16 | Q8WXI7 |
| Myeloid differentiation primary response | Q99836 | Myeloperoxidase | P05164 |
| protein MyD88 | | | |
| Myoglobin | P02144 | Neprilysin | P08473 |
| Netrin-1 | 095631 | Neural cell adhesion molecule 1 | P13591 |
| Neuronal cell adhesion molecule | Q92823 | Neutrophil collagenase | P22894 |
| Neutrophil elastase | P08246 | Neutrophil gelatinase-associated lipocalin | P80188 |
| NF-kappa-B inhibitor alpha | P25963 | Nidogen-1 | P14543 |
| Nitric oxide synthase, inducible | P35228 | NT-pro-BNP | P16860 |
| Osteocalcin | P02818 | Osteopontin | P10451 |
| Oxidized low-density lipoprotein receptor 1 | P78380 | P-selectin | P16109 |
| P-selectin glycoprotein ligand 1 | Q14242 | Pancreatic prohormone | P01298 |
| Pappalysin-1 | Q13219 | Parathyroid hormone | P01270 |
| Peptide YY | P10082 | Pigment epithelium-derived factor | P36955 |
| Placenta growth factor | P49763 | Plasminogen activator inhibitor 1 | P05121 |
| Platelet basic protein | P02775 | Platelet endothelial cell adhesion molecule | P16284 |
| Platelet factor 4 | P02776 | Platelet-derived growth factor A | P04085 |
| | | | P01127 |
| Platelet-derived growth factor subunit A | P04085 | Platelet-derived growth factor subunit B | P01127 |
| (dimer) | | (dimer) | |
| Poly [ADP-ribose] polymerase 1 (cleaved) | P09874 | Pro-epidermal growth factor | P01133 |
| Pro-Interleukin-1 beta | P01584- | Pro-interleukin-16 | Q14005 |
| | Pro | | |
| Prolactin | P01236 | Prostate-specific antigen | P07288 |
| Prostatic acid phosphatase | P15309 | Protein NOV homolog | P48745 |
| Protein S100-A12 | P80511 | Protein S1OO-B | P04271 |
| Protransforming growth factor alpha | P01135 | Renin | P00797 |
| Resistin | Q9HD89 | Serum albumin | P02768 |
| Serum amyloid A protein | P02735 | Serum amyloid P-component | P02743 |
| Sex hormone-binding globulin | P04278 | SL cytokine | P49771 |
| Somatotropin | P01241 | Stromal cell-derived factor 1 | P48061 |
| Stromelysin-1 | P08254 | Stromelysin-1: Metalloproteinase inhibitor 2 | P08254 |
| | | complex | P16035 |
| Stromelysin-2 | P09238 | Tenascin | P24821 |
| Thrombomodulin | P07204 | Thrombopoietin | P40225 |
| Thrombospondin-1 | P07996 | Thrombospondin-2 | P35442 |
| Thymic stromallymphopoietin | Q969D9 | Thyrotropin | P01215 |
| | | | P01222 |
| Thyroxine-binding globulin | P05543 | Tissue factor | P13726 |
| Tissue-type plasminogen activator | P00750 | Transforming growth factor beta-1 | P01137 |
| Transforming growth factor beta-2 | P61812 | Transforming growth factor beta-3 | P10600 |
| Transmembrane glycoprotein NMB | Q14956 | Transthyretin | P02766 |
| Trefoil factor 3 | Q07654 | Tubulointerstitial nephritis antigen | Q9UJW2 |
| Tumor necrosis factor | P01375 | Tumor necrosis factor ligand superfamily | P50591 |
| | | member 10 | |
| Tumor necrosis factor ligand superfamily | 014788 | Tumor necrosis factor ligand superfamily | P48023 |
| member 11 | | member 6 | |
| Tumor necrosis factor receptor superfamily | 014763 | Tumor necrosis factor receptor superfamily | 000300 |
| member 10B | | member 11B | |
| Tumor necrosis factor receptor superfamily | P19438 | Tumor necrosis factor receptor superfamily | P20333 |
| member 1A | | member 1B | |
| Tumor necrosis factor receptor superfamily | P25942 | Tumor necrosis factor receptor superfamily | P25445 |
| member 5 | | member 6 | |
| Tumor necrosis factor receptor superfamily | P28908 | Urokinase plasminogen activator surface | Q03405 |
| member 8 | | receptor | |
| Urokinase-type plasminogen activator | P00749 | Vascular cell adhesion protein 1 | P19320 |
| Vascular endothelial growth factor A | P15692 | Vascular endothelial growth factor D | 043915 |
| Vascular endothelial growth factor receptor 1 | P17948 | Vascular endothelial growth factor receptor 2 | P35968 |
| Vascular endothelial growth factor receptor 3 | P35916 | Versican core protein | P13611 |
| Vitamin D-binding protein | P02774 | Vitamin K-dependent protein C | P04070 |
| von Willebrand Factor | P04275 | WAP four-disulfide core domain protein 2 | Q14508 |

Included in Table 1 above are a number of proteins which exist in one form as type-I, type-II, or GPI-anchored membrane proteins. Typically, such membrane proteins comprise a substantial extracellular domain, some or all of which can be detected as soluble forms present in aqueous samples such as blood, serum, plasma, urine, etc., either as cleavage products or as splice variants which delete an effective membrane spanning domain. These membrane proteins include Swiss-Prot entry numbers 014788, 014944, 075309, P00797, P05186, P08473, P13688, P15514, P22223, P27487, P35070, Q03405, Q14956, Q16790, Q99075, Q9Y5Y7Q15109, Q02763, P17213, P12830, P33151, P06731, P29965, P16070, Q9H2A7, P17813, Q9UNN8, P00533, P16422, P19235, P16581, P78423, 043656, P08581, P08069, P05362, P13598, P32942, P14778, P27930, P01589, P24394, P08887, P40189, P21583, P09603, P08571, Q8VVXI7, P13591, Q92823, P78380, P16284, PO1133, P15309, PO1135, P16109, Q14242, P49771, P07204, P13726, P01375, P50591, P48023, O14763, P19438, P20333, P25942, P25445, P28908, P19320, P17948, and P35968. Preferred assays detect soluble forms of these biomarkers.

The present test device can be used for quantitatively detecting analytes at any suitable level, concentration or range of level or concentration. In some embodiments, the present test device can be used for quantitatively detecting analytes, wherein at least one or some of the analytes have a concentration ranging from about 1 pg/ml to about 1 μg/ml, e.g., about 1 pg/ml, 10 pg/ml, 100 pg/ml, 1 ng/ml, 2 ng/ml, 3 ng/ml, 3.5 ng/ml, 4 ng/ml, 5 ng/ml, 6 ng/ml, 7 ng/ml, 8 ng/ml, 9 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 950 ng/ml, or higher. In other embodiments, the present test device can be used for quantitatively detecting analytes, wherein each of the analytes has a concentration ranging from about 1 pg/ml to about 1 μg/ml, e.g., about 1 pg/ml, 10 pg/ml, 100 pg/ml, 1 ng/ml, 2 ng/ml, 3 ng/ml, 3.5 ng/ml, 4 ng/ml, 5 ng/ml, 6 ng/ml, 7 ng/ml, 8 ng/ml, 9 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 950 ng/ml, or higher.

The present test device can be used for quantitatively detecting analytes with any desired or intended precision. In some embodiments, the present test device can be used for quantitatively detecting analytes, wherein the amount of at least one analyte, some analytes, or each of the analytes is determined with a CV ranging from about 0.1% to about 10%. Preferably, at least one analyte, some analytes, or each of the analytes has a concentration ranging from about 1 pg/ml to about 1 μg/ml, e.g., about 1 pg/ml, 10 pg/ml, 100 pg/ml, 1 ng/ml, 2 ng/ml, 3 ng/ml, 3.5 ng/ml, 4 ng/ml, 5 ng/ml, 6 ng/ml, 7 ng/ml, 8 ng/ml, 9 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 950 ng/ml, or higher.

The present test device can further comprise a liquid container. The liquid container can comprise any suitable liquid and/or reagent. For example, the liquid container can comprise a developing liquid, a wash liquid and/or a labeled reagent

The present test device can further comprise machine-readable information, e.g., a barcode. The barcode can comprise any suitable information. In some embodiments, the barcode comprises lot specific information of the test device, e.g., lot number of the test device. In other embodiments, the machine-readable information is comprised in a storage medium, e.g., a (radio-frequency identification) RFID device. The RFID device can comprise any suitable information. For example, the RFID device comprises lot specific information, information on a liquid control or information to be used for quality control purpose.

In some embodiments, a fluorescent conjugate comprising a biological reagent and a fluorescent molecule is used to generate a detectable signal at the test locations. In this case, the fluorescent conjugate and/or the test device can further comprise a means for impeding phototoxic degradation of the biological reagent or impeding nonspecific binding of the fluorescent conjugate to the test device or a non-analyte moiety. Any suitable means or substances can be used to impede phototoxic degradation of the biological reagent. See. e.g., U.S. Pat. Nos. 6,544,797 and 7,588,908. For example, the means for impeding phototoxic degradation of the biological reagent can comprise a cross-linking substance having a long molecular distance, whereby the cross-linking substance links the fluorescent molecule and the biological reagent. In other examples, a protein; a quencher of singlet oxygen; a quencher of a free radical; a system for depleting oxygen; or a combination thereof can be used to impede phototoxic degradation of the biological reagent.

Any suitable means or substances can be used to impede nonspecific binding of the fluorescent conjugate. For example, the means for impeding nonspecific binding of the fluorescent conjugate comprises PEG or PEO bound to the fluorescent conjugate.

The test reagent(s) and/or the labeled reagent(s) can be any suitable substances. For example, the reagents can be inorganic molecules, organic molecules or complexes thereof. Exemplary inorganic molecules can be ions such as sodium, potassium, magnesium, calcium, chlorine, iron, copper, zinc, manganese, cobalt, iodine, molybdenum, vanadium, nickel, chromium, fluorine, silicon, tin, boron or arsenic ions. Exemplary organic molecules can be an amino acid, a peptide, a protein, e.g., an antibody or receptor, a nucleoside, a nucleotide, an oligonucleotide, a nucleic acid, e.g., DNA or RNA, a vitamin, a monosaccharide, an oligosaccharide, a carbohydrate, a lipid, an aptamer and a complex thereof.

Exemplary amino acids can be a D- or a L-amino-acid. Exemplary amino acids can also be any building blocks of naturally occurring peptides and proteins including Ala (A), Arg (R), Asn (N), Asp (D), Cys (C), Gln (Q), Glu (E), Gly (G), His (H), Ile (I), Leu (L), Lys (K), Met (M), Phe (F), Pro (P) Ser (S), Thr (T), Trp (W), Tyr (Y) and Val (V).

Any suitable proteins or peptides can be used as the test reagent(s) and/or the labeled reagent(s). For example, enzymes, transport proteins such as ion channels and pumps, nutrient or storage proteins, contractile or motile proteins such as actins and myosins, structural proteins, defense protein or regulatory proteins such as antibodies, hormones and growth factors can be used. Proteineous or peptidic antigens can also be used.

Any suitable nucleic acids, including single-, double and triple-stranded nucleic acids, can be used as the test reagent(s) and/or the labeled reagent(s). Examples of such nucleic acids include DNA, such as A-, B- or Z-form DNA, and RNA such as mRNA, tRNA and rRNA.

Any suitable nucleosides can be can be used as the test reagent(s) and/or the labeled reagent(s). Examples of such nucleosides include adenosine, guanosine, cytidine, thymidine and uridine. Any nucleotides can be used as the reagents on the test device. Examples of such nucleotides include AMP, GMP, CMP, UMP, ADP, GDP, CDP, UDP, ATP, GTP, CTP, UTP, dAMP, dGMP, dCMP, dTMP, dADP, dGDP, dCDP, dTDP, dATP, dGTP, dCTP and dTTP.

Any suitable vitamins can be used as test reagent(s) and/or the labeled reagent(s). For example, water-soluble vitamins such as thiamine, riboflavin, nicotinic acid, pantothenic acid, pyridoxine, biotin, folate, vitamin B₁₂ and ascorbic acid can be used. Similarly, fat-soluble vitamins such as vitamin A, vitamin D, vitamin E, and vitamin K can be used.

Any suitable monosaccharides, whether D- or L-monosaccharides and whether aldoses or ketoses, can be used as the test reagent(s) and/or the labeled reagent(s). Examples of monosaccharides include triose such as glyceraldehyde, tetroses such as erythrose and threose, pentoses such as ribose, arabinose, xylose, lyxose and ribulose, hexoses such as allose, altrose, glucose, mannose, gulose, idose, galactose, talose and fructose and heptose such as sedoheptulose.

Any suitable lipids can be used as the test reagent(s) and/or the labeled reagent(s). Examples of lipids include triacylglycerols such as tristearin, tripalmitin and triolein, waxes, phosphoglycerides such as phosphatidylethanolamine, phosphatidylcholine, phosphatidylserine, phosphatidylinositol and cardiolipin, sphingolipids such as sphingomyelin, cerebrosides and gangliosides, sterols such as cholesterol and stigmasterol and sterol fatty acid esters. The fatty acids can be saturated fatty acids such as lauric acid, myristic acid, palmitic acid, stearic acid, arachidic acid and lignoceric acid, or can be unsaturated fatty acids such as palmitoleic acid, oleic acid, linoleic acid, linolenic acid and arachidonic acid.

In one specific embodiment, analytes to be detected comprise or are antigens, the test reagent(s) and/or the labeled reagent(s) comprises or is an antibody. Preferably, the antibody or antibodies specifically bind to the analyte(s). In one example, the test device is used in a sandwich assay format, in which an antibody is used as a test reagent at a test location, and another binding reagent having a detectable label is used to form a labeled binding reagent-analyte-test reagent or antibody sandwich at a test location to generate a readout signal. Alternatively, a binding reagent is used as a reagent at a test location, and an antibody have a detectable label is used to form a labeled antibody-analyte-binding reagent sandwich at the test location to generate a readout signal.

In some embodiments, the sandwich assay uses antibodies as the test reagent(s) and the labeled reagent(s). In one example, an assay uses the same labeled antibody to bind to the multiple analytes. In another example, an assay uses multiple labeled antibodies, each of the labeled antibodies binding to a different analyte. In still another example, an assay uses the same antibody at multiple or all test locations to bind to the multiple analytes. In yet another example, an assay uses multiple antibodies at multiple or all test locations, each of the antibodies binding to a different analyte. Certain combinations can also be used. For example, an assay uses the same labeled antibody to bind to the multiple analytes and multiple antibodies at multiple or all test locations, each of the antibodies at the test locations binding to a different analyte. In another example, an assay uses multiple labeled antibodies, each of the labeled antibodies binding to a different analyte, and a single antibody at the test locations to binding to the multiple analytes. In still another example, an assay uses different labeled antibodies to bind to different analytes and different antibodies at the test locations to bind to different analytes.

The test device can also be used in a competition assay format. In one example, a test reagent, e.g., an antibody, can be used as a capture reagent at a test location. An analyte or analyte analog having a detectable label, either added in a liquid or previously dried on the test device and redissolved or resuspended by a liquid, will compete with an analyte in a sample to bind to the capture reagent at the test location. Typically, different capture reagents. e.g., different antibodies, are used at different test locations to bind to different analytes. In another example, an analyte or analyte analog is used as a capture reagent at the test location. A labeled reagent, e.g., an antibody having a detectable label, is either added in a liquid or previously dried on the test device and redissolved or resuspended by a liquid. An analyte in a sample will compete with the analyte or analyte analog at the test location for binding to the labeled reagent, e.g., an antibody, having a detectable label. Typically, different analytes or analyte analogs are used at different test locations to compete with different analytes for binding to the different labeled reagents.

Antibodies used in the immunoassays described herein preferably specifically bind to a target analyte, e.g., a kidney injury marker of the present invention. The term “specifically binds” is not intended to indicate that an antibody binds exclusively to its intended target since, as noted above, an antibody binds to any polypeptide displaying the epitope(s) to which the antibody binds. In some cases, an antibody “specifically binds” if its affinity for its intended target is about 5-fold greater when compared to its affinity for a non-target molecule which does not display the appropriate epitope(s). Preferably the affinity of the antibody may be at least about 5 fold, preferably 10 fold, more preferably 25-fold, even more preferably 50-fold, and most preferably 100-fold or more, greater for a target molecule than its affinity for a non-target molecule. In preferred embodiments, preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹² M⁻¹.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

Numerous publications discuss the use of phage display technology to produce and screen libraries of polypeptides for binding to a selected analyte. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

While the present application describes antibody-based binding assays in detail, alternatives to antibodies as binding species in assays are well known in the art. These include receptors for a particular target, aptamers, etc. Aptamers are oligonucleic acid or peptide molecules that bind to a specific target molecule. Aptamers are usually created by selecting them from a large random sequence pool, but natural aptamers also exist. High-affinity aptamers containing modified nucleotides conferring improved characteristics on the ligand, such as improved in vivo stability or improved delivery characteristics. Examples of such modifications include chemical substitutions at the ribose and/or phosphate and/or base positions, and may include amino acid side chain functionalities.

In some embodiments, the present invention provides for a test device wherein a liquid has moved laterally along the test device to generate a detectable signal at the test locations.

The present invention also provides for a kit for quantitatively detecting multiple analytes in a sample, which kit comprises a test device as described above. In some embodiments, the kit can further comprise an instruction for using the test device to quantitatively detect multiple analytes in a sample, and/or means for obtaining and/or processing the sample to be tested.

### C. Methods for Quantitatively Detecting Multiple Analytes in a Sample

In another aspect, the present invention provides a method for quantitatively detecting multiple analytes in a sample, which method comprises: a) contacting a liquid sample with the test device described above, wherein the liquid sample is applied to a site of the test device upstream of the test locations; b) transporting multiple analytes, if present in the liquid sample, and a labeled reagent to the test locations; and c) assessing a detectable signal at the test locations to determine the amounts of the multiple analytes in the sample.

The present methods can be used to determine amounts of multiple analytes with desired or intended precision. Typically, the amount of each of the multiple analytes is determined with a precision, or coefficient of variation (CV), at about 30% or less, at analyte level(s) or concentration(s) that encompasses one or more desired threshold values of the analyte(s), and/or at analyte level(s) or concentration(s) that is below, at about low end, within, at about high end, and/or above one or more desired reference ranges of the analyte(s).

In some embodiments, it is often desirable or important to have higher precision, e.g., CV less than 30%, 25%, 20%, 15%, 10%, 9%, 8%, 7%, 6%, 5%, 4%, 3%, 2%, 1%, 0.5%, 0.1%, or smaller, at the desired analyte level(s) or concentration(s).

In other embodiments, it is often desirable or important that the analytes are quantified with a desired or required CV at analyte level(s) or concentration(s) that is substantially lower than, at about, or at, and/or substantially higher than the desired or required threshold values of the analyte(s). The precision or CV standard can be applied to the assays wherein the amount of each analyte is determined and compared to its corresponding threshold value individually. For example, each of the analytes can be quantified with a desired or required CV at analyte level or concentration that is substantially lower than the desired or required threshold values of the analyte. In another example, each of the analytes can be quantified with a desired or required CV at analyte level or concentration that is at about, or at, the desired or required threshold value of the analyte. In still another example, each of the analytes can be quantified with a desired or required CV at analyte level or concentration that is substantially higher than the desired or required threshold values of the analyte. In yet another example, each of the analytes can be quantified with a desired or required CV at analyte level or concentration range that is from substantially lower than to substantially higher than the desired or required threshold values of the analyte. The multiple analytes can be quantified with the same level or different levels of CV, or with the same range or different ranges of CV. The precision or CV standard can also be applied to the assays wherein the amounts of the multiple analytes are quantified and converted into a composite amount and the composite analyte amount is compared to its corresponding composite threshold value.

In still other embodiments, it is often desirable or important that the analytes are quantified with a desired or required CV at analyte level(s) or concentration(s) that is substantially lower than the low end of the reference range(s), that encompasses a portion or the entire reference range(s), and/or that is substantially higher than the high end of the reference range(s). The precision or CV standard can be applied to the assays wherein the amount of each analyte is determined and compared to its corresponding reference range individually. For example, each of the analytes can be quantified with a desired or required CV at analyte level or concentration that is substantially lower than the low end of the reference range of the analyte. In another example, each of the analytes can be quantified with a desired or required CV at analyte level or concentration that encompasses 10%, 20%, 30%, 40%, 50%, 60%, 70%, 80%, 80%, 95%, or the entire reference range of the analyte. In still another example, each of the analytes can be quantified with a desired or required CV at analyte level or concentration that is substantially higher than the high end of the reference range of the analyte. In yet another example, each of the analytes can be quantified with a desired or required CV at analyte level or concentration range that is from substantially lower than the low end of the reference range to substantially higher than the high end of the reference range of the analyte. The multiple analytes can be quantified with the same level or different levels of CV, or with the same range or different ranges of CV. The precision or CV standard can also be applied to the assays wherein the amounts of the multiple analytes are quantified and converted into a composite amount and the composite analyte amount is compared to its corresponding composite reference range.

In some embodiments, the present method can be used for quantitatively detecting analytes, wherein the amount of at least one analyte, some analytes, or each of the analytes is determined with a CV ranging from about 0.1% to about 10%. Preferably, at least one analyte, some analytes, or each of the analytes has a concentration ranging from about 1 pg/ml to about 1 μg/ml, e.g., about 1 pg/ml, 10 pg/ml, 100 pg/ml, 1 ng/ml, 2 ng/ml, 3 ng/ml, 3.5 ng/ml, 4 ng/ml, 5 ng/ml, 6 ng/ml, 7 ng/ml, 8 ng/ml, 9 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 950 ng/ml, or higher.

The liquid sample and the labeled reagent can be premixed to form a mixture and the mixture is applied to the test device. The labeled reagent can be stored and/or used in any suitable manner. For example, the labeled reagent can be stored and/or used in liquid format. Alternatively, the labeled reagent can be stored in a dry format off the device, e.g., in a container, pipette tip, or tube. For example, the labeled reagent can be dried on the surface of the container, pipette tip, or tube. In another example, the labeled reagent can be dried as particles or beads and the particles or beads can be stored in the container, pipette tip, or tube. In use, the dried labeled reagent, either dried on the surface of the container, pipette tip, or tube, or dried as particles or beads, can be dissolved or resuspended by a liquid sample or buffer to form a mixture and the mixture is applied to the test device. In other embodiments, the present method can further comprise a washing step after the mixture is applied to the test device. The washing step can be conducted by any suitable ways. For example, the washing step can comprise adding a washing liquid after the mixture is applied to the test device. In another example, the test device can comprise a liquid container comprising a washing liquid and the washing step comprises releasing the washing liquid from the liquid container. See e.g., U.S. Pat. No. 4,857,453.

The test device can also comprise a dried labeled reagent before use and the dried labeled reagent can be solubilized or resuspended, and transported to the test locations by the liquid sample. In some embodiments, the dried labeled reagent is located downstream from the sample application site, and the dried labeled reagent is solubilized or resuspended, and transported to the test location by the liquid sample. In other embodiments, the dried labeled reagent is located upstream from the sample application site, and the dried labeled reagent is solubilized or resuspended, and transported to the test location by another liquid. In still other embodiments, multiple analytes and/or labeled reagent(s) are solubilized or resuspended, and transported to the test location by the liquid sample alone. In yet other embodiments, multiple analytes and/or labeled reagent(s) are solubilized or resuspended, and transported to the test location by another liquid, or by a combination of the sample liquid and another liquid, e.g., a developing fluid.

The present method can be used for quantitatively detecting multiple analytes in any suitable sample. In some embodiments, the sample is a biological sample or clinical sample. In other embodiments, the sample is a body fluid sample. Exemplary body fluid samples include a whole blood, a serum, a plasma and a urine sample. Other exemplary samples include saliva, semen, stool, sputum, cerebral spinal fluid, tears, mucus, amniotic fluid or the like.

Depending on the assay format and the label used in the method, the detectable signal can be assessed by any suitable methods. For example, when the label is a visual direct label, e.g., a gold or latex particle label, the detectable signal can be assessed by naked eyes without using any instrument. In other examples, the detectable signal is often or typically assessed by a reader. In many cases, a reader is used to assess the detectable signal regardless whether the detectable signal can be assessed by naked eyes or not. For example even if a visual direct label is used, the detectable signal is often or typically assessed by a reader for quantitatively detecting the analytes.

In some embodiments, the detectable signal is a fluorescent signal and the fluorescent signal is assessed by a fluorescent reader. Depending on the assay format and the fluorescent label used in the method, any suitable fluorescent reader can be used. For example, the fluorescent reader can be a laser based or a light emitting diode (LED) based fluorescent reader.

The fluorescent reader can illuminate at any suitable angle relative to the surface of the test device to excite the fluorescent label at the test locations and/or can detect the fluorescent light at any suitable angle relative to the surface of the test device. In some embodiments, the fluorescent reader illuminates at an angle substantially normal, or normal, to the surface of the test device to excite the fluorescent label at the test locations and/or detects the fluorescent light at an angle substantially normal, or normal, to the surface of the test device. In other embodiments, the surface for detection of the fluorescent light in the fluorescent reader is substantially parallel, or parallel, to the surface of the test device. In still other embodiments, the surface for detection of the fluorescent light in the fluorescent reader is not parallel to the surface of the test device. A light source and a photodetector can be positioned at the same side or different sides of the test device.

An illumination system of the reader can scan any suitable or desired size or defined area of the test and/or control locations to detect the detectable or fluorescent signal. In some embodiments, at least one, some or each of the test locations comprises a capture region characterized by a first dimension transverse to the lateral flow direction and a second dimension parallel to the lateral flow direction, and the reader comprises an illumination system operable to focus a beam of light onto an area of the test and/or control locations having at least one surface dimension at most equal to smallest of the first and second dimensions of the test and/or control locations.

The reader can comprise a single or multiple photodetectors. The detectable signal can be measured at any suitable or desired time point(s). In some embodiments, the detectable signal is measured before the detectable signal reaches its equilibrium. In other embodiments, the detectable signal is measured after the detectable signal reaches its equilibrium. In still other embodiments, the detectable signal is measured at a preset time after the sample is added to the test device.

The present methods can further comprise comparing the amounts of the multiple analytes to a single threshold, multiple thresholds or a reference range, e.g., a normal range, a disease range, a clinical range, or a reference range based on calibrated or uncalibrated analyte levels or concentrations. In some embodiments, the amount of at least one, some or each of the multiple analytes is compared to a single corresponding threshold or multiple corresponding thresholds. In other embodiments, the amounts of the multiple analytes are used to form a composite amount that is compared to a composite threshold or reference range.

The present methods can be used for quantitatively detecting any suitable number of analytes. For example, the present methods can be used for quantitatively detecting 2, 3, 4, 5, 6, 7, 8, 9, 10 or more analytes. The present methods can be used for any suitable purpose. For example, the present can be used for quantitatively detecting multiple analytes that are diagnostic, prognostic, risk assessment, stratification and/or treatment monitoring markers.

The present methods can be used for quantitatively detecting any suitable analytes. Exemplary analytes include markers for diseases or conditions such as infectious diseases, parasitic diseases, neoplasms, diseases of the blood and blood-forming organs, disorders involving the immune mechanism, endocrine, nutritional and metabolic diseases, mental and behavioural disorders, diseases of the nervous system, diseases of the eye and adnexam, diseases of the ear and mastoid process, diseases of the circulatory system, diseases of the respiratory system, diseases of the digestive system, diseases of the skin and subcutaneous tissue, diseases of the musculoskeletal system and connective tissue, diseases of the genitourinary system, pregnancy, childbirth and the puerperium, conditions originating in the perinatal period, congenital malformations, deformations, chromosomal abnormalities, injury, poisoning, consequences of external causes, external causes of morbidity and mortality. (See e.g., International Statistical Classification of Diseases and Related Health Problems, World Health Organization). In some embodiments, the analytes are markers for acute coronary syndrome (ACS), abdominal pain, cerebrovascular injury, kidney injury, e.g., acute kidney injury or chronic kidney disease, or sepsis.

In other embodiments, the present methods can be used for quantitatively detecting any suitable markers for kidney injury. Exemplary markers for kidney injury include insulin-like growth factor-binding protein 7 (or IGFBP7 or FSTL2 or IBP-7 or IGF-binding protein 7 or IGFBP-7 or IGFBP-7v or IGFBPRP1 or IGFBP-rP1 or MAC25 or MAC-25 or MAC 25 or PGI2-stimulating factor or AGM), Metallopeptidase inhibitor 2 (or CSC-21K or Metalloproteinase inhibitor 2 or TIMP-2 or Tissue inhibitor of metalloproteinases 2 or TIMP2 or TIMP 2), Neutrophil elastase (or Bone marrow serine protease or ELA2 or Elastase-2 or HLE or HNE or Human leukocyte elastase or Medullasin or Neutrophil elastase or PMN-E or PMN elastase or SCN1 or ELANE or elastase neutrophil expressed or elastase 2 or neutrophil-derived elastase or granulocyte-derived elastase or polymorphonuclear elastase or leukocyte elastase) and hyaluronic acid (or Hyaluronan or hyaluronate), alpha-1 antitrypsin (A1AT, Alpha-1 protease inhibitor, alpha1AT, serine or cysteine proteinase inhibitor, AAT, PI, PI1, serine or cysteine proteinase inhibitor, clade A, member 1, alpha1AT, A1A, or serpin A1), serum amyloid p component (amyloid P component), β-2 glycoprotein, NGAL, KIM-1, Cystatin C, serum creatinine, L-FABP, IL-18, pi-GST, alph-GST, and Clusterin. In still other embodiments, the present methods can be used for quantitatively detecting at least 2, 3, 4, 5, 6 or all 7 markers selected from group, insulin-like growth factor-binding protein 7, metallopeptidase inhibitor 2, neutrophil elastase, hyaluronic acid, alpha-1 antitrypsin, serum amyloid p component, β-2 glycoprotein, NGAL, KIM-1, Cystatin C, serum creatinine, L-FABP, IL-18, pi-GST, alph-GST, and Clusterin.

In yet other embodiments, the present methods can be used for quantitatively detecting at least 2, 3 or all 4 markers selected from group, insulin-like growth factor-binding protein 7, metallopeptidase inhibitor 2, neutrophil elastase, and hyaluronic acid. For example, the present methods can be used for quantitatively detecting 2 markers, such as: insulin-like growth factor-binding protein 7 and metallopeptidase inhibitor 2; insulin-like growth factor-binding protein 7 and neutrophil elastase; insulin-like growth factor-binding protein 7 and hyaluronic acid; metallopeptidase inhibitor 2 and neutrophil elastase; metallopeptidase inhibitor 2 and hyaluronic acid; neutrophil elastase and hyaluronic acid. The present methods can be used for quantitatively detecting 3 markers, such as: insulin-like growth factor-binding protein 7, metallopeptidase inhibitor 2 and neutrophil elastase; insulin-like growth factor-binding protein 7, metallopeptidase inhibitor 2, and hyaluronic acid; insulin-like growth factor-binding protein 7, neutrophil elastase, and hyaluronic acid; metallopeptidase inhibitor 2, neutrophil elastase and hyaluronic acid. The present methods can be used for quantitatively detecting all 4 markers: insulin-like growth factor-binding protein 7, metallopeptidase inhibitor 2, neutrophil elastase, and hyaluronic acid.

### D. Systems for Quantitatively Detecting Multiple Analytes in a Sample

In another aspect, the present invention provides a system for quantitatively detecting multiple analytes in a sample, which system comprises: a) a test device described above; and b) a reader that comprises a light source and a photodetector to detect a detectable signal.

Depending on the assay format and the label used in the assay, any suitable reader can be used, e.g., a fluorescent reader. Depending on the assay format and the fluorescent label used in the method, any suitable fluorescent reader can be used. For example, the fluorescent reader can be a laser based or a light emitting diode (LED) based fluorescent reader.

The fluorescent reader can illuminate at any suitable angle relative to the surface of the test device to excite the fluorescent label at the test locations and/or can detect the fluorescent light at any suitable angle relative to the surface of the test device. In some embodiments, the fluorescent reader illuminates at an angle substantially normal, or normal, to the surface of the test device to excite the fluorescent label at the test locations and/or detects the fluorescent light at an angle substantially normal, or normal, to the surface of the test device. In other embodiments, the surface for detection of the fluorescent light in the fluorescent reader is substantially parallel, or parallel, to the surface of the test device. In still other embodiments, the surface for detection of the fluorescent light in the fluorescent reader is not parallel to the surface of the test device. A light source and a photodetector can be positioned at the same side or different sides of the test device.

An illumination system of the reader can scan any suitable or desired size or defined area of the test and/or control locations to detect the detectable or fluorescent signal. In some embodiments, at least one, some or each of the test locations comprises a capture region characterized by a first dimension transverse to the lateral flow direction and a second dimension parallel to the lateral flow direction, and the reader comprises an illumination system operable to focus a beam of light onto an area of the test and/or control locations having at least one surface dimension at most equal to smallest of the first and second dimensions of the test and/or control locations.

The reader can comprise a single or multiple photodetectors. The detectable signal can be measured at any suitable or desired time point(s). In some embodiments, the detectable signal is measured before the detectable signal reaches its equilibrium. In other embodiments, the detectable signal is measured after the detectable signal reaches its equilibrium. In still other embodiments, the detectable signal is measured at a preset time after the sample is added to the test device.

The present systems can comprise machine-readable information and a reader for detecting the machine-readable information. For example, the test device can comprise machine-readable information, e.g., a barcode, and the reader can comprise a function for detecting the machine-readable information, e.g., a barcode reader. The machine-readable information can be any suitable or desired information, e.g., lot specific information of the test device or the assay, information on a liquid control or information to be used for quality control purpose, etc. In some embodiments, the present system, e.g., the present device, can comprise a barcode that comprises lot specific information of the test device, e.g., lot number of the test device. In other embodiments, the present system can comprise a storage medium, e.g., a RFID device. The RFID device can comprise lot specific information, information on a liquid control or information to be used for quality control purpose. The RFID device can be provided in any suitable ways or locations. For example, an RFID device can be provided as an RFID card with an embedded antenna and an RFID tag. In another example, the RFID device or card can be provided within a package of a plurality of the present devices, or can be provided on the package, but is not made part of a present device. In still another example, the RFID device or card can be provided on any suitable location on a test device, e.g., on the housing of the test device or at any location that is not test locations.

The present systems can be used for quantitatively detecting any suitable number of analytes. For example, the present systems can be used for quantitatively detecting 2, 3, 4, 5, 6, 7, 8, 9, 10 or more analytes. The present systems can be used for any suitable purpose. For example, the present systems can be used for quantitatively detecting multiple analytes that are diagnostic, prognostic, risk assessment, stratification and/or treatment monitoring markers.

The present systems can be used for quantitatively detecting any suitable analytes. Exemplary analytes include markers for diseases or conditions such as infectious diseases, parasitic diseases, neoplasms, diseases of the blood and blood-forming organs, disorders involving the immune mechanism, endocrine, nutritional and metabolic diseases, mental and behavioural disorders, diseases of the nervous system, diseases of the eye and adnexam, diseases of the ear and mastoid process, diseases of the circulatory system, diseases of the respiratory system, diseases of the digestive system, diseases of the skin and subcutaneous tissue, diseases of the musculoskeletal system and connective tissue, diseases of the genitourinary system, pregnancy, childbirth and the puerperium, conditions originating in the perinatal period, congenital malformations, deformations, chromosomal abnormalities, injury, poisoning, consequences of external causes, external causes of morbidity and mortality. (See e.g., International Statistical Classification of Diseases and Related Health Problems, World Health Organization). In some embodiments, the analytes are markers for acute coronary syndrome (ACS), abdominal pain, cerebrovascular injury, kidney injury, e.g., acute kidney injury, or sepsis

In other embodiments, the present systems can be used for quantitatively detecting any suitable markers for kidney injury. Exemplary markers for kidney injury include insulin-like growth factor-binding protein 7 (or IGFBP7 or FSTL2 or IBP-7 or IGF-binding protein 7 or IGFBP-7 or IGFBP-7v or IGFBPRP1 or IGFBP-rP1 or MAC25 or MAC-25 or MAC 25 or PGI2-stimulating factor or AGM), Metallopeptidase inhibitor 2 (or CSC-21K or Metalloproteinase inhibitor 2 or TIMP-2 or Tissue inhibitor of metalloproteinases 2 or TIMP2 or TIMP 2), Neutrophil elastase (or Bone marrow serine protease or ELA2 or Elastase-2 or HLE or HNE or Human leukocyte elastase or Medullasin or Neutrophil elastase or PMN-E or PMN elastase or SCN1 or ELANE or elastase neutrophil expressed or elastase 2 or neutrophil-derived elastase or granulocyte-derived elastase or polymorphonuclear elastase or leukocyte elastase), hyaluronic acid (or Hyaluronan or hyaluronate), alpha-1 antitrypsin (A1AT, Alpha-1 protease inhibitor, alpha1AT, serine or cysteine proteinase inhibitor, AAT, PI, PI1, serine or cysteine proteinase inhibitor, clade A, member 1, alpha1AT, A1A, or serpin A1), serum amyloid p component (amyloid P component), β-2 glycoprotein, NGAL, KIM-1, Cystatin C, serum creatinine, L-FABP, IL-18, pi-GST, alph-GST, and Clusterin. In still other embodiments, the present systems can be used for quantitatively detecting at least 2, 3, 4, 5, 6 or all 7 markers selected from group, insulin-like growth factor-binding protein 7, metallopeptidase inhibitor 2, neutrophil elastase, hyaluronic acid, alpha-1 antitrypsin, serum amyloid p component, β-2 glycoprotein, NGAL, KIM-1, Cystatin C, serum creatinine, L-FABP, IL-18, pi-GST, alph-GST, and Clusterin.

In yet other embodiments, the present systems can be used for quantitatively detecting at least 2, 3 or all 4 markers selected from group, insulin-like growth factor-binding protein 7, metallopeptidase inhibitor 2, neutrophil elastase, and hyaluronic acid. For example, the present systems can be used for quantitatively detecting 2 markers, such as: insulin-like growth factor-binding protein 7 and metallopeptidase inhibitor 2; insulin-like growth factor-binding protein 7 and neutrophil elastase; insulin-like growth factor-binding protein 7 and hyaluronic acid; metallopeptidase inhibitor 2 and neutrophil elastase; metallopeptidase inhibitor 2 and hyaluronic acid; neutrophil elastase and hyaluronic acid. The present systems can be used for quantitatively detecting 3 markers, such as: insulin-like growth factor-binding protein 7, metallopeptidase inhibitor 2 and neutrophil elastase; insulin-like growth factor-binding protein 7, metallopeptidase inhibitor 2, and hyaluronic acid; insulin-like growth factor-binding protein 7, neutrophil elastase, and hyaluronic acid; metallopeptidase inhibitor 2, neutrophil elastase and hyaluronic acid. The present systems can be used for quantitatively detecting all 4 markers: insulin-like growth factor-binding protein 7, metallopeptidase inhibitor 2, neutrophil elastase, and hyaluronic acid.

### E. Exemplary Embodiments

An exemplary test system, e.g., the Astute NEPHROCHECK™ Test, employs a sandwich immunoassay technique along with lateral flow membrane and fluorescence detection technology to quantitatively measure up to two to four protein biomarkers in human samples, e.g., human urine samples, quickly, e.g., in approximately twenty minutes. In some embodiments, the sample is about 100 μL fresh or thawed (e.g., previously frozen) human urine sample.

Briefly, the test procedure involves mixing adult, human urine samples (100 μL fresh or thawed—previously frozen) with fluorescent antibody conjugate reagent. The fluorescent antibody conjugate reacts with the biomarkers present in the urine specimen. The urine and fluorescent antibody-conjugated specimen mixture is then added to the sample port on the Test cartridge and the Test cartridge is inserted into the ASTUTE140 Meter. The urine and fluorescent antibody-conjugated specimen migrates across the Test cartridge by capillary action. The presence of the protein biomarkers in the specimen causes formation of the fluorescent antibody conjugate/biomarker/capture antibody sandwiches in detection zones on Test cartridge membrane. Approximately twenty minutes after the Test cartridge is inserted into the Meter, the Meter determines the concentration of each of the biomarkers, multiplies the concentrations for each of the biomarkers into a single numerical test result, and displays the result to the user on the Meter screen. The Test result can be printed via a thermal printer internal to the Meter. If connected (e.g., by LAN or USB), the Meter can transmit results to a laboratory information system (LIS).

NEPHROCHECK Test Cartridge Kit

The NEPHROCHECK Test Kit comprises the following components:
NEPHROCHECK Test;
NEPHROCHECK Test Conjugate Vial;
NEPHROCHECK Test RFID (Radio-frequency Identification) Card;
NEPHROCHECK Test Buffer Solution; and
Package Insert.

NEPHROCHECK Test Cartridge

The NEPHROCHECK Test cartridge is a single-use cartridge comprising a membrane test strip enclosed in a plastic housing. The cartridge housing is customized and designed to uniquely fit into the drawer of the ASTUTE140 Meter thus serving as a “closed system”. The test strip is comprised of a nitrocellulose membrane, wick pad and sample pad laminated to a backing card and mounted on the bottom cartridge housing. The top plastic housing contains two openings; one rectangular opening (otherwise known as a ‘test’ window) and one round opening (otherwise known as the sample port). The rectangular opening outlines the area of the membrane test strip where the capture antibodies and internal controls have been deposited during the manufacturing process. The test strip has the capability to have up to five zones (three detection and two control zones). The current design comprises 4 zones (two biomarker detection zones and two control zones). Antibodies that bind to the biomarkers are pre-deposited onto discrete assay detection zones (one detection zone for each biomarker) on the nitrocellulose membrane. An additional two zones are used for pre-deposited internal control (one zone for positive control and one zone for negative control).

The round port is utilized for sample application. A specified amount of urine is mixed with fluorescent antibody conjugate reagent and then added to the port to begin the reaction.

The top housing of the NEPHROCHECK Test cartridge has a printed barcode containing the cartridge lot ID and cartridge serial number. When inserted into the ASTUTE140 Meter and the Meter drawer is closed, a barcode reader internal to the Meter reads the barcode on the Test cartridge confirming the RFID card for the cartridge lot has been read by the Meter.

NEPHROCHECK Conjugate Vial and NEPHROCHECK Test Buffer Solution

Each NEPHROCHECK Test is provided with a single use vial of soluble fluorescent antibody conjugate reagent supplied as a lyophilized solid. NEPHROCHECK Test Buffer Solution is provided with the kit to reconstitute the fluorescent antibody conjugate reagent. This reagent contains multiple fluorescently-labeled antibodies that bind to the two protein biomarkers. When the operator is ready to run the test, the lyophilized conjugate is reconstituted by adding a specified amount of buffer. A specified amount of urine is then deposited into the vial containing the reconstituted conjugate. The operator then deposits a specified amount of the urine/conjugate mixture and places into the sample well on the Test cartridge.

RFID Cards

Lot specific radio-frequency identification (RFID) cards will be supplied with each NEPHROCHECK Test Kit. Each RFID card is embedded with an antenna and an RFID tag. The NEPHROCHECK Test Kit RFID card contains lot specific information which includes the lot ID, expiration date, and assay calibration parameters. These calibration parameters determine calibration curves for each of the two biomarker specific detection zones. Each curve represents the fluorescence signal measured for each biomarker detection zone with a known biomarker. Prior to running a new lot of Test cartridges in the Meter, the NEPHROCHECK Test lot specific RFID card must be read by the Meter. If a NEPHROCHECK Test cartridge is inserted into a Meter to which the RFID card has not been read, when the Meter reads the barcode on the cartridge it will recognize the RFID card for the lot has not been read by the Meter and the test will not run.

Package Insert

The NEPHROCHECK Test Kit Package Inserts provide indications for use and specific technical information related to performance.

ASTUTE140 Meter Kit

The ASTUTE140 Meter Kit contains the following components:
ASTUTE140 Meter;
ASTUTE140 Meter User Manual; and
Universal Power Supply.

ASTUTE140 Meter

The ASTUTE140 Meter is a bench-top/table-top reader that utilizes a fluorescence optical system to quantitatively determine the amount of analyte present on the test cartridge. The drawer has been custom designed to hold a single NEPHROCHECK Test cartridge as a “closed system”. The bottom of the test cartridge has specific design components which allow it to be inserted into the drawer in only one orientation.

Upon inoculation of a test cartridge with fluorescent antibody-conjugated specimen the Test cartridge is inserted into the ASTUTE140 Meter, and an LED (Light-emitting diode) illuminates the Test cartridge. The Meter utilizes a fluorescence optical system to measure the fluorescence signal across each of the NEPHROCHECK Test cartridge's 4 detection zones; 2 biomarker detection zones and 2 control zones. The fluorescent signal from each of the 2 protein biomarker detection zones corresponds to the concentration of biomarkers present in the sample. The Meter also detects the fluorescent signals from the 2 control zones. If the automatic check of these “built-in controls” shows that the resulting control values are within the limits set during manufacturing, the ASTUTE140 Meter converts the fluorescence signal for each of the 2 protein biomarker detection zones into a concentration using the lot-specific calibration information stored in the NEPHROCHECK Test RFID Card provided with the test kit. The Meter then multiplies the concentrations for each of the protein biomarkers on the NEPHROCHECK Test into a single numerical test result and displays this result to the user. The results from the individual biomarkers are not displayed—only the single numerical test result is displayed.

The ASTUTE140 Meter is operated via a LCD (Liquid crystal display) color graphic display with backlighting and meter keypad (2 soft keys, 3 functional keys (eject, print, paper feed), 4 arrow keys (up, down, left, right) and 12 numeric keys. A virtual keypad may be used to enter characters; alternatively, an external keypad may be attached for convenience. The ASTUTE140 Meter is operated with on-board controllers that communicate with the graphical User Interface and Analysis Module. The on-board controllers schedule, manage, drive all motors actuators, sensors, etc. in order to execute tests and provide results.

The ASTUTE140 Meter is equipped with a RFID reader and barcode reader. The RFID reader is used to transfer lot-specific information from the RFID cards to the non-volatile memory in the Meter. The internal barcode reader is used to read the barcodes printed on the NEPRHOCHECK Test cartridges.

The ASTUTE140 Meters will be factory calibrated by adjusting the optical output using physical standards that fit in the cartridge holder. The Meter will be designed to contain a close-looped feedback system to stabilize the optical illumination for reading the Test device.

User Manual

The set-up, use, and care of the ASTUTE140 Meter are described in the User Manual provided with the purchase of the Meter. The ASTUTE140 Meter does not require servicing (e.g., preventive maintenance care).

Intended Use

The NEPHROCHECK™ Test is an in vitro diagnostic device that quantitatively measures TIMP-2 (Tissue Inhibitor of Metalloproteinase 2) and IGFBP-7 (Insulin-like Growth Factor Binding Protein 7) proteins associated with kidney function in human urine by fluorescence immunoassay on the ASTUTE140™ Meter. The test result is intended to be used in conjunction with clinical evaluation as an aid in the risk assessment of acute kidney injury in the critically ill. The NEPHROCHECK™ Test is indicated for prescription use only.

### Summary and Explanation

Acute kidney injury (AKI) is one of the more prevalent and serious morbidities in critically ill hospitalized patients and is associated with a multitude of acute and chronic conditions.¹⁻⁶ The economic and public health burden of AKI is staggering with substantially increased mortality, morbidity, length of ICU stay and in-hospital costs, as well as longer term health consequences.⁷⁻¹³ Tests to assess AKI provide important information to physicians and, in conjunction with other available clinical information, can aid physicians in optimizing subject management.^{4,13-15}

Principles of the NEPHROCHECK™ Test Procedure

The Astute Medical NEPHROCHECK™ Test and ASTUTE140™ Meter employ a sandwich immunoassay technique along with fluorescence detection technology to quantitatively measure protein biomarkers in fresh or thawed (e.g., previously frozen) human urine samples in approximately twenty minutes.

The NEPHROCHECK™ Test is a single-use cartridge designed to be uniquely compatible with the ASTUTE140™ Meter. When the ASTUTE140™ Meter is used in conjunction with the NEPHROCHECK™ Test, the ASTUTE140™ Meter converts the fluorescent signals for the individual immunoassays into TIMP-2 and IGFBP-7 concentrations and combines these individual concentrations into a single numerical test result.

Materials Provided

The NEPHROCHECK™ Test cartridge and NEPHROCHECK™ Test Kit contain all the reagents needed for the generation of NEPHROCHECK™ Test results in human adult urine specimens. The NEPHROCHECK™ Test cartridge and NEPHROCHECK™ Test Conjugate Vial contain:
Murine monoclonal and goat polyclonal antibodies against TIMP-2;
Murine monoclonal and goat polyclonal antibodies against IGFBP-7;
Fluorescent dye;
Stabilizers; and
Excipients.

The NEPHROCHECK™ Test Kit containing:
NEPHROCHECK™ Test . . . 25;
NEPHROCHECK™ Test Conjugate Vial [symbol] . . . 25;
NEPHROCHECK™ Test RFID Card [symbol] . . . 1;
NEPHROCHECK™ Test Buffer (2×5 mL) [symbol] . . . 1; and
NEPHROCHECK™ Test Kit Package Insert . . . 1.

Materials Not Provided

Materials required but not provided:
ASTUTE140™ Meter (PN 500000);
NEPHROCHECK™ Liquid Control Kit (PN 500005);
NEPHROCHECK™ Electronic Quality Control (PN 400013); and
Calibrated precision pipette, capable of dispensing 100 μL.

Warnings and Precautions

Warnings and precautions include the following:
For in vitro diagnostic use.
The NEPHROCHECK™ Test is intended for use by trained medical professionals.
Do not use the NEPHROCHECK™ Test Kit beyond the expiration date printed on the outside of the box.
Carefully follow the instructions and procedures described in this insert.
Keep the NEPHROCHECK™ Test cartridge and NEPHROCHECK™ Conjugate Vial in the sealed pouch until ready for immediate use.
Patient specimens, used NEPHROCHECK™ Test cartridges and used pipette tips may be potentially infectious. Proper handling and disposal methods in compliance with federal and local regulations should be established.
The NEPHROCHECK™ Test is to be used only with the ASTUTE140™ Meter and the NEPHROCHECK™ Liquid Control Kit.
The NEPHROCHECK™ Test Conjugate Vials contained in the NEPHROCHECK™ Test Kit are to be used only with the NEPHROCHECK™ Test cartridges contained in the same kit box. The NEPHROCHECK™ Test Conjugate Vials are not to be used with cartridges that are contained in other boxes or provided with other products.
The NEPHROCHECK™ Test Kit requires the use of calibrated precision pipette(s). It is recommended that users review the proper procedures for the use of these devices in order to ensure accurate dispensing of volumes.
In order to minimize contamination, pipette tips are to be discarded and a new one used for each new specimen.

Storage and Handling Requirements

Storage and handling requirements include the following:
Prior to using the NEPHROCHECK™ Test Kit, inspect the kit components for damage. Do not use the NEPHROCHECK™ Test Kit if you encounter damage.
The NEPHROCHECK™ Test Conjugate Vial material is lyophilized.
The unopened NEPHROCHECK™ Test Kit components are stable until the expiration date printed on the box when stored at 4-25° C. (39.2-77° F.).
The opened NEPHROCHECK™ Test Buffer is stable to the expiration date printed on the bottle label or until 28 days after initial opening of the bottle (whichever occurs first) when the unused portion is properly stored at 4-25° C. (39.2-77° F.).
Each NEPHROCHECK™ Test and NEPHROCHECK™ Test Conjugate Vial is intended for single use only.
After completion of all tests included in the kit box, dispose of any remaining NEPHROCHECK™ Test Buffer in accordance with local regulations.
If kit materials are stored refrigerated, allow the kit components to reach operating temperature of 18-25° C. (64-77° F.).

ASTUTE140™ Meter Configuration

Before running the NEPHROCHECK™ Test, the ASTUTE140™ Meter must be configured and NEPHROCHECK™ Liquid Quality Control (LQC) and NEPHROCHECK™ Electronic Quality Control (EQC) procedures “passed” (See “Installation” and “ASTUTE140™ Meter Operation” in the ASTUTE140™ Meter User Manual for detailed instructions).
1. If necessary, register the ASTUTE140™ EQC device using the ASTUTE140™ Electronic Quality Control (EQC) RFID card.
2. If necessary, run the ASTUTE140™ Electronic Quality Control procedure.
3. Register and run NEPHROCHECK™ Liquid Control Kit as needed.

NEPHROCHECK™ Test Preparation

Before running the NEPHROCHECK™ Test, the following must be completed: Register a NEPHROCHECK™ Test lot using the NEPHROCHECK™ RFID Card enclosed in the NEPHROCHECK™ Test Kit. If registered correctly, a screen indicating that the lot number and expiration date was successfully read from the NEPHROCHECK™ RFID Card will appear and the lot number and expiration date will be displayed (See “Test Lot Registration” in the ASTUTE140™ User Manual for detailed instructions).

### Specimen Collection and Preparation

The NEPHROCHECK™ Test is intended for use with fresh or frozen adult human urine specimens only. Other specimen types have not been characterized. The following steps are used for the non-frozen samples:
1. Collect a fresh urine sample of approximately 10 mL in a clean specimen collection cup without additives. For patients with indwelling bladder catheters, the collection bag should first be emptied and then a fresh sample of urine should be collected; alternatively, the sample may be collected from an urometer if present. Transport the urine sample to the laboratory that will run the NEPHROCHECK™ Test.
2. Samples should be transferred to the laboratory and centrifuged within two hours of sample collection. If the sample cannot be tested within two hours, the sample may be refrigerated up to 24 hours or flash frozen and stored at ≦−70° C. (−94° F.) until it can be tested. Avoid repeated freezing and thawing of samples.
3. Transfer urine sample from specimen collection cup to a clean centrifuge tube. Centrifuge the urine sample for 10 minutes at 1000×g at 4° C. (39.2° F.). Transfer supernatant to a clean receptacle. Allow supernatant to reach room temperature.
4. Test centrifuged sample within four hours of sample collection.

The following steps are used for the frozen samples:
1. To test frozen samples, thaw urine samples in a room temperature (18-23° C.; 64.4-73.4° F.) water bath for 15 minutes.
2. Once the sample is thawed, gently invert the sample tube 1-2 times to mix sample.
3. Frozen samples must be inoculated into a NEPHROCHECK™ Test cartridge within one hour of placing the patient sample into the water bath.

### NEPHROCHECK™ Test Procedure

The Test procedure requires the use of a calibrated precision pipette for the following: addition of NEPHROCHECK™ Test Buffer Solution and urine sample into the NEPHROCHECK™ Test Conjugate Vial and introduction of sample into the NEPHROCHECK™ Test cartridge. Prior to running the test, the NEPHROCHECK™ Test cartridge lot must be registered (See “Test Lot Registration” in the ASTUTE140™ Meter User Manual) and NEPHROCHECK™ Test Kit components must be at the operating temperature of 18-25° C. (64-77° F.). To perform the NEPHROCHECK™ Test, follow these steps:

1. Preparation:
a. Highlight and select Run Patient on the ASTUTE140™ Meter Main Menu.
b. Manually enter the Patient ID or scan the Patient ID into the ASTUTE140™ Meter using a barcode scanner (if connected). After confirming that the correct Patient ID and/or Sample ID have been entered, select Run Patient. The ASTUTE140™ Meter drawer will automatically open.
c. Remove the new NEPHROCHECK™ Test cartridge from the foil pouch and place on a flat surface.
d. Remove the NEPHROCHECK™ Test Conjugate Vial from the pouch.
e. Remove the cap from the NEPHROCHECK™ Test Conjugate Vial. Visually inspect to ensure that no bead has adhered to the cap. If any bead has adhered, place the cap on vial and tap three times. Repeat until there is no bead inside the cap.
f. Pipette 100 μL of NEPHROCHECK™ Test Buffer Solution into the NEPHROCHECK™ Test Conjugate Vial. Discard the pipette tip in accordance with local regulations. The conjugate liquid in the vial is to be used as soon as it is reconstituted.
g. Using a new pipette tip, add 100 μL of centrifuged urine or liquid control sample to the NEPHROCHECK™ Test Conjugate Vial. Mix thoroughly (mix at least three times using the pipette tip).
h. Pipette 100 μL of sample/conjugate solution onto the designated sample port on the NEPHROCHECK™ Test cartridge. Wait approximately one minute for the sample to be absorbed into the round well.

2. Run the NEPHROCHECK™ Test:
a. Using the grips on the side of the NEPHROCHECK™ Test cartridge, position the cartridge inside the ASTUTE140™ Meter drawer with the Astute Medical logo towards the inside of the meter drawer. Keep the NEPHROCHECK™ Test cartridge horizontal and avoid tipping the test cartridge during placement into the ASTUTE140™ Meter drawer.
b. Close the ASTUTE140™ Meter drawer. In approximately 20 minutes, a single numerical test result will be displayed.
c. Eject the ASTUTE140™ Meter drawer. Remove the NEPHROCHECK™ Test cartridge and discard it and the conjugate vial in accordance with local regulations.

3. Review the NEPHROCHECK™ Test Results:
Upon completion of running the test, follow instructions in the ASTUTE140™ Meter User Manual to print results (if desired) or upload results to the Laboratory Information System (LIS).
If the NEPHROCHECK™ Test should fail, a meter error message will indicate that the result is invalid and that a new cartridge should be run. If the procedure fails a second time, contact Astute Technical Support.

NEPHROCHECK™ Test Preparation Process

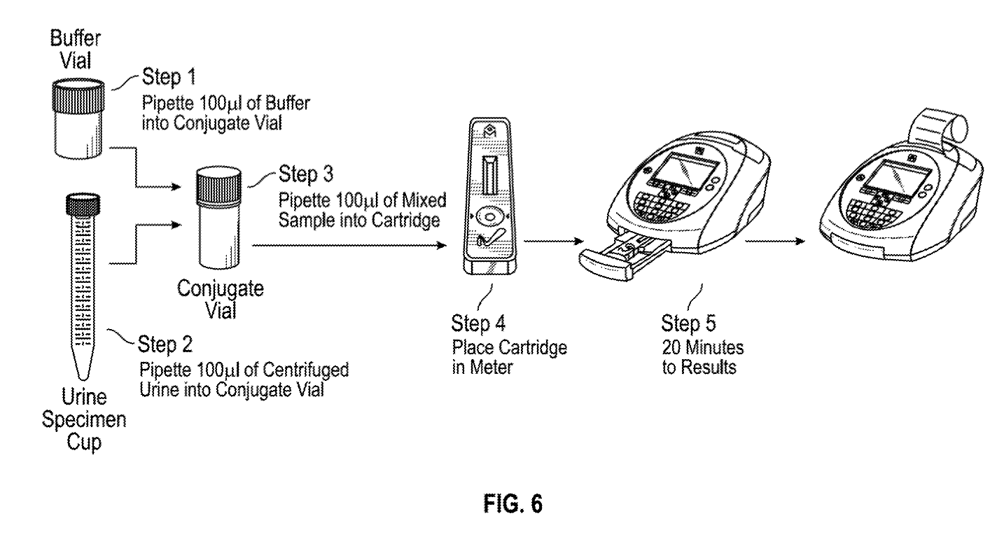

NEPHROCHECK™ Test preparation process is illustrated in FIG. 6.

NEPHROCHECK™ RFID Card

The NEPHROCHECK™ Test RFID Card contains information such as the lot number and the expiration date of the NEPHROCHECK™ Test cartridges. This information is transferred from the NEPHROCHECK™ Test RFID Card to the ASTUTE140™ Meter during registration of the NEPHROCHECK™ Test Kit. Lot number and expiration date can be accessed through the ASTUTE140™ Meter at any time (See “Test Lot Registration” in the ASTUTE140™ Meter User Manual).

### Results

The ASTUTE140™ Meter automatically calculates the NEPHROCHECK™ Test result as a single numerical risk result that is displayed on the ASTUTE140™ Meter screen after the NEPHROCHECK™ Test procedure is completed; results for the individual markers are not displayed. The NEPHROCHECK™ Test result is determined as follows: ([IGFBP-7]*[TIMP-2])/1000. The test result is displayed without units. The NEPHROCHECK™ Test results are also stored in the ASTUTE140™ Meter memory and may be accessed at any time (See “Review and Management of Test Results” in the ASTUTE140™ Meter User Manual).

### Standardization

Concentration results for each of the assays contained in the NEPHROCHECK™ Test are traceable to reference standard solutions that contain defined mass (concentration) of TIMP-2 and IGFBP-7 proteins in accordance with EN ISO 17511. The NEPHROCHECK™ Test and NEPHROCHECK™ Liquid Controls are traceable to the same reference standard solutions.

### Quality Control Considerations

Each NEPHROCHECK™ Test cartridge contains two detection zones used as internal controls (one positive and one negative control). These positive and negative controls are run automatically with every sample, in order to confirm the integrity of the NEPHROCHECK™ Test cartridge and the performance of the ASTUTE140™ Meter. If the automatic check of these internal controls shows that the control value results are not within pre-defined limits, the Meter will display an error message and the Test result will not be reported. These controls are in addition to the external NEPHROCHECK™ Liquid Controls. Good Laboratory Practice suggests that external NEPHROCHECK™ Liquid Controls be tested:
Every 30 days;
With each new lot number of NEPHROCHECK™ Test Kits;
With each new shipment of the NEPHROCHECK™ Test Kits; and
In accordance with your laboratory standard quality control procedures.
   Performing System Quality Control with the ASTUTE140™ Electronic Quality Control Device (EQC)

The EQC procedure verifies the calibration of the ASTUTE140™ Meter to confirm that the ASTUTE140™ Meter is functioning properly. Perform EQC testing:
Upon initial set up of the ASTUTE140™ Meter;
In accordance with your laboratory standard quality control procedures;
Prior to running the first EQC procedure, the ASTUTE140™ EQC Device must be registered (See “ASTUTE140™ EQC Device Registration” in the ASTUTE140™ Meter User Manual).
If the procedure fails, repeat the procedure (See “ASTUTE140™ EQC Device Registration” in the ASTUTE140™ Meter User Manual).

When not in use, the ASTUTE140™ EQC Device should be stored in the case provided away from direct light as indicated on the product label. Do not discard the ASTUTE140™ EQC Device. If lost or damaged, a replacement ASTUTE140™ EQC Device may be ordered by contacting your closest Astute Medical, Inc. sales representative or the Astute Medical Inc. Technical Services department.

### Limitations of the NEPHROCHECK™ Test Procedure

Test results should be evaluated in the context of all clinical and laboratory data available. In those instances where the test results do not agree with the clinical evaluation, additional tests should be performed accordingly.

### Performance Characteristics

### Analytical Sensitivity

The limit-of-blank (LoB) was determined for each of biomarker assays contained within the NEPHROCHECK™ Test in accordance with the methods provided in CLSI guideline EP17-A¹⁷. A blank urine sample was evaluated on a total of 240 tests from three different lots of test kits (80 tests per lot). These data were collected over 40 separate runs that were conducted twice a day over 20 total days of testing. The limit-of-blank is the 95th percentile of the measured results. The limit-of-blank of each assay is presented below in Table 2:

TABLE 2

| Biomarker | Limit-of-Blank |
|---|---|
| TIMP-2 | 0.6 ng/ml |
| IGFBP-7 | 0.7 ng/ml |

In addition, the limit-of-detection (LoD) and limit-of-quantitation (LoQ) were also determined for each of the biomarker assays. Six human urine samples that contained low levels of both biomarkers were tested with 60 tests from three lots of test kits (20 tests per lot). These data were collected over 10 separate runs that were conducted twice a day over 5 total days of testing. The measured results were analyzed as described in CLSI guideline EP17-A¹⁷. Representative results of this analysis are presented below in Table 3:

TABLE 3

| | Limit-of- | Limit-of- |
|---|---|---|
| Biomarker | Detection | Quantitation |
| TIMP-2 | 1.1 ng/ml | 1.1 ng/ml |
| IGFBP-7 | 3.6 ng/ml | 3.6 ng/ml |

Linearity

The linearity of the biomarker assays contained in the NEPHROCHECK™ Test were evaluated in accordance with CLSI guideline EP6-A¹⁶. Three urine samples that contained various levels of TIMP-2 and IGFBP-7 were mixed with 3 separate urine samples that contained low levels of TIMP-2 and IGFBP-7. These samples were mixed to prepare 11 test samples with TIMP-2 concentrations from 0.8 ng/ml to 250 ng/ml and 10 test samples with IGFBP-7 concentrations from 26 ng/ml to 620 ng/ml. All samples were tested with at least 9 tests from a single lot of test kits. The concentration results for both TIMP-2 and IGFBP-7 were within 15 percent of their expected values for all test samples. The measurable ranges are shown in the following Table 4.

TABLE 4
Measureable Ranges

| TIMP-2: | 1.2-225 ng/ml |
|---|---|
| IGFBP-7: | 20-600 ng/ml |
| NephroCheck Test Result: | 0.02-135 |

Precision

The reproducibility of the biomarker assays contained in the NEPHROCHECK™ Test was determined by testing multiple, human urine based control samples with three different lots of NEPHROCHECK™ Tests. Testing was completed in accordance with the methods described in CLSI guideline EP5-A2¹⁸. Each control sample was evaluated on a total of at least 240 tests from three different lots of test kits (80 tests per lot). These data were collected over 40 separate runs that were conducted twice a day over at least 20 total days of testing. Study results were analyzed as described in CLSI guideline EP5-A2¹⁸. Representative results of this analysis are presented below in Table 5.

TABLE 5

| | | Mean | Within-Run | | Total | |
|---|---|---|---|---|---|---|
| | Control | Concentration | Precision | | Precision | |
| Biomarker | Sample | (ng/ml) | SD | % CV | SD | % CV |
| TIMP-2 | Control 1 | 2.7 | 0.3 | 10.7% | 0.3 | 11.4% |
| | Control 2 | 139 | 11.1 | 8.0% | 11.3 | 8.1% |
| IGFBP-7 | Control 1 | 37.1 | 2.9 | 7.7% | 2.9 | 7.9% |
| | Control 2 | 211 | 13.2 | 6.3% | 14.0 | 6.6% |

Interfering Substances

The following substances were evaluated for interference with the biomarker assays contained in the NEPHROCHECK™ Test. These substances were evaluated in accordance with the methods described in CLSI guideline EP7-A2¹⁹. Each substance was added to a human urine pool that contained approximately 3 ng/ml TIMP-2 and 50 ng/ml IGFBP-7. None of the substances impacted TIMP-2 or IGFBP-7 assay results at the concentrations listed Table 6 below. While no interference was observed at the concentrations tested, interference may exist at higher concentrations.

TABLE 6

| Substance | Test Concentration | |
|---|---|---|
| Acetone | 12,000 | umol/L |
| Albumin | 60 | mg/ml |
| Ascorbic Acid | 170 | umol/L |
| Sodium Bicarbonate | 35,000 | umol/L |
| Bilirubin, Conjugated | 340 | umol/L |
| Bilirubin, Unconjugated | 270 | umol/L |
| Creatinine | 440 | umol/L |
| Ethanol | 22,000 | umol/L |
| Glucose | 55,000 | umol/L |
| Hemoglobin | 2,000 | ng/ml |
| Riboflavin | 10,600 | umol/L |
| Urea | 430,000 | umol/L |

Interfering Conditions

The effect of urine sample pH was evaluated for each of the biomarker assays contained on the NEPHROCHECK™ Test. Two human urine pools were adjusted to multiple pH values between pH 4 and 10. One urine pool contained approximately 3 ng/ml TIMP-2 and 60 ng/ml IGFBP-7. The other urine pool contained approximately 125 ng/ml TIMP-2 and 250 ng/ml IGFBP-7. For both urine pools, urine sample pH did not impact TIMP-2 or IGFBP-7 assay results.

Pharmaceuticals

The following pharmaceuticals were evaluated for interference with the biomarker assays contained in the NEPHROCHECK™ Test. These pharmaceuticals were evaluated in accordance with the methods described in CLSI guideline EP7-A2¹⁹. Each pharmaceutical was added to a human urine pool containing approximately 3 ng/ml TIMP-2 and 50 ng/ml IGFBP-7. Each drug was tested at a concentration at least equivalent to the maximum therapeutic level. None of the pharmaceuticals listed in Table 7 below impacted TIMP-2 or IGFBP-7 results.

TABLE 7

Acetaminophen
Aspirin
Caffeine
Ciprofloxacin
Dopamine
Fentanyl
Furosemide
Heparin
Hydrocodone
Ibuprofen
Insulin
Levofloxacin
Lisinopril
Methylene Blue
Metoprolol
Midazolam
Morphine
Ondansetron
Penicillin
Propofol
Vancomycin

Proteins

The biomarker assays contained in the NEPHROCHECK™ Test were evaluated for cross-reactivity with the related proteins listed in the Table 8 below. Each protein was added to a human urine pool containing approximately 3 ng/ml TIMP-2 and 50 ng/ml IGFBP-7. Each sample was tested with 25 or more NEPHROCHECK™ Tests. The testing results are shown in Table 8 below.

TABLE 8
Cross-Reactivity with Related Protein

| | | TIMP-2 | IGFBP-7 |
|---|---|---|---|
| Protein | ng/mL | % Cross-reactivity | % Cross-reactivity |
| IGF-1 | 375,000 | — | 0 |
| IGF-2 | 375,000 | — | 0 |
| IGFBP-1 | 200,000 | — | 0 |
| IGFBP-2 | 2,000 | — | 0 |
| TIMP-1 | 2,500,000 | 0 | — |
| TIMP-3 | 2,500,000 | 0 | — |
| TIMP-4 | 2,500,000 | 0 | — |

### Clinical Performance

### Critically Ill Study Cohort

Urine samples collected from critically ill adult subjects were used to validate the NEPHROCHECK™ Test as an aid in the risk assessment for AKI in the critically ill. These samples were collected as part of a multi-center, prospective study conducted at 35 clinical sites across North America and Europe. The study targeted subjects within 24 hours of ICU admission who did not have known moderate or severe AKI (RIFLE-I or RIFLE-F; AKIN 2 or AKIN 3) at enrollment, were expected to be in the ICU (any type of ICU) for at least 48 hours with a urinary catheter in place as standard care, and who had hemodynamic and/or respiratory dysfunction. Each subject in the study cohort had up to three urine biomarker samples collected within 18 hours after the time of enrollment. The study cohort comprised 629 subjects; 60.8% were male, 78.5% were white/Caucasian, and the mean (±SD) age was 62 (±16) years.

Acute kidney injury status was determined using the full RIFLE criteria (based on serum creatinine and urine output values). (See e.g., Bellomo, R., Ronco, C., Kellum, J. A., Mehta, R. L., and Palevsky, P. (2004) Acute renal failure—definition, outcome measures, animal models, fluid therapy and information technology needs: the Second International Consensus Conference of the Acute Dialysis Quality Initiative (ADQI) Group, Crit Care 8, R204-R212) An observation of RIFLE-I or RIFLE-F within the 12 hour interval starting from the time of each sample collection to 12 hours after the collection was classified as positive for moderate or severe AKI while absence of RIFLE-I or RIFLE-F within the 12 hour interval was classified as negative for moderate or severe AKI for the sample. Of the 629 subjects in the study cohort, 79 were classified as positive for moderate or severe AKI for at least one sample collection.

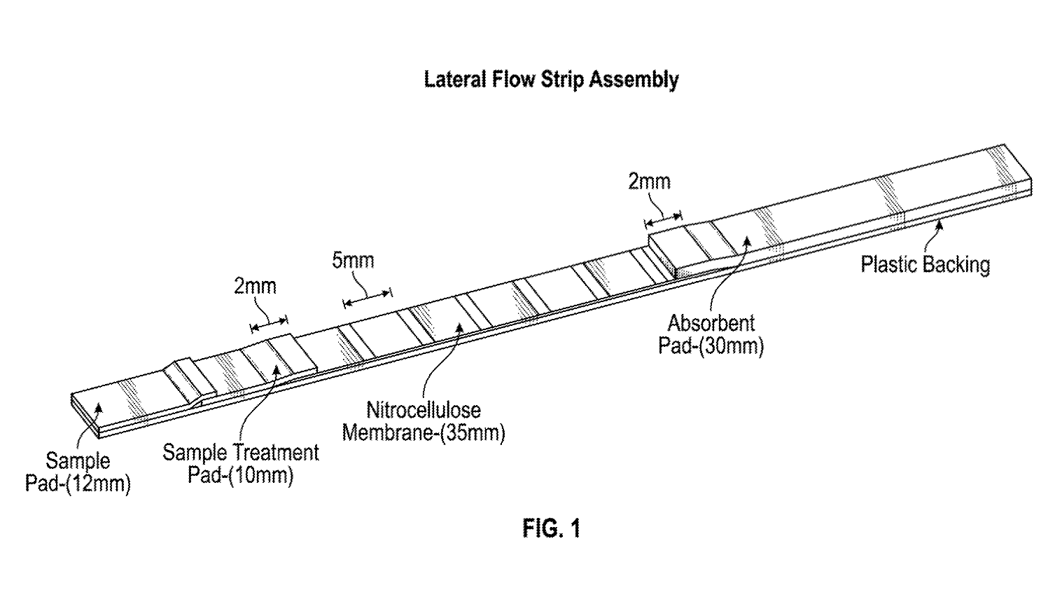

NEPHROCHECK Test values for study cohort samples were divided into tertiles defined by the 33^{rd} and 67^{th} percentiles of values obtained for the entire study cohort. The 33^{rd} and 67^{th} percentiles corresponded to NEPHROCHECK Test values of 0.16 and 0.52, respectively. The risk (corresponding to probability) of moderate or severe AKI was calculated for each tertile and was found to increase monotonically (p<0.0001) with increasing tertile as follows: for tertile 1, risk=2.0%; for tertile 2, risk=5.9%; for tertile 3, risk=21%. The relative risk (95% CI) of AKI was 2.9 (1.5-7.1) and 10.3 (6.1-24.8) for the second compared to the first tertile and the third compared to the first tertile, respectively (FIG. 1).

### Apparently Healthy Cohort

NEPHROCHECK Test results for urine samples collected from 383 apparently healthy adult subjects were used to establish the reference range for healthy subjects. Of this cohort, 45.6% were male and 68.1% were white/Caucasian. The mean (±SD) age was 57 (±16) years. Reference ranges were determined using the nonparametric method. The reference range corresponding to the 2.5^{th} to 97.5^{th} percentile was 0.02 to 1.93 for healthy subjects (Table 9 below). NEPHROCHECK Test values at other commonly reported percentiles are provided in Table 9. For comparison, Table 9 also provides results for samples collected from the subjects in the critically ill study cohort, grouped by maximum RIFLE stage within 12 hours of sample collection. These reference ranges are provided as guidelines only and are not intended to be critical values or medical decision limits. Each laboratory should establish its own reference intervals. Guidance for establishing reference intervals can be found in CLSI Guideline C28-A3c.

TABLE 9
NEPHROCHECK Test values at specified percentiles determined for
samples collected from Healthy Subjects and Critically Ill Subjects.
Samples from Critically Ill Subjects were grouped by maximum
RIFLE stage within 12 hours of sample collection.

| NephroCheck Test Values | |
|---|---|
| Healthy | Critically Ill Subjets |

| Percentile | Subjects | No AKI | RIFLE R | RIFLE I or F |
|---|---|---|---|---|
| 2.5 | 0.02 | 0.02 | 0.03 | 0.10 |
| 5 | 0.03 | 0.03 | 0.04 | 0.15 |
| 10 | 0.03 | 0.04 | 0.06 | 0.21 |
| 25 | 0.07 | 0.09 | 0.16 | 0.49 |
| 50 | 0.22 | 0.23 | 0.43 | 1.22 |
| 75 | 0.58 | 0.53 | 0.96 | 2.97 |
| 90 | 1.00 | 1.10 | 2.12 | 6.16 |
| 95 | 1.34 | 1.66 | 3.12 | 7.71 |
| 97.5 | 1.93 | 2.22 | 5.95 | 9.38 |

### LITERATURE REFERENCES

1. Uchino, S., Kellum, J. A., Bellomo, R., Doig, G. S., Morimatsu, H., Morgera, S., Schetz, M., Tan, I., Bouman, C., Macedo, E., Gibney, N., Tolwani, A., and Ronco, C. Acute renal failure in critically ill patients: a multinational, multicenter study. JAMA 294, 813-818 (2005).
2. Mehta, R. L., Pascual, M. T., Soroko, S., Savage, B. R., Himmelfarb, J., Ikizler, T. A., Paganini, E. P., and Chertow, G. M. Spectrum of acute renal failure in the intensive care unit: the PICARD experience. Kidney Int. 66, 1613-1621 (2004).
3. Waikar, S. S., Liu, K. D., and Chertow, G. M. Diagnosis, epidemiology and outcomes of acute kidney injury. Clin. J. Am. Soc. Nephrol. 3, 844-861 (2008).
4. Waikar, S. S., Liu, K. D., and Chertow, G. M. Diagnosis, epidemiology and outcomes of acute kidney injury. Clin. J. Am. Soc. Nephrol. 3, 844-861 (2008).
5. Kellum, J. A. Acute kidney injury. Crit Care Med. 36, 5141-5145 (2008).
6. Xue, J. L., Daniels, F., Star, R. A., Kimmel, P. L., Eggers, P. W., Molitoris, B. A., Himmelfarb, J., and Collins, A. J. Incidence and mortality of acute renal failure in Medicare beneficiaries, 1992 to 2001. J. Am. Soc. Nephrol. 17, 1135-1142 (2006).
7. McCullough, P. A., Adam, A., Becker, C. R., Davidson, C., Lameire, N., Stacul, F., and Tumlin, J. Epidemiology and prognostic implications of contrast-induced nephropathy. Am. J. Cardiol. 98, 5K-13K (2006).
8. Joannidis, M., Metnitz, B., Bauer, P., Schusterschitz, N., Moreno, R., Druml, W., and Metnitz, P. G. Acute kidney injury in critically ill patients classified by AKIN versus RIFLE using the SAPS 3 database. Intensive Care Med. 35, 1692-1702 (2009).
9. Dasta, J. F., Kane-Gill, S. L., Durtschi, A. J., Pathak, D. S., and Kellum, J. A. Costs and outcomes of acute kidney injury (AKI) following cardiac surgery. Nephrol. Dial. Transplant. 23, 1970-1974 (2008).
10. Bagshaw, S. M., George, C., Dinu, I., and Bellomo, R. A multi-centre evaluation of the RIFLE criteria for early acute kidney injury in critically ill patients. Nephrol. Dial. Transplant. 23, 1203-1210 (2008).
11. Hoste, E. A., Clermont, G., Kersten, A., Venkataraman, R., Angus, D. C., De, B. D., and Kellum, J. A. RIFLE criteria for acute kidney injury are associated with hospital mortality in critically ill patients: a cohort analysis. Crit Care 10, R73 (2006).
12. Chertow, G. M., Burdick, E., Honour, M., Bonventre, J. V., and Bates, D. W. Acute kidney injury, mortality, length of stay, and costs in hospitalized patients. J. Am. Soc. Nephrol. 16, 3365-3370 (2005).
13. Amdur, R. L., Chawla, L. S., Amodeo, S., Kimmel, P. L., and Palant, C. E. Outcomes following diagnosis of acute renal failure in U.S. veterans: focus on acute tubular necrosis. Kidney Int. 76, 1089-1097 (2009).
14. Ishani, A., Xue, J. L., Himmelfarb, J., Eggers, P. W., Kimmel, P. L., Molitoris, B. A., and Collins, A. J. Acute kidney injury increases risk of ESRD among elderly. J. Am. Soc. Nephrol. 20, 223-228 (2009).
15. Mehta, R. L., Kellum, J. A., Shah, S. V., Molitoris, B. A., Ronco, C., Warnock, D. G., and Levin, A. Acute Kidney Injury Network: report of an initiative to improve outcomes in acute kidney injury. Crit Care 11, R31 (2007).
16. CLSI Protocols for Evaluation of the Linearity of Quantitative Measurement Procedures: A Statistical Approach; Approved Guideline. NCCLS Document EP6-A (ISBN 1-56238-498-8) 2003.
17. CLSI Protocols for Determination of Limits of Detection and Limits of Quantitation; Approved Guideline. CLSI document EP17-A (ISBN 1-56238-551-8), 2004.
18. CLSI Protocols for Evaluation of Precision Performance of Quantitative Measurement Methods; Approved Guideline Second Edition. CLSI Document EP5-A2 (ISBN 1-56238-542-9) 2004.
19. CLSI Protocols for Interference Testing in Clinical Chemistry; Approved Guideline Second Edition. CLSI Document EP7-A2 (ISBN 1-56238-584-4) 2005.
20. ISO 17511:2003. In vitro diagnostic medical devices-Measurement of quantities in biological samples-Metrological traceability of values assigned to calibrator and control materials. ISO, Geneva, Switzerland.
21. CLSI Protocols for Defining, Establishing, and Verifying Reference Intervals in the Clinical Laboratory; Approved Guideline Third Edition. CLSI Document C28-A3 (ISBN 1-56238-682-4) 2008.
22. Guidance for Industry and FDA Staff: Statistical Guidance on Reporting Results from Studies Evaluating Diagnostic Tests (Document Issue Date Mar. 13, 2007). http://www.fda.gov/MedicalDevices/DeviceRegulationandGuidance/GuidanceDocuments/ucm071148.htm

The present invention is further illustrated by the following exemplary embodiments

1. A lateral flow test device for quantitatively detecting multiple analytes in a sample, which device comprises a porous matrix that comprises at least two distinct test locations on said porous matrix, each of said test locations comprising a test reagent that binds to an analyte or another binding reagent that binds to said analyte, or is an analyte or an analyte analog that competes with an analyte in said sample for binding to a binding reagent for said analyte, and said test reagents at said at least two test locations bind to at least two different analytes or different binding reagents that bind to said different analytes, or are different analytes or analyte analogs, wherein a liquid sample flows laterally along said test device and passes said test locations to form a detectable signal to determine amounts of said multiple analytes in said sample.

2. The test device of embodiment 1, wherein the matrix comprises nitrocellulose, glass fiber, polypropylene, polyethylene (preferably of very high molecular weight), polyvinylidene flouride, ethylene vinylacetate, acrylonitrile and/or polytetrafluoro-ethylene.

3. The test device of embodiment 1, wherein the test reagents bind to at least two different analytes.

4. The test device of embodiment 3, wherein the test reagents specifically bind to at least two different analytes.

5. The test device of embodiment 1, wherein the test reagents are different analytes or analyte analogs.

6. The test device of any of the embodiments 1-5, wherein the test reagents are inorganic molecules, organic molecules or a complex thereof.

7. The test device of embodiment 6, wherein the organic molecule is selected from the group consisting of an amino acid, a peptide, a protein, a nucleoside, a nucleotide, an oligonucleotide, a nucleic acid, a vitamin, a monosaccharide, an oligosaccharide, a carbohydrate, a lipid and a complex thereof.

8. The test device of embodiment 7, wherein the protein is an antigen, an antibody or an aptamer.

9. The test device of any of the embodiments 1-8, wherein the matrix is in the form a strip or a circle.

10. The test device of any of the embodiments 1-9, wherein the matrix is a single element or comprises multiple elements.

11. The test device of any of the embodiments 1-10, which further comprises a sample application element upstream from and in fluid communication with the matrix.

12. The test device of any of the embodiments 1-11, which further comprises a liquid absorption element downstream from and in fluid communication with the matrix.

13. The test device of any of the embodiments 1-12, wherein at least a portion of the matrix is supported by a solid backing.

14. The test device of any of the embodiments 1-13, wherein a portion of the matrix, upstream from the test locations, comprises a dried, labeled reagent, the labeled reagent being capable of being moved by a liquid sample and/or a further liquid, e.g., a sample transporting fluid or a washing fluid, to the test locations and/or a positive and/or negative control location to generate a detectable signal.

15. The test device of embodiment 14, which comprises one labeled reagent for one analyte, one labeled reagent for multiple analytes, multiple labeled reagents for one analyte.

16. The test device of embodiment 15, wherein the dried, labeled reagent is located downstream from a sample application place on the test device.

17. The test device of embodiment 15, wherein the dried, labeled reagent is located upstream from a sample application place on the test device.

18. The test device of any of the embodiments 1-17, which further comprises, upstream from the test locations, a conjugate element that comprises a dried, labeled reagent, the labeled reagent being capable of moved by a liquid sample and/or a further liquid to the test locations and/or a positive and/or negative control location to generate a detectable signal.

19. The test device of embodiment 18, wherein the conjugate element is located downstream from a sample application place on the test device.

20. The test device of embodiment 18, wherein the conjugate element is located upstream from a sample application place on the test device.

21. The test device of any of the embodiments 15-20, wherein the labeled reagent binds, and preferably specifically binds, to an analyte in the sample.

22. The test device of any of the embodiments 15-20, which comprises multiple labeled reagents, wherein each of the labeled reagents competes with a different analyte in the sample for binding to a binding reagent for the analyte at a test location.

23. The test device of any of the embodiments 15-22, wherein the label is a soluble label, e.g., a fluorescent label.

24. The test device of any of the embodiments 15-22, wherein the label is a particle label, e.g., a gold or latex particle label.

25. The test device of any of the embodiments 15-24, wherein the labeled reagent is dried in the presence of a material that: a) stabilizes the labeled reagent; b) facilitates solubilization or resuspension of the labeled reagent in a liquid; and/or c) facilitates mobility of the labeled reagent.

26. The test device of embodiment 25, wherein the material is selected from the group consisting of a protein, e.g., a casein or BSA, a peptide, a polysaccharide, a sugar, a polymer, e.g., polyvinylpyrrolidone (PVP-40), a gelatin, a detergent, e.g., Tween-20, and a polyol, e.g., mannitol.

27. The test device of any of the embodiments 1-26, which further comprises a control location comprising means for indicating proper flow of the liquid sample, indicating that the labeled reagent is added to the device, indicating that the labeled reagent is properly solubilized or dispersed, indicating a valid test result, indicating non-specific or unintended specific binding, or indicating heterophilic antibody interference, e.g., human anti-mouse antibody (HAMA) interference, or means for generating a control signal that is compared to signals at the test locations in determining amounts of the multiple analytes.

28. The test device of any of the embodiments 1-27, wherein a sample liquid alone is used to transport the analytes and/or the labeled reagent to the test locations.

29. The test device of any of the embodiments 1-27, wherein a developing liquid is used to transport the analytes and/or the labeled reagent to the test locations.

30. The test device of any of the embodiments 1-29, which further comprises a housing that covers at least a portion of the test device, wherein the housing comprises a sample application port to allow sample application upstream from or to the test locations and an optic opening around the test locations to allow signal detection at the test locations.

31. The test device of embodiment 30, wherein the housing covers the entire test device.

32. The test device of embodiment 30, wherein at least a portion of the sample receiving portion of the matrix or the sample application element is not covered by the housing and a sample or a buffer diluent is applied to the portion of the sample receiving portion of the matrix or the sample application element outside the housing and is then transported to the test locations.

33. The test device of any of the embodiments 30-32, wherein the housing comprises a plastic material.

34. The test device of any of the embodiments 1-33, which are used to for quantitatively detecting 2, 3, 4, 5, 6, 7, 8, 9, 10 or more analytes.

35. The test device of any of the embodiments 1-34, which are used for quantitatively detecting multiple analytes that are diagnostic, prognostic, risk assessment, stratification and/or treatment monitoring markers.

36. The test device of embodiment 35, wherein the analytes are markers for diseases or conditions selected from the group consisting of infectious diseases, parasitic diseases, neoplasms, diseases of the blood and blood-forming organs, disorders involving the immune mechanism, endocrine, nutritional and metabolic diseases, mental and behavioural disorders, diseases of the nervous system, diseases of the eye and adnexam, diseases of the ear and mastoid process, diseases of the circulatory system, diseases of the respiratory system, diseases of the digestive system, diseases of the skin and subcutaneous tissue, diseases of the musculoskeletal system and connective tissue, diseases of the genitourinary system, pregnancy, childbirth and the puerperium, conditions originating in the perinatal period, congenital malformations, deformations, chromosomal abnormalities, injury, poisoning, consequences of external causes, external causes of morbidity and mortality.

37. The test device of embodiment 35, wherein the analytes are markers for acute coronary syndrome (ACS), abdominal pain, cerebrovascular injury, kidney injury, e.g., acute kidney injury or chronic kidney disease, or sepsis.

38. The test device of embodiment 37, wherein the markers for kidney injury are selected from the group consisting of insulin-like growth factor-binding protein 7 (or IGFBP7 or FSTL2 or IBP-7 or IGF-binding protein 7 or IGFBP-7 or IGFBP-7v or IGFBPRP1 or IGFBP-rP1 or MAC25 or MAC-25 or MAC 25 or PGI2-stimulating factor or AGM), Metallopeptidase inhibitor 2 (or CSC-21K or Metalloproteinase inhibitor 2 or TIMP-2 or Tissue inhibitor of metalloproteinases 2 or TIMP2 or TIMP 2), Neutrophil elastase (or Bone marrow serine protease or ELA2 or Elastase-2 or HLE or HNE or Human leukocyte elastase or Medullasin or Neutrophil elastase or PMN-E or PMN elastase or SCN1 or ELANE or elastase neutrophil expressed or elastase 2 or neutrophil-derived elastase or granulocyte-derived elastase or polymorphonuclear elastase or leukocyte elastase), hyaluronic acid (or Hyaluronan or hyaluronate), NGAL, KIM-1, Cystatin C, serum creatinine, L-FABP, IL-18, pi-GST, alph-GST, and Clusterin.

39. The test device of any of the embodiments 1-38, wherein each of the analytes has a concentration ranging from about 1 pg/ml to about 1 μg/ml, e.g., about 1 pg/ml, 10 pg/ml, 100 pg/ml, 1 ng/ml, 2 ng/ml, 3 ng/ml, 3.5 ng/ml, 4 ng/ml, 5 ng/ml, 6 ng/ml, 7 ng/ml, 8 ng/ml, 9 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 950 ng/ml, or higher.

40. The test device of any of the embodiments 1-39, wherein the amount of each of the analytes is determined with a CV ranging from about 0.1% to about 10%.

41. The test device of embodiment 40, wherein each of the analytes has a concentration ranging from about 1 pg/ml to about 1 μg/ml, e.g., about 1 pg/ml, 10 pg/ml, 100 pg/ml, 1 ng/ml, 2 ng/ml, 3 ng/ml, 3.5 ng/ml, 4 ng/ml, 5 ng/ml, 6 ng/ml, 7 ng/ml, 8 ng/ml, 9 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 950 ng/ml, or higher.

42. The test device of any of the embodiments 1-41, which further comprises a liquid container.

43. The test device of any of the embodiments 1-42, which further comprises machine-readable information, e.g., a barcode.

44. The test device of embodiment 43, wherein the barcode comprises lot specific information of the test device, e.g., lot number of the test device.

45. The test device of the embodiment 43, wherein the machine-readable information is comprised in a storage medium, e.g., a RFID device.

46. The test device of embodiment 45, wherein the RFID device comprises lot specific information, information on a liquid control or information to be used for quality control purpose.

47. The test device of any of the embodiments 1-46, wherein a fluorescent conjugate comprising a biological reagent and a fluorescent molecule is used to generate a detectable signal at the test locations, and the fluorescent conjugate and/or the test device further comprises a means for impeding phototoxic degradation of the biological reagent or nonspecific binding of the fluorescent conjugate to the test device or a non-analyte moiety.

48. The test device of the embodiment 43, wherein the means for impeding phototoxic degradation of the biological reagent comprise a cross-linking substance having a long molecular distance, whereby the cross-linking substance links the fluorescent molecule and the biological reagent; a protein; a quencher of singlet oxygen; a quencher of a free radical; a system for depleting oxygen; or a combination thereof.

49. The test device of the embodiment 43, wherein the means for impeding nonspecific binding of the fluorescent conjugate PEG or PEO bound to the fluorescent conjugate.

50. The test device of any of the embodiments 1-48, wherein a liquid has moved laterally along the test device to generate a detectable signal at the test locations.

51. A method for quantitatively detecting multiple analytes in a sample, which method comprises:

a) contacting a liquid sample with the test device of any of the embodiments 1-50, wherein the liquid sample is applied to a site of the test device upstream of the test locations;

b) transporting multiple analytes, if present in the liquid sample, and a labeled reagent to the test locations; and

c) assessing a detectable signal at the test locations to determine the amounts of the multiple analytes in the sample, wherein the amount of each of the analytes is determined.

52. The method of embodiment 51, wherein the amount of each of the analytes is determined with a CV ranging from about 0.1% to about 10%.

53. The method of embodiment 52, wherein each of the analytes has a concentration ranging from about 1 pg/ml to about 1 μg/ml, e.g., 1 pg/ml, 10 pg/ml, 100 pg/ml, about 1 ng/ml, 2 ng/ml, 3 ng/ml, 3.5 ng/ml, 4 ng/ml, 5 ng/ml, 6 ng/ml, 7 ng/ml, 8 ng/ml, 9 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 950 ng/ml, or higher.

54. The method of any of the embodiments 50-53, wherein the liquid sample and the labeled reagent are premixed to form a mixture and the mixture is applied to the test device.

55. The method of embodiment 54, which further comprises a washing step after the mixture is applied to the test device.

56. The method of embodiment 55, wherein the washing step comprises adding a washing liquid after the mixture is applied to the test device.

57. The method of embodiment 45, wherein the test device comprises a liquid container comprising a washing liquid and the washing step comprises releasing the washing liquid from the liquid container.

58. The method of any of the embodiments 50-53, wherein the test device comprises a dried labeled reagent before use and the dried labeled reagent is solubilized or resuspended, and transported to the test locations by the liquid sample.

59. The method of embodiment 58, wherein the dried labeled reagent is located downstream from the sample application site, and the dried labeled reagent is solubilized or resuspended, and transported to the test location by the liquid sample.

60. The method of embodiment 58, wherein the dried labeled reagent is located upstream from the sample application site, and the dried labeled reagent is solubilized or resuspended, and transported to the test location by another liquid.

61. The method of embodiment 58, wherein the labeled reagent is solubilized or resuspended, and transported to the test location by the liquid sample alone.

62. The method of embodiment 58, wherein the multiple analytes and/or labeled reagent are solubilized or resuspended, and transported to the test location by another liquid.

63. The method of any of the embodiments 51-62, wherein the liquid sample is a body fluid sample.

64. The method of embodiment 63, wherein the body fluid sample is selected from the group consisting of a whole blood, a serum, a plasma and a urine sample.

65. The method of any of the embodiments 51-64, wherein the detectable signal is assessed by a reader.

66. The method of embodiment 65, wherein the detectable signal is a fluorescent signal and the fluorescent signal is assessed by a fluorescent reader.

67. The method of embodiment 66, wherein the fluorescent reader is a laser based or a light emitting diode (LED) based fluorescent reader.

68. The method of embodiment 66, wherein the fluorescent reader illuminates at an angle normal to the surface of the test device to excite the fluorescent label at the test locations and detects the fluorescent light at an angle normal to the surface of the test device.

69. The method of embodiment 68, wherein the surface for detection of the fluorescent light in the fluorescent reader is not parallel to the surface of the test device.

70. The method of any of the embodiments 65-69, wherein a light source and a photodetector are positioned at the same side or different sides of the test device.

71. The method of any of the embodiments 65-70, wherein each of the test locations comprises a capture region characterized by a first dimension transverse to the lateral flow direction and a second dimension parallel to the lateral flow direction, and the reader comprises an illumination system operable to focus a beam of light onto an area of the test locations having at least one surface dimension at most equal to smallest of the first and second dimensions of the capture region.

72. The method of any of the embodiments 65-71, wherein the reader comprises a single or multiple photodetectors.

73. The method of any of the embodiments 65-72, wherein the detectable signal is measured at a preset time after the sample is added to the test device.

74. The method of any of the embodiments 65-73, which further comprises comparing the amounts of the multiple analytes to a single threshold or multiple thresholds.

75. The method of embodiment 74, wherein the amount of each of the multiple analytes is compared to a single corresponding threshold or multiple corresponding thresholds.

76. The method of embodiment 74, wherein the amounts of the multiple analytes are used to form a composite amount that is compared to a composite threshold.

77. A system for quantitatively detecting multiple analytes in a sample, which system comprises:

a) a test device of any of the embodiments 1-50; and

b) a reader that comprises a light source and a photodetector to detect a detectable signal.

78. The system of embodiment 71, wherein the reader a fluorescent reader.

79. The system of embodiment 78, wherein the fluorescent reader is a laser based or a light emitting diode (LED) based fluorescent reader.

80. The system of embodiment 79, wherein the fluorescent reader illuminates at an angle normal to the surface of the test device to excite the fluorescent label at the test locations and detects the fluorescent light at an angle normal to the surface of the test device.

81. The system of embodiment 80, wherein the surface for detection of the fluorescent light in the fluorescent reader is not parallel to the surface of the test device.

82. The system of any of the embodiments 77-81, wherein a light source and a photodetector are positioned at the same side or different sides of the test device.

83. The system of any of the embodiments 77-82, wherein each of the test locations comprises a capture region characterized by a first dimension transverse to the lateral flow direction and a second dimension parallel to the lateral flow direction, and the reader comprises an illumination system operable to focus a beam of light onto an area of the test locations having at least one surface dimension at most equal to smallest of the first and second dimensions of the capture region.

84. The system of any of the embodiments 77-83, wherein the reader comprises a single or multiple photodetectors.

85. The system of any of the embodiments 77-84, wherein a detectable signal is measured at a preset time after the sample is added to the test device.

86. The system of any of the embodiments 77-85, wherein the test device further comprises machine-readable information, e.g., a barcode.

87. The system of embodiment 86, wherein the barcode comprises lot specific information of the test device, e.g., lot number of the test device.

88. The system of embodiment 86, wherein the machine-readable information is comprised in a storage medium, e.g., a RFID device.

89. The system of embodiment 88, wherein the RFID device comprises lot specific information, information on a liquid control or information to be used for quality control purpose.

90. A kit for quantitatively detecting multiple analytes in a sample, which kit comprises:

a) a test device of any of the embodiments 1-50; and

b) an instruction for using the test device to quantitatively detect multiple analytes in a sample.

### F. Examples

### Example 1

A fluorescence-based, multiplexed assay system on lateral flow strips is developed. Each test strip in this system includes multiple quantitative assays capable of measuring up to 3 analytes in urine specimens. The test cartridge also includes at least 1 internal positive control. Users read the test cartridge on a fluorescence reader.

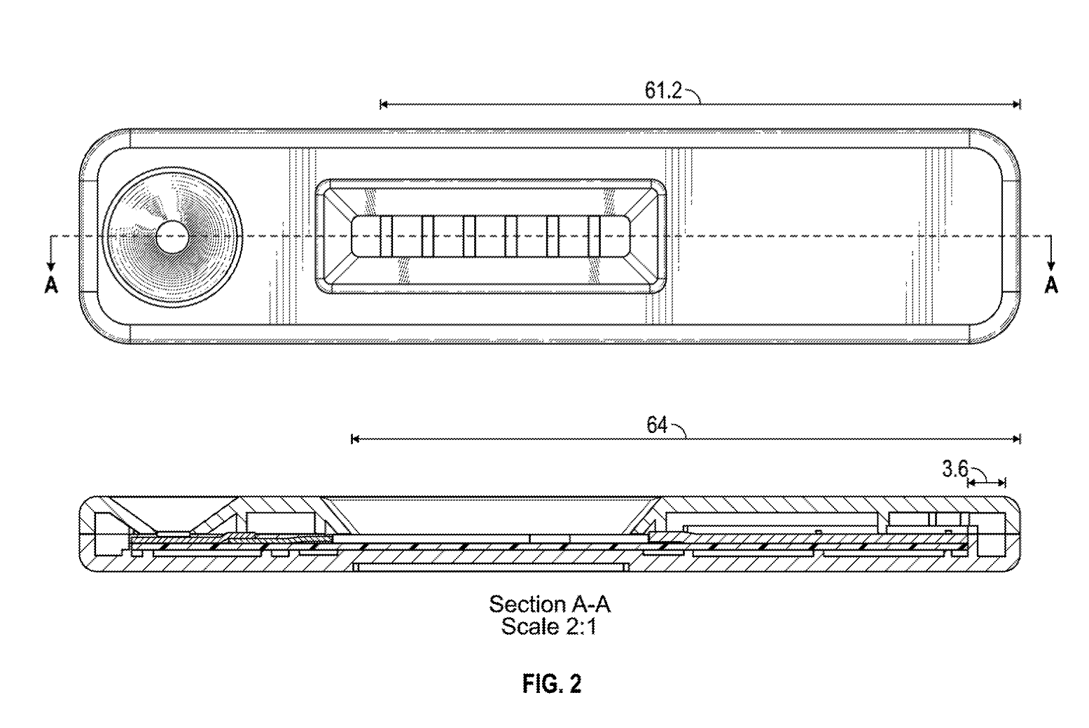

As illustrated in FIGS. 1 and 2, this exemplary lateral flow device contains, from upstream to downstream, a sample receiving pad, a sample treatment pad, a nitrocellulose membrane, and an absorbent pad. The membrane and pads are supported on a plastic backing.

Nitrocellulose Membrane:

The nitrocellulose membrane contains up to five test or control lines (Pos 1 to Pos 5). Each test or control line contains antibodies that have been deposited on to the nitrocellulose membrane. The test and control lines are formatted as show in Table 10.

TABLE 10

| | | | Antibody Striping |
|---|---|---|---|
| Position | Analyte | Antibody | Concentration |
| Pos 1 (6 mm | Empty | | |
| Pos 2 (11 mm) | AN2 (AM-1384) | Ab D | 2 mg/ml |
| | (IGFBP7) | | |
| Pos 3 (16 mm) | AN3 (AM-1051) | Ab 3E10 | 2 mg/ml |
| | (neutrophil | | |
| | elastase) | | |
| Pos 4 (21 mm) | AN1 (AM-1091) | Ab 1 | 2 mg/ml |
| | (TIMP-2) | | |
| Pos 5 (26 mm) | Control | Gt α Ms IgG | 1 mg/ml |

Nitrocellulose Membrane Blocking:

The nitrocellulose membrane containing the test and/or control lines is soaked in a solution containing the following buffering agents, blockers, and preservatives: 10 mM Sodium Phosphate, 0.1% sucrose, 0.1% BSA, 0.2% PVP-40, pH=8. After application of this solution, the membrane is dried at 37° C. for 30 minutes.

Sample Treatment Pad:

The sample treatment pad is a polyester pad that has been soaked in a solution containing the following buffering agents, blockers, and preservatives: 250 mM Tris, 0.25% PVP-40, 0.5% BSA, 0.1% Tween-20, pH=7.19. After application of this solution, the pad is dried at 37° C. for 1 hour.

Sample Receiving Pad:

The sample receiving pad is a cellulose pad that has been soaked in a solution that contains the following buffering and blocking agents: 100 mM Tris, 0.1% Tween-20, 0.25% PVP-40, 0.5% BSA, pH=8.5. After application of this solution, the pad is dried at 37° C. for 1 hour.

The dynamic range and precision of a multiplexed panel of three lateral flow immunoassays were evaluated. The multiplexed panel is composed of on single lateral flow test strip that contains antibodies for the three immunoassays at separate locations within a single nitrocellulose membrane. To evaluate the precision of this multiplexed panel, a series of test samples containing various concentrations of the three immunoassays' target analytes were prepared by spiking purified preparations of the three panel analytes into a running buffer (500 mM Tris, 0.2% 10 G, 0.35% Tween-20, 0.25% PVP-40, pH 8.5). Each test sample was then tested on two strips by placing two test strips into separate polypropylene test tubes, each containing 150 ul of test sample spiked with a mixture of fluorescent antibody conjugates specific to the three immunoassays' target analytes. After allowing the test sample to flow through the strips, the strips were removed from the test tubes and placed in a fluorescent reader (ESE/Qiagen, Germany) where the fluorescent signal for each of the panel assays was measured. These signals were then analyzed to determine the average fluorescent signal as well as coefficient-of-variation (CV) for each test sample and panel analyte.

The test results are shown in the following Tables 11-13.

TABLE 11
Test Results for Analyte 1 (TIMP-2)

| Concentration | Average Test | Standard | |
|---|---|---|---|
| (ng/ml) | Height | Deviation | % CV |
| 56 | 1148.25 | 64.28 | 6% |
| 28 | 698.87 | 2.55 | 0.4% |
| 14 | 398.68 | 9.81 | 2% |
| 7 | 214.26 | 3.86 | 2% |
| 3.5 | 117.90 | 3.01 | 3% |
| 1.75 | 66.63 | 3.89 | 6% |
| 0.875 | 36.13 | 5.68 | 16% |
| 0 | 14.41 | 18.05 | 125% |

TABLE 12
Test Results for Analyte 2 (IGFBP7)

| Concentration | Average Test | Standard | |
|---|---|---|---|
| (ng/ml) | Height | Deviation | % CV |
| 56 | 1380.03 | 22.99 | 2% |
| 28 | 856.18 | 32.04 | 4% |
| 14 | 447.01 | 7.40 | 2% |
| 7 | 231.99 | 21.82 | 9% |
| 3.5 | 119.02 | 10.34 | 9% |
| 1.75 | 71.57 | 11.98 | 17% |
| 0.875 | 27.72 | 8.67 | 31% |
| 0 | 0.00 | 0.00 | #DIV/0! |

TABLE 13
Test Results for Analyte 3 (neutrophil elastase)

| Concentration | Average Test | Standard | |
|---|---|---|---|
| (ng/ml) | Height | Deviation | % CV |
| 56 | 870.94 | 16.16 | 2% |
| 28 | 486.94 | 17.67 | 4% |
| 14 | 263.67 | 1.13 | 0.4% |
| 7 | 117.82 | 7.71 | 7% |
| 3.5 | 69.15 | 2.06 | 3% |
| 1.75 | 40.10 | 9.44 | 24% |
| 0.875 | 32.42 | 8.79 | 27% |
| 0 | 0.00 | 0.00 | #DIV/0! |

### Example 2

The reproducibility of the biomarker assays contained in the NEPHROCHECK™ Test was determined by testing multiple, human urine based control samples (S1, S2, S3) with three different lots of NEPHROCHECK™ Tests (NPK0016, NPK0062, NPK0038). Testing was completed in accordance with the methods described in CLSI guideline EP5-A2¹⁸. Each control sample was evaluated on a total of at least 240 tests from three different lots of test kits (80 tests per lot). These data were collected over 40 separate runs that were conducted twice a day over at least 20 total days of testing. Study results were analyzed as described in CLSI guideline EP5-A2 to determine within-run, run-to-run, and total assay CV's. The raw data and CV's from these studies are provided in Tables 14 and 15 below.

TABLE 14
The tabulated results for each biomarker and levels
tested (S1, S2, and S3). The table lists results
(ng/ml) from each replicate, run and day

| NPK0038 | AM-1091 (S1) | |
|---|---|---|
| Day | Run 1 | Run 2 |

| | | | | |
|---|---|---|---|---|
| 1 | 2.5 | 3.2 | 2.5 | 2.4 |
| 2 | 2.5 | 2.4 | 2.6 | 2.9 |
| 3 | 2.8 | 2.6 | 2.8 | 2.6 |
| 4 | 2.5 | 2.7 | 2.4 | 3.2 |
| 5 | 2.9 | 2.2 | 2.4 | 3.1 |
| 6 | 2.9 | 2.6 | 3.0 | 2.6 |
| 7 | 2.7 | 2.8 | 2.4 | 2.6 |
| 8 | 3.0 | 2.8 | 3.1 | 2.9 |
| 9 | 2.5 | 3.1 | 2.7 | 3.0 |
| 10 | 2.3 | 2.9 | 2.4 | 2.5 |
| 11 | 3.1 | 2.5 | 2.6 | 2.7 |
| 12 | 2.9 | 3.1 | 2.6 | 3.0 |
| 13 | 2.5 | 3.1 | 2.5 | Excluded |
| 14 | 2.4 | 3.1 | 2.4 | 2.9 |
| 15 | 3.1 | 3.3 | 2.7 | 3.2 |
| 16 | 2.8 | 2.9 | 2.9 | 2.7 |
| 17 | 2.3 | 2.7 | 3.0 | 2.4 |
| 18 | 2.9 | 2.9 | 3.0 | 2.4 |
| 19 | 3.0 | 2.4 | 2.6 | 2.5 |
| 20 | 2.9 | 2.4 | 2.7 | 2.5 |
| 21 | 2.4 | 2.6 | 2.8 | 2.5 |

| NPK0038 | AM-1091 (S2) | |
|---|---|---|
| Day | Run 1 | Run 2 |

| | | | | |
|---|---|---|---|---|
| 1 | 148.5 | 123.2 | 134.8 | 140.9 |
| 2 | 126.8 | 135.5 | 137.9 | 130.6 |
| 3 | 143.4 | 135.8 | 131.5 | 144.4 |
| 4 | 126.1 | 144.2 | 138.5 | 152.9 |
| 5 | 130.1 | 145.1 | 141.6 | 136.5 |
| 6 | 132.3 | 155.7 | 143.1 | 155.8 |
| 7 | Excluded | 132.4 | 143.5 | 124.4 |
| 8 | 133.0 | 132.1 | 146.8 | 132.9 |
| 9 | 139.9 | 140.7 | 141.9 | 139.0 |
| 10 | 146.6 | 132.1 | 165.4 | 133.1 |
| 11 | 136.5 | 139.1 | 151.2 | 128.7 |
| 12 | 142.1 | 133.4 | 151.6 | 128.9 |
| 13 | 151.0 | 130.0 | 133.2 | 146.2 |
| 14 | 133.9 | 152.3 | 136.7 | 147.0 |
| 15 | 146.3 | 136.1 | 128.9 | 166.1 |
| 16 | 142.1 | 137.2 | 143.1 | 142.0 |
| 17 | 141.4 | 130.7 | 159.3 | 129.8 |
| 18 | 133.9 | 140.5 | 133.8 | 147.1 |
| 19 | 131.1 | 139.9 | 126.3 | 145.6 |
| 20 | 127.7 | 138.3 | 150.5 | 138.9 |
| 21 | 131.6 | 143.8 | 146.8 | 131.4 |

| NPK0038 | AM-1091 (S3) | |
|---|---|---|
| Day | Run 1 | Run 2 |

| | | | | |
|---|---|---|---|---|
| 1 | 298.2 | 238.6 | 273.3 | 273.4 |
| 2 | 284.7 | 260.7 | 257.9 | 298.5 |
| 3 | 271.5 | 291.0 | 289.4 | 271.1 |
| 4 | 274.9 | 274.6 | 310.1 | 249.1 |
| 5 | 293.0 | 253.3 | 269.0 | 299.0 |
| 6 | 321.7 | 278.3 | 257.3 | 322.5 |
| 7 | 254.5 | 288.6 | 271.4 | 264.5 |
| 8 | 270.1 | 257.0 | 276.8 | 270.8 |
| 9 | 256.1 | 256.1 | 274.4 | 278.6 |
| 10 | 281.0 | 252.2 | 261.9 | 303.5 |
| 11 | 307.2 | 267.7 | 303.1 | 264.3 |
| 12 | 291.5 | 263.5 | 264.1 | 276.7 |
| 13 | 301.6 | 255.7 | 254.6 | 295.4 |
| 14 | 259.8 | 310.0 | 267.8 | 292.3 |
| 15 | 285.1 | 273.2 | 254.8 | 313.6 |
| 16 | 287.0 | 274.9 | 276.7 | 271.5 |
| 17 | 309.8 | 264.8 | 312.2 | 257.6 |
| 18 | 268.1 | 291.3 | 259.4 | 308.6 |
| 19 | 269.8 | 280.9 | 312.4 | 249.8 |
| 20 | 271.4 | 272.8 | 307.4 | 273.3 |
| 21 | 269.8 | 244.8 | 247.2 | 279.0 |

| NPK0062 | AM-1091 (S1) | |
|---|---|---|
| Day | Run 1 | Run 2 |

| | | | | |
|---|---|---|---|---|
| 1 | 3.1 | 2.5 | 2.7 | 2.8 |
| 2 | 2.7 | 2.5 | 2.8 | 2.4 |
| 3 | 3.2 | 2.9 | 2.7 | 3.3 |
| 4 | 2.6 | 3.4 | 2.4 | 2.7 |
| 5 | 2.8 | 3.2 | 2.8 | 3.0 |
| 6 | 2.8 | 3.0 | 2.9 | 2.6 |
| 7 | 2.6 | 2.6 | 2.7 | 2.7 |
| 8 | 3.2 | 2.7 | 2.4 | 2.9 |
| 9 | 3.0 | 3.1 | 2.7 | 3.4 |
| 10 | 2.9 | 2.8 | 2.6 | 2.9 |
| 11 | 2.4 | 3.2 | 2.7 | 2.7 |
| 12 | 2.6 | 3.0 | 3.3 | 2.5 |
| 13 | 2.0 | 2.9 | 2.7 | 2.9 |
| 14 | 2.8 | 2.9 | 2.9 | 2.6 |
| 15 | 2.9 | 3.3 | 2.9 | 3.1 |
| 16 | 2.9 | 3.1 | 2.9 | 2.9 |
| 17 | 2.9 | 2.2 | 3.2 | 2.4 |
| 18 | 2.8 | 2.9 | 2.7 | 2.6 |
| 19 | 2.7 | 3.0 | 2.6 | 3.3 |
| 20 | 3.0 | 3.2 | 2.7 | 3.0 |
| 21 | 3.0 | 3.3 | 3.0 | 2.8 |

| NPK0062 | AM-1091 (S2) | |
|---|---|---|
| Day | Run 1 | Run 2 |

| | | | | |
|---|---|---|---|---|
| 1 | 150.6 | 131.9 | 147.1 | 128.9 |
| 2 | 156.9 | 131.0 | 147.7 | 126.9 |
| 3 | 140.2 | 149.9 | 130.2 | 145.5 |
| 4 | 139.9 | 142.4 | 127.3 | 147.5 |
| 5 | 136.8 | 162.2 | 142.8 | 154.7 |
| 6 | 150.8 | 128.8 | 149.3 | 128.7 |
| 7 | 153.3 | 135.8 | 142.6 | 147.8 |
| 8 | 139.7 | 146.7 | 146.5 | 139.2 |
| 9 | 140.9 | 145.6 | 131.0 | 141.3 |
| 10 | 150.5 | 130.5 | 144.7 | 145.0 |
| 11 | 132.7 | 133.8 | 143.1 | 155.3 |
| 12 | 149.6 | 136.9 | 133.8 | 139.1 |
| 13 | 125.6 | 140.1 | 145.3 | 132.8 |
| 14 | 145.7 | 142.3 | 135.8 | 160.3 |
| 15 | 135.2 | 150.3 | 130.9 | 152.7 |
| 16 | 137.8 | 153.7 | 142.6 | 143.2 |
| 17 | 131.1 | 150.3 | 138.5 | 165.4 |
| 18 | 136.4 | 135.2 | 129.2 | 148.3 |
| 19 | 136.7 | 146.2 | 145.0 | 165.9 |
| 20 | 135.1 | 140.3 | 124.8 | 158.1 |
| 21 | 131.4 | 132.3 | 143.1 | 131.4 |

| NPK0062 | AM-1091 (S3) | |
|---|---|---|
| Day | Run 1 | Run 2 |

| | | | | |
|---|---|---|---|---|
| 1 | 320.0 | 263.3 | 277.6 | 292.1 |
| 2 | 262.5 | 309.7 | 258.4 | 291.2 |
| 3 | 275.6 | 315.9 | 277.1 | 281.9 |
| 4 | 260.8 | 312.6 | 262.3 | 298.0 |
| 5 | 283.1 | 306.5 | 304.4 | 249.5 |
| 6 | 312.9 | 277.4 | 252.7 | 319.8 |
| 7 | 264.9 | 291.9 | 298.0 | 251.3 |
| 8 | 257.0 | 301.1 | 305.4 | 259.9 |
| 9 | 270.8 | 301.5 | 279.2 | 286.5 |
| 10 | 273.3 | 334.6 | 311.5 | 286.6 |
| 11 | 239.7 | 266.3 | 326.0 | 272.8 |
| 12 | 283.0 | 272.2 | 288.8 | 250.6 |
| 13 | 250.6 | 278.2 | 274.4 | 278.3 |
| 14 | 291.8 | 315.1 | 258.1 | 309.8 |
| 15 | 313.2 | 274.0 | 257.6 | 285.5 |
| 16 | 284.8 | 309.5 | 275.4 | 288.6 |
| 17 | 261.7 | 281.7 | 292.1 | 288.9 |
| 18 | 266.8 | 269.8 | 274.1 | 310.0 |
| 19 | 265.4 | 292.5 | 301.1 | 301.9 |
| 20 | 269.3 | 280.3 | 299.5 | 278.9 |
| 21 | 262.6 | 267.5 | 277.0 | 266.5 |

| NPK0016 | AM-1091 (S1) | |
|---|---|---|
| Day | Run 1 | Run 2 |

| | | | | |
|---|---|---|---|---|
| 1 | 2.2 | 2.4 | 2.7 | 2.3 |
| 2 | 2.4 | 2.1 | 2.4 | 2.2 |
| 3 | 2.6 | 2.4 | 2.8 | 2.4 |
| 4 | 2.2 | 2.6 | 2.1 | 2.9 |
| 5 | 2.6 | 2.3 | 2.2 | 2.3 |
| 6 | 2.5 | 2.4 | 2.1 | 2.4 |
| 7 | 2.0 | 2.6 | 2.6 | 2.2 |
| 8 | 2.4 | 2.8 | 2.5 | 2.6 |
| 9 | 2.1 | 2.3 | 2.2 | 2.5 |
| 10 | 2.6 | 2.3 | 2.1 | 2.6 |
| 11 | 2.2 | 2.3 | 2.5 | 2.8 |
| 12 | 2.4 | 2.2 | 2.3 | 2.3 |
| 13 | 2.5 | 2.7 | 2.5 | 2.9 |
| 14 | 2.5 | 2.5 | 2.5 | 2.3 |
| 15 | 2.2 | 2.6 | 2.4 | 2.2 |
| 16 | 2.4 | 2.5 | 2.5 | 2.5 |
| 17 | 2.2 | 2.4 | 2.6 | 2.2 |
| 18 | 2.5 | 2.6 | 2.3 | 2.6 |
| 19 | 2.2 | 2.7 | 2.3 | 2.4 |
| 20 | 2.6 | 2.6 | 2.5 | 2.9 |
| 21 | 2.3 | 2.4 | 2.2 | 2.5 |

| NPK0016 | AM-1091 (S2) | |
|---|---|---|
| Day | Run 1 | Run 2 |

| | | | | |
|---|---|---|---|---|
| 1 | 115.2 | 138.4 | 125.2 | 123.1 |
| 2 | 114.2 | 143.0 | 114.1 | 145.4 |
| 3 | 131.6 | 120.8 | 119.4 | 137.2 |
| 4 | 122.9 | 134.6 | 139.9 | 123.7 |
| 5 | 137.0 | 123.8 | 127.1 | 131.2 |
| 6 | 142.3 | 135.4 | 123.5 | 123.3 |
| 7 | 148.1 | 128.5 | 132.1 | 131.2 |
| 8 | 130.0 | 130.6 | 136.5 | 121.6 |
| 9 | 126.8 | 117.6 | 131.4 | 118.6 |
| 10 | 134.8 | 121.2 | 124.6 | 139.3 |
| 11 | 134.6 | 122.6 | 119.6 | 139.6 |
| 12 | 131.3 | 128.4 | 136.1 | 121.9 |
| 13 | 114.7 | 145.1 | 119.7 | 147.1 |
| 14 | 139.8 | 124.6 | 117.9 | 150.3 |
| 15 | 125.8 | 121.5 | 124.3 | 142.4 |
| 16 | 128.7 | 129.1 | 124.5 | 128.2 |
| 17 | 144.4 | 119.7 | 122.4 | 137.0 |
| 18 | 125.7 | 135.2 | 136.0 | 127.1 |
| 19 | 133.2 | 130.3 | 137.9 | 134.0 |
| 20 | 125.7 | 130.5 | 133.0 | 132.0 |
| 21 | 127.8 | 125.1 | 130.5 | 124.7 |

| NPK0016 | AM-1091 (S3) | |
|---|---|---|
| Day | Run 1 | Run 2 |

| | | | | |
|---|---|---|---|---|
| 1 | 237.9 | 264.3 | 244.2 | 271.1 |
| 2 | 270.7 | 234.2 | 223.7 | 252.1 |
| 3 | 242.7 | 259.0 | 252.9 | 254.9 |
| 4 | 250.7 | 271.3 | 265.1 | 303.0 |
| 5 | 283.7 | 235.2 | 243.8 | 271.4 |
| 6 | 270.2 | 249.8 | 273.3 | 250.5 |
| 7 | 290.4 | 241.6 | 279.2 | 246.9 |
| 8 | 255.9 | 263.2 | 240.2 | 248.8 |
| 9 | 240.6 | 249.6 | 245.6 | 262.7 |
| 10 | 234.4 | 270.6 | 230.7 | 265.2 |
| 11 | 253.5 | 246.0 | 298.6 | 244.0 |
| 12 | 257.3 | 263.5 | 284.6 | 249.1 |
| 13 | 270.4 | 281.9 | 268.2 | 261.2 |
| 14 | 271.4 | 245.4 | 255.8 | 241.5 |
| 15 | 256.6 | 264.1 | 251.1 | 273.0 |
| 16 | 248.1 | 248.0 | 247.2 | 266.8 |
| 17 | 282.5 | 253.6 | 251.1 | 283.2 |
| 18 | 262.6 | 264.5 | 292.3 | 245.2 |
| 19 | 284.1 | 261.3 | 268.0 | 269.7 |
| 20 | 257.4 | 276.3 | 264.9 | 282.4 |
| 21 | 258.8 | 257.7 | 239.1 | 242.9 |

| NPK0038 | AM-1384 (S1) | |
|---|---|---|
| Day | Run 1 | Run 2 |

| | | | | |
|---|---|---|---|---|
| 1 | 34.2 | 40.3 | 37.5 | 33.1 |
| 2 | 34.6 | 37.1 | 33.8 | 36.2 |
| 3 | 39.1 | 33.9 | 37.8 | 35.6 |
| 4 | 36.5 | 34.7 | 35.2 | 40.0 |
| 5 | 40.3 | 31.2 | 35.2 | 39.5 |
| 6 | 40.1 | 32.8 | 39.2 | 35.0 |
| 7 | 38.0 | 36.0 | 34.3 | 34.9 |
| 8 | 38.0 | 37.2 | 38.5 | 37.6 |
| 9 | 34.5 | 38.5 | 37.4 | 39.9 |
| 10 | 34.6 | 40.4 | 36.3 | 36.4 |
| 11 | 42.3 | 32.6 | 37.0 | 38.0 |
| 12 | 38.6 | 39.6 | 35.9 | 41.5 |
| 13 | 35.9 | 39.0 | 35.9 | Excluded |
| 14 | 34.6 | 40.7 | 35.5 | 38.4 |
| 15 | 37.2 | 40.9 | 37.2 | 41.1 |
| 16 | 37.0 | 38.0 | 36.8 | 38.4 |
| 17 | 33.3 | 39.4 | 40.0 | 36.2 |
| 18 | 37.7 | 37.2 | 40.5 | 35.9 |
| 19 | 38.4 | 35.3 | 35.4 | 37.6 |
| 20 | 36.2 | 35.3 | 37.6 | 38.5 |
| 21 | 37.4 | 37.6 | 37.7 | 37.3 |

| NPK0038 | AM-1384 (S2) | |
|---|---|---|
| Day | Run 1 | Run 2 |

| | | | | |
|---|---|---|---|---|
| 1 | 220.8 | 185.7 | 196.4 | 209.8 |
| 2 | 189.7 | 204.6 | 206.5 | 191.7 |
| 3 | 215.7 | 206.4 | 196.4 | 216.0 |
| 4 | 194.5 | 217.4 | 213.9 | 233.1 |
| 5 | 190.2 | 209.6 | 207.7 | 204.3 |
| 6 | 201.7 | 228.4 | 212.4 | 228.3 |
| 7 | Excluded | 202.1 | 207.4 | 191.6 |
| 8 | 215.5 | 210.7 | 215.8 | 204.1 |
| 9 | 216.8 | 214.4 | 213.9 | 216.0 |
| 10 | 222.2 | 209.5 | 239.0 | 201.3 |
| 11 | 210.5 | 210.6 | 228.1 | 196.2 |
| 12 | 211.5 | 205.6 | 229.0 | 202.2 |
| 13 | 224.2 | 197.7 | 204.6 | 214.7 |
| 14 | 210.5 | 229.7 | 205.8 | 218.3 |
| 15 | 221.7 | 208.5 | 198.7 | 237.4 |
| 16 | 211.2 | 211.7 | 220.1 | 212.9 |
| 17 | 217.8 | 202.2 | 232.5 | 200.5 |
| 18 | 198.7 | 207.5 | 202.2 | 214.8 |
| 19 | 198.4 | 215.1 | 194.0 | 214.6 |
| 20 | 202.5 | 209.2 | 222.2 | 211.3 |
| 21 | 204.9 | 222.0 | 220.6 | 206.0 |

| NPK0038 | AM-1384 (S3) | |
|---|---|---|
| Day | Run 1 | Run 2 |

| | | | | |
|---|---|---|---|---|
| 1 | 473.2 | 428.8 | 465.5 | 468.3 |
| 2 | 487.8 | 457.1 | 459.1 | 513.4 |
| 3 | 463.1 | 489.1 | 475.5 | 464.3 |
| 4 | 483.6 | 486.0 | 535.1 | 445.0 |
| 5 | 506.8 | 452.5 | 467.4 | 489.9 |
| 6 | 520.0 | 473.1 | 458.4 | 531.5 |
| 7 | 437.6 | 481.4 | 469.5 | 453.5 |
| 8 | 459.6 | 468.2 | 466.0 | 475.9 |
| 9 | 456.2 | 432.4 | 475.7 | 478.8 |
| 10 | 497.2 | 461.7 | 448.9 | 502.0 |
| 11 | 497.4 | 461.3 | 516.8 | 472.9 |
| 12 | 501.4 | 472.1 | 482.2 | 503.7 |
| 13 | 510.3 | 466.7 | 450.5 | 489.4 |
| 14 | 460.0 | 528.8 | 472.5 | 506.6 |
| 15 | 487.9 | 481.7 | 456.8 | 528.9 |
| 16 | 493.4 | 479.1 | 477.6 | 477.4 |
| 17 | 524.5 | 463.0 | 522.6 | 466.7 |
| 18 | 475.6 | 484.0 | 456.0 | 512.9 |
| 19 | 469.5 | 488.8 | 513.2 | 447.7 |
| 20 | 479.9 | 469.0 | 529.9 | 497.4 |
| 21 | 474.5 | 448.7 | 438.3 | 470.1 |

| NPK0062 | AM-1384 (S1) | |
|---|---|---|
| Day | Run 1 | Run 2 |

| | | | | |
|---|---|---|---|---|
| 1 | 41.0 | 37.5 | 40.6 | 37.6 |
| 2 | 39.3 | 35.6 | 38.1 | 37.0 |
| 3 | 39.5 | 38.4 | 35.7 | 41.1 |
| 4 | 37.9 | 42.9 | 36.4 | 37.9 |
| 5 | 43.3 | 43.6 | 37.9 | 39.7 |
| 6 | 37.0 | 40.1 | 36.8 | 35.9 |
| 7 | 37.9 | 33.6 | 39.5 | 38.0 |
| 8 | 38.9 | 38.2 | 36.0 | 37.2 |
| 9 | 40.2 | 40.2 | 37.2 | 42.2 |
| 10 | 38.2 | 41.3 | 37.5 | 39.9 |
| 11 | 31.9 | 41.6 | 39.7 | 36.6 |
| 12 | 37.0 | 39.8 | 42.3 | 31.4 |
| 13 | 32.6 | 39.8 | 35.1 | 38.7 |
| 14 | 40.1 | 40.0 | 39.8 | 44.3 |
| 15 | 37.2 | 45.4 | 37.7 | 40.8 |
| 16 | 41.7 | 37.2 | 38.9 | 37.7 |
| 17 | 37.0 | 33.5 | 41.4 | 35.5 |
| 18 | 37.8 | 39.7 | 39.3 | 36.1 |
| 19 | 36.8 | 41.2 | 36.2 | 41.7 |
| 20 | 40.7 | 38.7 | 37.4 | 39.7 |
| 21 | 39.0 | 39.1 | 39.7 | 38.5 |

| NPK0062 | AM-1384 (S2) | |
|---|---|---|
| Day | Run 1 | Run 2 |

| | | | | |
|---|---|---|---|---|
| 1 | 219.5 | 189.2 | 214.3 | 195.9 |
| 2 | 227.8 | 194.7 | 215.8 | 185.6 |
| 3 | 208.2 | 221.8 | 192.8 | 214.5 |
| 4 | 210.7 | 209.6 | 198.3 | 231.0 |
| 5 | 200.0 | 243.9 | 214.4 | 223.3 |
| 6 | 221.2 | 193.2 | 218.6 | 182.1 |
| 7 | 221.6 | 200.8 | 209.8 | 220.8 |
| 8 | 201.6 | 219.1 | 207.6 | 206.4 |
| 9 | 210.4 | 208.4 | 197.2 | 212.9 |
| 10 | 227.6 | 205.5 | 194.3 | 206.0 |
| 11 | 198.3 | 194.3 | 217.6 | 222.2 |
| 12 | 215.2 | 202.8 | 198.6 | 201.8 |
| 13 | 192.5 | 215.0 | 221.4 | 202.6 |
| 14 | 211.1 | 215.1 | 206.3 | 236.5 |
| 15 | 197.1 | 216.8 | 199.4 | 222.0 |
| 16 | 200.7 | 219.2 | 207.0 | 206.9 |
| 17 | 192.7 | 218.9 | 207.5 | 236.6 |
| 18 | 203.9 | 198.8 | 193.9 | 209.8 |
| 19 | 200.8 | 212.3 | 219.2 | 239.6 |
| 20 | 203.7 | 213.6 | 194.1 | 233.2 |
| 21 | 195.7 | 190.0 | 215.5 | 206.6 |

| NPK0062 | AM-1384 (S3) | |
|---|---|---|
| Day | Run 1 | Run 2 |

| | | | | |
|---|---|---|---|---|
| 1 | 504.4 | 463.9 | 456.0 | 492.0 |
| 2 | 431.6 | 502.4 | 444.2 | 497.0 |
| 3 | 441.0 | 500.9 | 472.3 | 448.5 |
| 4 | 442.1 | 537.3 | 463.2 | 527.9 |
| 5 | 476.9 | 514.3 | 510.0 | 423.6 |
| 6 | 504.3 | 427.9 | 417.6 | 510.5 |
| 7 | 443.7 | 498.2 | 497.7 | 429.2 |
| 8 | 437.7 | 522.9 | 515.6 | 431.8 |
| 9 | 447.8 | 506.0 | 473.9 | 464.4 |
| 10 | 485.3 | 569.9 | 495.7 | 448.1 |
| 11 | 429.4 | 460.7 | 535.3 | 476.4 |
| 12 | 461.4 | 447.0 | 501.4 | 432.5 |
| 13 | 439.1 | 477.2 | 477.0 | 463.2 |
| 14 | 485.8 | 501.2 | 452.6 | 525.4 |
| 15 | 509.7 | 463.7 | 436.4 | 482.7 |
| 16 | 469.9 | 496.9 | 468.6 | 490.8 |
| 17 | 443.6 | 457.4 | 481.1 | 482.8 |
| 18 | 451.9 | 448.5 | 473.4 | 526.7 |
| 19 | 449.0 | 493.0 | 475.4 | 472.3 |
| 20 | 468.9 | 480.1 | 512.9 | 463.7 |
| 21 | 455.8 | 475.7 | 479.8 | 458.4 |

| NPK0016 | AM-1384 (S1) | |
|---|---|---|
| Day | Run 1 | Run 2 |

| | | | | |
|---|---|---|---|---|
| 1 | 32.2 | 37.2 | 36.3 | 34.1 |
| 2 | 36.5 | 32.3 | 37.3 | 33.4 |
| 3 | 36.1 | 35.3 | 38.2 | 34.7 |
| 4 | 33.4 | 38.8 | 36.0 | 41.1 |
| 5 | 35.1 | 37.4 | 35.7 | 36.5 |
| 6 | 36.0 | 35.9 | 36.8 | 35.9 |
| 7 | 32.7 | 38.8 | 36.1 | 34.5 |
| 8 | 35.6 | 37.1 | 36.8 | 35.6 |
| 9 | 34.0 | 36.8 | 34.7 | 38.1 |
| 10 | 39.3 | 36.2 | 32.1 | 36.8 |
| 11 | 34.6 | 37.6 | 37.5 | 42.1 |
| 12 | 38.0 | 36.6 | 37.5 | 33.9 |
| 13 | 37.1 | 40.9 | 35.1 | 43.5 |
| 14 | 38.5 | 37.4 | 40.4 | 33.4 |
| 15 | 36.0 | 37.0 | 38.1 | 34.3 |
| 16 | 37.2 | 37.5 | 36.5 | 37.1 |
| 17 | 37.0 | 38.5 | 40.2 | 36.0 |
| 18 | 36.4 | 36.6 | 36.5 | 39.8 |
| 19 | 35.1 | 40.1 | 37.0 | 37.4 |
| 20 | 37.4 | 37.7 | 38.5 | 42.0 |
| 21 | 36.5 | 37.5 | 35.0 | 36.3 |

| NPK0016 | AM-1384 (S2) | |
|---|---|---|
| Day | Run 1 | Run 2 |

| | | | | |
|---|---|---|---|---|
| 1 | 178.5 | 206.2 | 192.9 | 185.5 |
| 2 | 175.6 | 212.2 | 176.0 | 213.9 |
| 3 | 195.7 | 181.0 | 180.5 | 201.4 |
| 4 | 185.0 | 198.9 | 215.3 | 190.3 |
| 5 | 200.9 | 194.5 | 188.7 | 192.8 |
| 6 | 213.1 | 202.4 | 183.1 | 186.2 |
| 7 | 219.5 | 195.0 | 201.7 | 199.7 |
| 8 | 193.6 | 195.3 | 201.2 | 188.9 |
| 9 | 194.0 | 182.2 | 198.2 | 185.7 |
| 10 | 208.2 | 184.6 | 179.3 | 202.4 |
| 11 | 206.6 | 187.9 | 184.2 | 216.2 |
| 12 | 201.9 | 192.2 | 210.8 | 188.6 |
| 13 | 183.8 | 217.6 | 186.4 | 225.2 |
| 14 | 216.7 | 196.8 | 182.5 | 226.2 |
| 15 | 190.1 | 190.0 | 186.8 | 213.2 |
| 16 | 200.9 | 198.6 | 187.2 | 195.2 |
| 17 | 212.7 | 185.7 | 185.8 | 212.4 |
| 18 | 190.6 | 199.4 | 211.5 | 195.5 |
| 19 | 204.0 | 196.3 | 209.3 | 204.4 |
| 20 | 196.6 | 197.9 | 208.4 | 207.4 |
| 21 | 193.2 | 189.0 | 202.4 | 185.3 |

| NPK0016 | AM-1384 (S3) | |
|---|---|---|
| Day | Run 1 | Run 2 |

| | | | | |
|---|---|---|---|---|
| 1 | 407.0 | 464.0 | 420.5 | 449.6 |
| 2 | 447.1 | 387.0 | 381.5 | 420.9 |
| 3 | 406.3 | 426.0 | 432.5 | 434.1 |
| 4 | 419.0 | 442.6 | 446.9 | 520.9 |
| 5 | 472.7 | 422.4 | 417.7 | 469.5 |
| 6 | 461.6 | 419.5 | 470.9 | 428.6 |
| 7 | 494.1 | 417.6 | 471.3 | 413.7 |
| 8 | 427.8 | 442.0 | 406.8 | 419.7 |
| 9 | 400.1 | 426.2 | 417.1 | 439.5 |
| 10 | 400.6 | 464.2 | 381.7 | 428.3 |
| 11 | 451.6 | 438.0 | 543.0 | 441.4 |
| 12 | 442.1 | 453.1 | 493.6 | 437.1 |
| 13 | 479.2 | 493.8 | 490.2 | 469.6 |
| 14 | 477.0 | 424.8 | 455.3 | 439.4 |
| 15 | 439.6 | 440.7 | 424.8 | 451.3 |
| 16 | 425.7 | 420.2 | 433.4 | 429.5 |
| 17 | 493.0 | 428.0 | 441.2 | 479.7 |
| 18 | 432.4 | 451.3 | 499.6 | 440.2 |
| 19 | 477.4 | 455.3 | 463.3 | 456.4 |
| 20 | 458.8 | 461.5 | 442.8 | 488.1 |
| 21 | 442.3 | 443.1 | 402.0 | 410.7 |

TABLE 15
The calculated within-run, run to run, and Total
CV for the each biomarker, sample level, and lot.

| | | | | |
|---|---|---|---|---|
| | | | | Avg. Conc. |
| Variation | Level | Lot | CV | (ng/mL) |

AM-1091

| | | | | |
|---|---|---|---|---|
| Within-Run | S1 | NPK0016 | 9.3% | 2.4 |
| Within-Run | S1 | NPK0062 | 10.8% | 2.8 |
| Within-Run | S1 | NPK0038 | 10.7% | 2.7 |
| Within-Run | S2 | NPK0016 | 8.3% | 129.7 |
| Within-Run | S2 | NPK0062 | 8.0% | 141.7 |
| Within-Run | S2 | NPK0038 | 8.0% | 139.5 |
| Within-Run | S3 | NPK0016 | 7.0% | 259.4 |
| Within-Run | S3 | NPK0062 | 8.6% | 283.2 |
| Within-Run | S3 | NPK0038 | 9.0% | 277.3 |
| Run to Run | S1 | NPK0016 | 0.0% | 2.4 |
| Run to Run | S1 | NPK0062 | 0.0% | 2.8 |
| Run to Run | S1 | NPK0038 | 0.0% | 2.7 |
| Run to Run | S2 | NPK0016 | 0.0% | 129.7 |
| Run to Run | S2 | NPK0062 | 0.0% | 141.7 |
| Run to Run | S2 | NPK0038 | 0.0% | 139.5 |
| Run to Run | S3 | NPK0016 | 0.0% | 259.4 |
| Run to Run | S3 | NPK0062 | 0.0% | 283.2 |
| Run to Run | S3 | NPK0038 | 0.0% | 277.3 |
| Day to Day | S1 | NPK0016 | 3.0% | 2.4 |
| Day to Day | S1 | NPK0062 | 3.4% | 2.8 |
| Day to Day | S1 | NPK0038 | 4.0% | 2.7 |
| Day to Day | S2 | NPK0016 | 0.8% | 129.7 |
| Day to Day | S2 | NPK0062 | 1.3% | 141.7 |
| Day to Day | S2 | NPK0038 | 1.2% | 139.5 |
| Day to Day | S3 | NPK0016 | 2.5% | 259.4 |
| Day to Day | S3 | NPK0062 | 0.0% | 283.2 |
| Day to Day | S3 | NPK0038 | 2.2% | 277.3 |
| Total Precision | S1 | NPK0016 | 9.7% | 2.4 |
| Total Precision | S1 | NPK0062 | 11.3% | 2.8 |
| Total Precision | S1 | NPK0038 | 11.4% | 2.7 |
| Total Precision | S2 | NPK0016 | 8.4% | 129.7 |
| Total Precision | S2 | NPK0062 | 8.1% | 141.7 |
| Total Precision | S2 | NPK0038 | 8.1% | 139.5 |
| Total Precision | S3 | NPK0016 | 7.4% | 259.4 |
| Total Precision | S3 | NPK0062 | 8.6% | 283.2 |
| Total Precision | S3 | NPK0038 | 9.3% | 277.3 |

AM-1384

| | | | | |
|---|---|---|---|---|
| Within-Run | S1 | NPK0016 | 6.6% | 36.8 |
| Within-Run | S1 | NPK0062 | 7.5% | 38.6 |
| Within-Run | S1 | NPK0038 | 7.7% | 37.1 |
| Within-Run | S2 | NPK0016 | 7.3% | 197.1 |
| Within-Run | S2 | NPK0062 | 7.0% | 209.3 |
| Within-Run | S2 | NPK0038 | 6.3% | 210.7 |
| Within-Run | S3 | NPK0016 | 6.6% | 443.8 |
| Within-Run | S3 | NPK0062 | 7.8% | 475.1 |
| Within-Run | S3 | NPK0038 | 6.0% | 479.4 |
| Run to Run | S1 | NPK0016 | 0.0% | 36.8 |
| Run to Run | S1 | NPK0062 | 0.0% | 38.6 |
| Run to Run | S1 | NPK0038 | 0.0% | 37.1 |
| Run to Run | S2 | NPK0016 | 0.0% | 197.1 |
| Run to Run | S2 | NPK0062 | 0.0% | 209.3 |
| Run to Run | S2 | NPK0038 | 0.0% | 210.7 |
| Run to Run | S3 | NPK0016 | 0.0% | 443.8 |
| Run to Run | S3 | NPK0062 | 0.0% | 475.1 |
| Run to Run | S3 | NPK0038 | 0.0% | 479.4 |
| Day to Day | S1 | NPK0016 | 2.3% | 36.8 |
| Day to Day | S1 | NPK0062 | 2.2% | 38.6 |
| Day to Day | S1 | NPK0038 | 1.7% | 37.1 |
| Day to Day | S2 | NPK0016 | 1.4% | 197.1 |
| Day to Day | S2 | NPK0062 | 0.0% | 209.3 |
| Day to Day | S2 | NPK0038 | 2.1% | 210.7 |
| Day to Day | S3 | NPK0016 | 3.6% | 443.8 |
| Day to Day | S3 | NPK0062 | 0.0% | 475.1 |
| Day to Day | S3 | NPK0038 | 2.1% | 479.4 |
| Total Precision | S1 | NPK0016 | 7.0% | 36.8 |
| Total Precision | S1 | NPK0062 | 7.8% | 38.6 |
| Total Precision | S1 | NPK0038 | 7.9% | 37.1 |
| Total Precision | S2 | NPK0016 | 7.4% | 197.1 |
| Total Precision | S2 | NPK0062 | 7.0% | 209.3 |
| Total Precision | S2 | NPK0038 | 6.6% | 210.7 |
| Total Precision | S3 | NPK0016 | 7.5% | 443.8 |
| Total Precision | S3 | NPK0062 | 7.8% | 475.1 |
| Total Precision | S3 | NPK0038 | 6.4% | 479.4 |

### Example 3

The following data show the precision of the two biomarker assays (AM-1091 and AM-1384) on our test cartridge. These data show the precision (CV) of clinical sample results. Twenty-one (21) patient samples were tested on a single lot of test cartridges. At least 4 replicate measurements were conducted on each sample. For each sample, the results of the replicate measurements were averaged and also used to calculate the standard deviation and CV of the sample results. As shown in the results below (Table 16), both assays have about 10% CV's or less.

TABLE 16

| | | | AM-1384 | | | AM-1091 | |
|---|---|---|---|---|---|---|---|
| | | AM-1384 | Std. Dev. | | AM-1091 | Std. Dev. | |
| Clinical Sample | N | Mean Conc. | Conc. | CV | Mean Conc. | Conc. | CV |
| 10A | 4 | 78.86 | 3.02 | 3.8% | 2.96 | 0.10 | 3.5% |
| 111AAu0agiX0C | 4 | 164.28 | 8.54 | 5.2% | 5.14 | 0.21 | 4.2% |
| 111EHu0areX0J | 4 | 166.53 | 5.30 | 3.2% | 6.77 | 0.30 | 4.4% |
| 111KGu0aspX0H | 4 | 363.03 | 8.74 | 2.4% | 12.05 | 0.38 | 3.2% |
| 111MGu0aqhX06 | 4 | 77.95 | 2.67 | 3.4% | 2.57 | 0.20 | 7.7% |
| 11A | 4 | 79.12 | 1.44 | 1.8% | 2.16 | 0.19 | 8.6% |
| 12A | 4 | 91.72 | 3.54 | 3.9% | 3.77 | 0.17 | 4.6% |
| 13A | 4 | 62.01 | 3.50 | 5.6% | 2.04 | 0.14 | 6.7% |
| 15A | 4 | 236.01 | 3.54 | 1.5% | 9.52 | 0.53 | 5.5% |
| 17A | 4 | 111.77 | 6.36 | 5.7% | 14.10 | 0.78 | 5.5% |
| 21A | 4 | 79.21 | 2.08 | 2.6% | 3.75 | 0.14 | 3.8% |
| 22A | 4 | 75.39 | 3.42 | 4.5% | 1.77 | 0.19 | 10.6% |
| 23A | 4 | 64.49 | 2.36 | 3.7% | 3.89 | 0.12 | 3.0% |
| 24A | 4 | 129.11 | 4.05 | 3.1% | 4.98 | 0.15 | 3.1% |
| 26A | 4 | 66.36 | 2.42 | 3.6% | 3.54 | 0.37 | 10.5% |
| 28A | 4 | 101.27 | 5.95 | 5.9% | 3.09 | 0.15 | 4.9% |
| 29A | 4 | 100.34 | 3.12 | 3.1% | 3.70 | 0.08 | 2.1% |
| 30A | 4 | 177.62 | 4.74 | 2.7% | 4.99 | 0.17 | 3.3% |
| 7A | 4 | 200.54 | 9.55 | 4.8% | 6.58 | 0.24 | 3.7% |
| 8A | 7 | 80.23 | 5.89 | 7.3% | 4.05 | 0.14 | 3.5% |
| 9A | 4 | 84.05 | 2.99 | 3.6% | 4.46 | 0.16 | 3.6% |

1. (canceled)

2. A method for quantitatively detecting multiple analytes in a sample, which method comprises:
a) contacting a liquid sample with a test device that comprises a porous matrix that comprises at least two distinct test locations on said porous matrix, each of said test locations comprising a test reagent that binds to an analyte or another binding reagent that binds to said analyte, or is an analyte or an analyte analog that competes with an analyte in said sample for binding to a binding reagent for said analyte, and said test reagents at said at least two test locations bind to at least two different analytes or different binding reagents that bind to said different analytes, or are different analytes or analyte analogs, wherein the liquid sample is applied to a site of the test device upstream of the test locations;
b) transporting multiple analytes, if present in the liquid sample, and a labeled reagent to the test locations; and
c) assessing a detectable signal at the test locations to determine the amounts of the multiple analytes in the sample, wherein the amount of each of the analytes is determined,
wherein the amount of each of the analytes is determined with a CV ranging from about 0.1% to about 10%.

3. (canceled)

4. (canceled)

5. The method of claim 1, wherein the test reagents specifically bind to at least two different analytes.

6. The method of claim 5, wherein the test reagents are antibodies.

7. The method of claim 1, wherein the detectable signal is generated by a labeled reagent comprises a label and a moiety that specifically binds to an analyte in the sample.

8. The method of claim 7, wherein the label is a soluble label, e.g., a fluorescent label.

9. The method of claim 7, wherein a developing liquid is used to transport the analytes and/or the labeled reagent to the test locations.

10. The method of claim 1, which is used for quantitatively detecting multiple analytes that are diagnostic, prognostic, risk assessment, stratification and/or treatment monitoring markers.

11. The method of claim 10, wherein the analytes are markers for diseases or conditions selected from the group consisting of infectious diseases, parasitic diseases, neoplasms, diseases of the blood and blood-forming organs, disorders involving the immune mechanism, endocrine, nutritional and metabolic diseases, mental and behavioural disorders, diseases of the nervous system, diseases of the eye and adnexam, diseases of the ear and mastoid process, diseases of the circulatory system, diseases of the respiratory system, diseases of the digestive system, diseases of the skin and subcutaneous tissue, diseases of the musculoskeletal system and connective tissue, diseases of the genitourinary system, pregnancy, childbirth and the puerperium, conditions originating in the perinatal period, congenital malformations, deformations, chromosomal abnormalities, injury, poisoning, consequences of external causes, external causes of morbidity and mortality.

12. The method of claim 10, wherein the analytes are markers for acute coronary syndrome (ACS), abdominal pain, cerebrovascular injury, kidney injury, e.g., acute kidney injury or chronic kidney disease, or sepsis.

13. The method of claim 12, wherein the markers for kidney injury are selected from the group consisting of insulin-like growth factor-binding protein 7 (or IGFBP7 or FSTL2 or IBP-7 or IGF-binding protein 7 or IGFBP-7 or IGFBP-7v or IGFBPRP1 or IGFBP-rP1 or MAC25 or MAC-25 or MAC 25 or PGI2-stimulating factor or AGM), Metallopeptidase inhibitor 2 (or CSC-21K or Metalloproteinase inhibitor 2 or TIMP-2 or Tissue inhibitor of metalloproteinases 2 or TIMP2 or TIMP 2), Neutrophil elastase (or Bone marrow serine protease or ELA2 or Elastase-2 or HLE or HNE or Human leukocyte elastase or Medullasin or Neutrophil elastase or PMN-E or PMN elastase or SCN1 or ELANE or elastase neutrophil expressed or elastase 2 or neutrophil-derived elastase or granulocyte-derived elastase or polymorphonuclear elastase or leukocyte elastase), hyaluronic acid (or Hyaluronan or hyaluronate), NGAL, KIM-1, Cystatin C, serum creatinine, L-FABP, IL-18, pi-GST, alph-GST, and Clusterin.

14. The method of claim 12, wherein the analytes are IGFBP7 and TIMP-2.

15. The method of claim 1, wherein each of the analytes has a concentration ranging from about 1 pg/ml to about 1 μg/ml, e.g., about 1 pg/ml, 10 pg/ml, 100 pg/ml, 1 ng/ml, 2 ng/ml, 3 ng/ml, 3.5 ng/ml, 4 ng/ml, 5 ng/ml, 6 ng/ml, 7 ng/ml, 8 ng/ml, 9 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 950 ng/ml, or higher.

16. The method of claim 1, wherein the test device further comprises machine-readable information, e.g., a barcode.

17. The method of claim 1, wherein a fluorescent conjugate comprising a biological reagent and a fluorescent molecule is used to generate a detectable signal at the test locations, and the fluorescent conjugate and/or the test device further comprises a means for impeding phototoxic degradation of the biological reagent or nonspecific binding of the fluorescent conjugate to the test device or a non-analyte moiety.

18. The method of claim 1, wherein the detectable signal is detected by a reader, e.g., a fluorescent reader.

19. The method of claim 18, wherein the reader comprises a light source and a photodetector that are positioned at the same side or different sides of the test device.

20. The method of claim 18, wherein each of the test locations comprises a capture region characterized by a first dimension transverse to the lateral flow direction and a second dimension parallel to the lateral flow direction, and the reader comprises an illumination system operable to focus a beam of light onto an area of the test locations having at least one surface dimension at most equal to smallest of the first and second dimensions of the capture region.
